(19) United States
(12) Patent Application Publication
(10) Pub. No.: US 2026/0357308 A1
(43) Pub. Date: Nov. 12, 2026

(54) ENGINEERED MICROBIAL CONSORTIA FOR UPCYCLING POLYETHYLENE TEREPHTHALATE

(71) Applicant: The Board of Trustees of the University of Illinois, Urbana, IL (US)

(72) Inventors: Ting LU, Savoy, IL (US); Teng BAO, Champaign, IL (US); Yuanchao QIAN, Champaign, IL (US); Yongping XIN, Champaign, IL (US)

(73) Assignee: The Board of Trustees of the University of Illinois, Urbana, IL (US)

(21) Appl. No.: 19/493,875

(22) PCT Filed: Jun. 24, 2024
(86) PCT No.: PCT/US2024/035249

§ 371 Date: Dec. 16, 2025

Related U.S. Application Data
(60) Provisional application No. 63/509,709, filed on Jun. 22, 2023.

(51) Int. Cl. C12P 7/625; C08J 11/10; C12N 15/78; C12P 7/46

(52) U.S. Cl. CPC C12P 7/625; C08J 11/105; C12N 15/78; C12P 7/46

(57) ABSTRACT

Plastic pollution is rapidly increasing worldwide, causing adverse impacts on the environment, wildlife, and human health. One tempting solution to this crisis is upcycling plastics into products with engineered microorganisms; however, this remains challenging due to complexity in conversion. Here we present a synthetic microbial consortium that efficiently degrades polyethylene terephthalate hydrolysate and subsequently produces desired chemicals through division of labor.

### PRIORITY

This application claims the benefit of U.S. Ser. No. 63/509,709, filed on Jun. 22, 2023, which is incorporated by reference herein in its entirety.

### STATEMENT REGARDING FEDERALLY SPONSORED RESEARCH OR DEVELOPMENT

This invention was made with government support under HR0011-20-2-0033 awarded by the Department of Defense/DARPA. The United States Government has certain rights in the invention.

### BACKGROUND

Plastic pollution is rapidly increasing worldwide, causing adverse impacts on the environment, wildlife, and human health. One tempting solution to this crisis is upcycling plastics into products with engineered microorganisms; however, this remains challenging due to complexity in conversion.

Current strategies for plastics waste mitigation include mechanical, thermal, and chemical recycling. However, mechanical recycling is hampered by complicated sorting and inferior properties of recycled products; thermal recycling results in incomplete combustion and release of toxic compounds; and chemical recycling is hindered by strict catalyst and processing requirements.

Microbial-based biodegradation is an attractive alternative for polymer breakdown, with the potential to simplify processing procedures and integrate waste degradation with product generation. However, engineered monocultures face challenges owing to the complexity of polymer upcycling. Typically, plastic depolymerization results in heterogeneous and mixed chemicals. For example, complete polyethylene terephthalate (PET) hydrolysis gives rise to terephthalic acid (TPA) and ethylene glycol (EG). The physiochemical heterogeneity of these chemicals demands microbes to encode multiple degradation pathways, which lead to heavy metabolic burdens. Concurrently, the chemicals are often toxic, imposing additional stresses on microbes particularly when they are abundant or crude hydrolysate is used. Additionally, to upcycle plastics, strains must carry heterologous biosynthetic pathways and synthesize intermediates and products, which are associated with extra burdens and toxicities.

### SUMMARY

An aspect provides a recombinant Pseudomonas putida group bacterium (Bacterium A) comprising a deleted or non-functional ped operon and recombinant nucleic acid molecules encoding a large subunit of terephthalate 1,2-dioxygenase (tpaAa), small subunit of terephthalate 1,2-dioxygenase (tpaAb), ferredoxin reductase subunit (tpaB), dihydrodiol dehydrogenase (tpaC), and terephthalate transporter (tpaK). The nucleic acid molecules encoding tpaAa, tpaAb, tpaB, tpaC, and tpaK can be derived from Rhodococcus jostii. Another aspect provides a recombinant Pseudomonas putida group bacterium (Bacterium B) comprising a deleted or non-functional repressor gene of the glycolate dehydrogenase (Gcl) pathway (gclR) and a recombinant strong constitutive promoter operably linked to a glcDEF operon. The strong constitutive promoter can be Tac Promoter (P_{tac}). The bacterium can further comprise a recombinant ribosome binding site operably linked to the glcDEF operon. An aspect provides a bacterial culture comprising the Pseudomonas putida group Bacterium A and the Pseudomonas putida group Bacterium B.

Another aspect provides a method of degrading polyethylene terephthalate (PET) comprising mixing a hydrolyzed PET product with a growth medium, and adding the bacterial culture of Bacterium A and Bacterium B to a mixture of the hydrolyzed PET product and the growth medium. The hydrolyzed PET product can be formed by depolymerizing PET by hydrolysis. The hydrolysis can comprise contacting the PET with 0.5 to 5% NaOH. 50% or more of terephthalic acid (TPA) and ethylene glycol (EG) present in the hydrolyzed PET product is degraded.

Yet another aspect provides a recombinant Pseudomonas putida group bacterium (Bacterium C) comprising:
(i) a deleted or non-functional ped operon;
(ii) recombinant nucleic acid molecules encoding tpaAa, tpaAb, tpaB, tpaC, and tpaK; and
(iii) recombinant nucleic acid molecules encoding hydroxyacyl-ACP acyltransferase (phaG), acyl-CoA-synthase (alkK), mcl-PHA synthase 1 (phaC1) and mcl-PHA synthase 2 (phaC2).

Yet another aspect provides a recombinant Pseudomonas putida group bacterium (Bacterium D) comprising:
(i) a deleted or non-functional gclR gene;
(ii) a recombinant strong constitutive promoter operably linked to a glcDEF operon; and
(iii) recombinant nucleic acid molecules encoding phaG, alkK, phaC1 and phaC2.

An aspect provides a bacterial culture comprising the Pseudomonas putida group Bacterium C and the Pseudomonas putida group Bacterium D.

Another aspect provides a method of making medium chain length polyhydroxyalkanoates (mcl-PHA (C6 to C14)) comprising mixing a hydrolyzed PET product with a growth medium, and adding the bacterial culture Bacterium C and Bacterium D to a mixture of the hydrolyzed PET product and the growth medium, wherein mcl-PHA (C6-C14) is produced. The hydrolyzed PET product can be formed by depolymerizing PET by hydrolysis. The hydrolysis can comprise contacting the PET with 0.5 to 5% NaOH.

Yet another aspect provides a recombinant Pseudomonas putida group bacterium (Bacterium E) comprising:
(i) a deleted or non-functional ped operon;
(ii) recombinant nucleic acid molecules encoding tpaAa, tpaAb, tpaB, tpaC, and tpaK.
(iii) a deleted or non-functional catRBCA cluster;
(iv) a recombinant nucleic acid molecule encoding PCA decarboxylase (aro Y); and
(v) a recombinant nucleic acid molecule encoding decarboxylase enhancer (ecdB).

Another aspect provides recombinant Pseudomonas putida group bacterium (Bacterium F) comprising:
(i) a deleted or non-functional gclR gene;
(ii) a recombinant strong constitutive promoter operably linked to a glcDEF operon
(iii) a deleted or non-functional catRBCA cluster; and
(iv) a recombinant strong constitutive promoter operably linked to a nucleic acid molecule encoding major catechol 1,2-dioxygenase (catA).

An aspect provides a bacterial culture comprising the Pseudomonas putida group Bacterium E and the Pseudomonas putida group Bacterium F.

Another aspect provides method of making muconate (MA) comprising mixing a hydrolyzed PET product with a growth medium, and adding the recombinant Pseudomonas putida group Bacterium E and the recombinant Pseudomonas putida group Bacterium F to a mixture of the hydrolyzed PET product and the growth medium, wherein MA is produced. The hydrolyzed PET product is formed by depolymerizing PET by hydrolysis. The hydrolysis can comprise contacting the PET with 0.5 to 5% NaOH. The recombinant Pseudomonas putida group Bacterium F can be added to the mixture of the hydrolyzed product and the growth medium before the recombinant Pseudomonas putida group Bacterium E is added to the mixture.

Yet another aspect provides a recombinant P. putida group bacterium (Bacterium G) comprising a deleted or non-functional ped operon and recombinant nucleic acid molecules encoding a large subunit of terephthalate 1,2-dioxygenase (tpaAa), small subunit of terephthalate 1,2-dioxygenase (tpaAb), ferredoxin reductase subunit (tpaB), dihydrodiol dehydrogenase (tpaC), and terephthalate transporter (tpaK), reduced expression of icd, a recombinant nucleic acid molecule encoding acn, recombinant nucleic acid molecules encoding phaG, alkK, phaC1, and phaC2, which are operably linked to one or more strong constitutive promoters and a recombinant nucleic acid molecule encoding cadA. The one or more strong constitutive promoters can be P_{tac}. The reduced expression of icd can be due to a weak start codon or a recombinant weak promoter.

An aspect provides recombinant P. putida group bacterium (Bacterium H) comprising a deleted or non-functional repressor gene of the glycolate dehydrogenase (Glc) pathway (glcR), and a recombinant strong constitutive promoter operably linked to a glcDEF operon, a recombinant ribosome binding site operably linked to the glcDEF operon, reduced expression of icd, a recombinant nucleic acid molecule encoding acn, recombinant nucleic acid molecules encoding phaG, alkK, phaC1, and phaC2 operably linked to one or more strong or constitutive promoters, and a recombinant nucleic acid molecule encoding cadA. The reduced expression of icd can be due to a weak start codon or a recombinant weak promoter. The strong constitutive promoter can be P_{tac}.

Yet another aspect provides a bacterial culture comprising the Pseudomonas putida group Bacterium G and the Pseudomonas putida group Bacterium H.

Even another aspect provides a method of simultaneous mcl-PHA and itaconic acid production comprising mixing a hydrolyzed PET product with a growth medium, and adding the recombinant Pseudomonas putida group Bacterium G and the recombinant Pseudomonas putida group Bacterium H to a mixture of the hydrolyzed PET product and the growth medium, wherein mcl-PHA and itaconic acid is produced. The hydrolyzed PET product can be formed by depolymerizing PET by hydrolysis. The hydrolysis can comprise contacting the PET with 0.5 to 5% NaOH.

### BRIEF DESCRIPTION OF THE DRAWINGS

FIG. 1A-1J: A synthetic P. putida consortium for simultaneous TPA and EG degradation. 1A, Schematic of the designer T-E consortium composed of two strains Pp-T and Pp-E. Pp-T specializes in TPA degradation, which was developed by deleting the ped operon and constitutively expressing the genes TpaAa, TpaAb, TpaB, TpaC and TpaK. By contrast, Pp-E specializes in EG degradation, achieved by knocking out the gclR gene to alleviate the expression of the genes it regulates (Gcl, GlxR, Hyi, TtuD and PykA) and replacing the native promoter of the glcDEF operon with a strong constitutive promoter (P_{tac}). 1B-1D, Temporal profiles of substrate degradation and cell growth during TPA (1B), EG (1C), and TPA and EG mixture (1D) fermentations by Pp-T. 1E-1G, Temporal profiles of substrate degradation and cell growth during TPA (1E), EG (1F), and TPA and EG (1G) fermentations by Pp-E. 1H-J, Temporal profiles of the TPA (1H), EG (1I), TPA and EG (1J) fermentations by the T-E consortium. The co-culture was inoculated at a 1:1 ratio. For panels 1A, bold text indicates overexpressed genes whereas cross (‘x’) indicates the deletion of genes in italics. Experimental data are presented as mean values with standard deviations from three independent experiments.

FIG. 2A-F: Comparison of TPA and EG co-degradation by the T-E consortium and a single co-utilization strain. 2A, Schematic of a single strain (Pp-TE) capable of TPA and EG co-consumption. The entire tpa cluster was introduced and constitutively expressed in Pp-E to achieve TPA and EG co-degradation. 2B-C, Temporal profiles of substrate degradation and cell growth during TPA (2B) and EG (2C) fermentations by Pp-TE. 2D-2E, Fermentation profiles of the T-E consortium (2D) and Pp-E (2E) during a mixed TPA and EG fermentation. The consortium was inoculated at a 3:1 ratio for Pp-T and Pp-E. 2F, Comparison of the degradation times of the T-E consortium and the single strain Pp-TE for various TPA and EG mixtures. The color of each grid indicates the time needed to fully degrade the corresponding mixture, which is also directly shown on the grid. For panels 2A, bold text indicates overexpressed genes whereas cross (‘x’) indicates the deletion of genes in italics. Experimental data are presented as mean values with standard deviations from three independent experiments.

FIG. 3A-3G: Integrated chemical and microbial processing for PET deconstruction. 3A, Schematic illustration of an integrated PET processing workflow. PET is depolymerized through alkaline hydrolysis to generate a solution containing TPA and EG. The solution is then added with M9 medium to form a growth medium. Subsequently, the medium is inoculated with engineered microbes to fulfill fermentations to realize a full degradation. 3B, The yield (%) of Na₂TPA and EG from PET powder under various hydrolysis conditions. 3C, Characterization of the crude hydrolysis product using pure TPA and EG as standard samples, confirming successful TPA and EG release from PET. 3D-3G, Temporal profiles of a direct PET hydrolysate fermentation by the Pp-T (3D), Pp-E (3E), T-E consortium (3F) and the single strain Pp-TE (3G). The Pp-T and Pp-E co-culture was inoculated at a 3:1 ratio. Experimental data are presented as mean values with standard deviations from three independent experiments.

FIG. 4A-4K: Upcycling of hydrolyzed PET into mcl-PHA. 4A, Design of a synthetic consortium that produces mcl-PHA from TPA and EG co-degradation. The consortium was created by introducing an mcl-PHA biosynthetic pathway into Pp-T and Pp-E along with additional knockouts. 4B, Design of an engineered strain that synthesizes mcl-PHA and co-utilizes TPA and EG. The strain was developed by adding an mcl-PHA synthesis pathway into Pp-TE along with additional knockouts. 4C-4E, Comparison of mcl-PHA production by different Pp-T (4C), Pp-E (4D) and Pp-TE (4E) derivatives. Pp-T has three derivates: Pp-TS, Pp-TP and PpA-TP. Pp-E has three derivates: Pp-ES, Pp-EP and PpA-EP. Pp-TE has three derivates: Pp-TES, Pp-TEP and PpA-TEP. 4F-4G Temporal profiles of direct PET hydrolysate fermentations by the TP-EP consortium (4F) and Pp-TEP (4G). An initial 1:3 inoculation ratio was used for Pp-TP and Pp-EP. 4H-4 Temporal profiles of fed-batch fermentations by the TP-EP consortium (4H) and Pp-TEP (4I). An initial 1:3 inoculation ratio was used for Pp-TP and Pp-EP. 4J, The mcl-PHA titer and monomer composition of mcl-PHA produced by the TP-EP consortium and single-strain counterparts Pp-TEP in batch and fed-batch cultures. 4K, The mcl-PHA yield from dry cell of TP-EP consortium and single-strain counterparts Pp-TEP in batch and fed-batch cultures. For panels 4A and 4B, bold text indicates overexpressed genes whereas cross (‘x’) indicates the deletion of genes in italics. In panels 4C, 4D, 4E, 4J and 4K, mcl-productions were calculated at the time point when TPA and/or EG were fully degraded. In panels 4D, 4D, 4E and 4K results were compared using ANOVA test (****, p<0.0001; ***, p<0.0002; **, p<0.0021; *, p<0.0332; ns, not significant). Experimental data are presented as mean values with standard deviations from three independent experiments.

FIG. 5A-5K: MA production via rational pathway engineering and DOL-mediated optimization. 5A, Design of a synthetic consortium that upcycles PET hydrolysate into MA. The consortium (TC-EM) consists of Pp-TC and Pp-EM derived from Pp-T and Pp-E, respectively. Involving two layers of DOL, the strains were divided to specialize in TPA (Pp-TC) and EG (Pp-EM) consumptions and to enable the TPA-to-CAT (Pp-TC) and CAT-to-MA (Pp-EM) conversions. 5B, Temporal profiles of TPA fermentation by Pp-TC that yields CAT. 5C, Temporal profiles of EG fermentation by Pp-EM that upgrades CAT into MA. 5D, Design of a single strain (Pp-TEM) that produces MA from PET hydrolysate. 5E, Temporal profiles of mixed TPA and EG fermentation by Pp-TEM that converts TPA and EG into MA. 5F, Production optimization by modulating the initial strain inoculation ratio of the TC-EM consortium. 5G, Temporal profiles of mixed TPA and EG fermentation by the TC-EM consortium with a 1:5 initial ratio. 5H, Comparison of the final MA production by the TC-EM consortium with different initial ratios and Pp-TEM. 5I, Colors of the TC-EM co-culture and Pp-TEM monoculture through the course of hydrolysate fermentation. 5J, Production optimization by altering the lag time between Pp-TC and Pp-EM inoculations. 5K, Comparison of final products and remnant substrates of the hydrolysate fermentation by the TC-EM consortium with varied inoculation lag times. For panels 5A and 5D, bold text indicates overexpressed genes whereas cross (‘x’) indicates the deletion of genes in italics. Experimental data are presented as mean values with standard deviations from three independent experiments.

FIG. 6A-6E: mcl-PHA and IA co-production from PET hydrolysate. 6A, Design of a synthetic consortium (TAPI-EAPI) that simultaneously produces mcl-PHA and IA through TPA and EG co-degradation. The consortium involves two strains, Pp-TAPI and Pp-EAPI, which assimilate TPA and EG, respectively, and convert them to mcl-PHA and IA. Here, bold text indicates overexpressed genes whereas cross (‘x’) indicates the weakening of the expression of genes in italics. 6B-6C, Comparison of mcl-PHA and IA production by different Pp-T (6B) and Pp-E (6C) derivatives using pure TPA and EG mix as the carbon substrates. Relevant genetic modifications of the derivatives include the presence (+) or absence (−) of modifications improving cis-aconitate, mcl-PHA, and IA flux. Pp-T has three derivatives: Pp-TA, Pp-TAP and Pp-TAPI. Pp-E also have three derivatives, including Pp-EA, Pp-EAP and Pp-EAPI. 6D-6E, Temporal profiles of TPA, EG and OD (6D) and corresponding mcl-PHA and IA titers (6E) during a PET hydrolysate fermentation by the TAPI-EAPI consortium. Experimental data are presented as mean values with standard deviations from three independent experiments.

FIG. 7A-7B: TPA degradation by the Pp-T strain under various EG levels. 7A, Temporal profiles of TPA degradation (left) and cell growth (right) of Pp-T in the presence of TPA only. Initial TPA concentrations tested include 10, 31.6, 100, 178 and 316 mM. 7B, Temporal profiles of TPA degradation (top left), cell growth (top right), EG degradation (bottom left) and glycolate accumulation (bottom right) of Pp-T under 56.2 mM TPA mixed with varied EG levels (0, 31.6, 56.2, 100 and 178 mM). Experimental data are presented as mean values with standard deviations from three independent experiments.

FIG. 8A-8B: Comparison of metabolite concentrations and cell growth TPA-utilizing strains Pp-T₀ and Pp-T. 8A-8B, Temporal profiles of TPA degradation (top left), cell growth (top right), EG degradation (middle left), glycolate accumulation (middle right), glyoxylate accumulation (bottom left), and succinate accumulation (bottom right) of Pp-T₀ (8A) and Pp-T (8B). In both panels, a fixed 100 mM of TPA (100 mM) but varied EG concentrations (10, 31.6, 100, 178 and 316 mM) were used as different initial conditions. Experimental data are presented as mean values with standard deviations from three independent experiments.

FIG. 9A-9D: EG degradation by Pp-E and its parental strains under various TPA levels. 9A, Temporal profiles of cell growth (top left), growth rate (top right), EG degradation (middle left), EG consumed rate (middle right), glycolate accumulation (bottom left) and glyoxylate (bottom right) of P. putida EM42, M31, PpM01 and Pp-E (PpM02) in the presence of 56 mM EG only. 9B, Temporal profiles of EG degradation (left) and cell growth (left) of Pp-E in the presence of EG only. Initial EG concentrations tested include 10, 31.6, 100, 178 and 316 mM. 9C-9D, Temporal profiles of EG degradation (left) and cell growth (right) of Pp-E for different initial TPA and EG mixtures. Panel 9C corresponds to 56.2 mM of EG and varied initial TPA concentrations (0, 31.6, 56.2, 100 and 178 mM). Panel 9D corresponds to 100 mM of initial EG level (100 mM) but varied initial TPA concentrations (10, 31.6, 100, 178 and 316 mM). In panel 9A, the results of growth rate and EG consumed rate were compared using ANOVA test (****, p<0.0001; ***, p<0.0002). Experimental data are presented as mean values with standard deviations from three independent experiments.

FIG. 10A-10B: TPA and EG degradation by the Pp-TE strain. 10A, Temporal profiles of TPA degradation (left) and cell growth (right) of Pp-TE in the presence of TPA only. Initial TPA concentrations tested include 10, 31.6, 100, 178 and 316 mM. 10B, Temporal profiles of the EG degradation (left) and cell growth (right) of Pp-TE in the presence of EG only. Initial EG concentrations tested include 10, 31.6, 100, 178 and 316 mM. Experimental data are presented as mean values with standard deviations from three independent experiments.

FIG. 11: Temporal profiles of substrate degradation and cell growth of the T-E consortium and Pp-TE under various TPA and EG mixtures. A total of 32 fermentations were carried out to systematically compare the substrate degradation rates of the T-E consortium and Pp-TE under 16 substrate conditions. A 3:1 inoculation ratio was used for Pp-T and Pp-E. Experimental data are presented as mean values with standard deviations from three independent experiments.

FIG. 12A-12D: Fermentation of PET hydrolysate by various strains. a,b, Temporal fermentation profiles of the T-E consortium (12A) and Pp-TE (12B) with 31.6 mM of initial PET hydrolysate. c,d, Temporal fermentation profiles of the T-E consortium (12C) and Pp-TE (12D) with 100 mM of initial PET hydrolysate. The measured TPA and EG concentrations deviate slightly from 100 mM due to the minor TPA precipitation at a high concentration. Experimental data are presented as mean values with standard deviations from three independent experiments.

FIG. 13A-13I: TPA and EG degradation by various PHA production strains. 13A-13C, Substrate degradation and cell growth during TPA fermentation by the strains Pp-TS (13A), Pp-TP (13B) and PpΔ-TP (13C), respectively. The initial TPA concentration was 56 mM. 13D-13F, Substrate degradation and cell growth during EG fermentation by the strains Pp-ES (13D), Pp-EP (13E) and PpΔ-EP (13F), respectively. The initial EG concentration was 56 mM. 13G-13I, Substrate degradations and cell growth during mixed TPA and EG fermentation by the strains Pp-TES (13G), Pp-TEP (13H) and PpΔ-TEP (i), respectively. The initial concentrations of TPA and EG were both 56 mM. Experimental data are presented as mean values with standard deviations from three independent experiments.

FIG. 14: Images of M9 medium containing CAT over time.

FIG. 15A-15G: CAT-to-MA conversion by Pp-EM using EG as the substrate. 15A-15D, Temporal fermentation and CAT-to-MA conversion profiles of Pp-EM for 56 mM EG mixed with 0 mM (15A), 2.5 mM (15B), 5.0 mM (15C) and 7.5 mM (15D) of CAT. 15E-15F, Temporal fermentation and CAT-to-MA conversion profiles of Pp-ES with 56 mM EG and 0 mM (15E) or 2.5 mM (15F) of CAT. 15G, Color change of the Pp-EM or Pp-ES culture throughout the fermentations in panels 15A-15F. In panels 15B-15D, the measured OD₆₀₀ was not accurate due to the precipitation of “brown carbon”. Experimental data are presented as mean values with standard deviations from three independent experiments.

FIG. 16A-16E: MA production by Pp-EM with CAT added after 12 h of fermentation. The fermentations started with 56 mM EG at 0 h but were supplemented at 12 h with various levels of CAT, including 2.5 mM (16A), 5.0 mM (16B), 7.5 mM (16C), 10.0 mM (16D) and 12.5 mM (16E). The temporal profiles of EG, CAT, MA and OD₆₀₀ during the fermentations are shown. Experimental data are presented as mean values with standard deviations from three independent experiments.

FIG. 17A-17F: MA production from PET hydrolysate by the TC-EM consortium by altering the initial population ratio. 17A-17D, Temporal profiles of TPA, EG, CAT and MA during the PET hydrolysate fermentation by the TC-EM consortium with different initial Pp-TC: Pp-EM ratios including 3:1 (17A), 1:1 (17B), 1:3 (17C), and 1:9 (17D). 17E, Temporal profiles of TPA, EG, CAT and MT during the PET hydrolysate fermentation by the single strain Pp-TEM. 17F, Images of the fermentation broths at 96 h for the experiments in panels 17A-17E and FIG. 5G. Experimental data are presented as mean values with standard deviations from three independent experiments.

FIG. 18A-18F: MA production from PET hydrolysate by the TC-EM consortium by varying the inoculation lag time. 18A-18D, Temporal profiles of TPA, EG, CAT and MA during the PET hydrolysate fermentation by the TC-EM consortium with different lag times between Pp-TC and Pp-EM inoculations, including 0 h (18A), 12 h (18B), 24 h (18C) and 36 h (18D). 18E, Color change of the TC-EM consortium culture throughout the fermentations in panels 18A-18D. 18F, Images of the final fermentation broths at 96 h for the experiments in panels 18A-18D. Experimental data are presented as mean values with standard deviations from three independent experiments.

FIG. 19A-19F: TPA and EG degradation by various mcl-PHA or/and IA production strains. 19A-19C, Substrate degradation and cell growth during pure TPA fermentation by the strains Pp-TA (19A), Pp-TAP (19B) and Pp-TAPI (19C), respectively. 19D-19F, Substrate degradation and cell growth during pure EG fermentation by the strains Pp-EA (19D), Pp-EAP (19E) and Pp-EAPI (19F), respectively. The initial concentrations of TPA and EG were both 56 mM. Experimental data are presented as mean values with standard deviations from three independent experiments.

### DETAILED DESCRIPTION

Aspects of the present disclosure include systems and methods for plastic waste degradation and upcycling. Compositions and methods described herein can be used in the sustainable production of high valued products from plastic waste.

Aspects of the present disclosure include a synthetic microbial consortium that efficiently degrades polyethylene terephthalate hydrolysate and subsequently produces desired chemicals through division of labor. Engineering microbial Division of Labor allows one to leverage the specialty of individual members to enhance overall system performance and expand design space. It also reduces the burdens of de novo pathways and alleviates the toxicities of substrates, intermediates, and products, thereby allowing engineered strains to maintain a metabolic homeostasis for function implementation. In certain aspects, the consortium involves two Pseudomonas putida group strains, specializing in terephthalic acid and ethylene glycol utilization respectively, to achieve complete degradation. Compared with its monoculture counterpart, the consortium exhibits faster deconstruction, particularly when substrate concentrations are high or crude hydrolysate is used. In an aspect the TPA and/or EG are present in a substrate at about 60, 70, 75, 80, 90, 100, 200, 300, 400 mM or more e.g., about 75 mM or more). In an aspect the TPA and/or EG are present in a crude PET hydrolysate or PET hydrolysate at about 50, 60, 70, 75, 80, 90, 100, 200, 300, 400 mM or more e.g., about 50 or 100 mM or more). The consortium methods also outperform monoculture for upcycling when polyhydroxyalkanoates serves as a target product, and confers a flexible tunability to alleviate intermediate toxicity for muconate synthesis via pathway partitioning. This work demonstrates engineered microbial consortia as an effective upcycling platform, promising a path toward polymer circularity and environmental sustainability.

Plastics are foundational materials that support our modern society. However, their remarkable versatility and limited recyclability have caused a growing problem of plastic pollution, adversely impacting the environment, wildlife, and human health worldwide. Recently, microbial-based biodegradation is emerging as an attractive alternative to mechanical, thermal, and chemical strategies for polymer breakdown, with the potential to simplify processing procedures and integrate waste degradation with product generation.

Despite these exciting developments, current biodegradation that focuses on engineered monocultures faces challenges owing to the complexity of polymer upcycling. First, plastic depolymerization typically yields mixed chemicals which can induce catabolite crosstalk during microbial degradation. For example, complete PET hydrolysis produces a mixture of terephthalic acid (TPA) and ethylene glycol (EG) that are heterogeneous in their physiochemical characteristics. In the presence of such mixed substrates, carbon catabolite repression between metabolic pathways is a ubiquitous nature of microorganisms that makes simultaneous multi-substrate fermentation a grand challenge in microbial metabolic engineering. Second, chemicals derived from depolymerization are often toxic and can impose various stresses on microbes, particularly when they are abundant or crude hydrolysate is used. Third, to upgrade plastic waste into valued products, strains must encode designer biosynthetic pathways and make intermediates and final products, which often bring additional burdens and toxicities and, hence, demand system-level fine-tuning for optimal biosynthesis and growth. Thus, plastic upcycling calls for microbial engineering with explicit consideration of crosstalk, burden, and toxicity as well as the flexibility for systematic optimization.

Consortium-based microbial engineering is a versatile strategy that is potentially superior to single-strain construction. Lying at the heart of microbial consortia is the division of labor (DOL), a form of organization through which a goal is divided into subtasks among constituting members. In principle, DOL allows each strain to specialize in one designated task without compromising its specialty for fulfilling multiple purposes. It also promises to reduce carbon catabolite crosstalk ubiquitous in the presence of mixed substrates by partitioning degradation pathways among members. Additionally, DOL may reduce the metabolic burdens and toxicities of substrates, intermediates, and products of individual strains, thereby helping cells to maintain metabolic homeostasis for functionalization. Moreover, by compartmentalizing subsystems, DOL promotes the orthogonality of modules and fosters systematic pathway fine-tuning and optimization.

Here, microbial consortia can be applied for plastic upcycling, a complex process involving mixed chemicals, inducing potential catabolic crosstalk and toxicity, and requiring systematic optimization. A synthetic microbial consortium comprising two Pseudomonas putida group strains was designed, which individually specialize in TPA and EG utilization, respectively, but collectively achieve complete degradation of PET hydrolysate. For comparison, a single strain was designed for TPA and EG co-consumption. The efficiency of this pair of engineered systems was compared under various substrate conditions, extended to upcycle PET by adopting medium chain length polyhydroxyalkanoates (mcl-PHA) and muconate (MA) as value-added products, and further demonstrated the feasibility of simultaneous polyhydroxyalkanoates and itaconic acid co-production. Together, these results showcase the potential of engineered consortia for plastic degradation and conversion, shedding light on the biological mitigation of growing plastic waste.

### Polynucleotides and Genes

Polynucleotides contain less than an entire microbial genome and can be single- or double-stranded nucleic acids. A polynucleotide can be RNA, DNA, cDNA, genomic DNA, chemically synthesized RNA or DNA or combinations thereof. A polynucleotide can comprise, for example, a gene, open reading frame, non-coding region, or regulatory element.

A gene is any polynucleotide molecule that encodes a polypeptide, protein, or fragment thereof, optionally including one or more regulatory elements preceding (5′ non-coding sequences) and following (3′ non-coding sequences) the coding sequence. In one aspect, a gene does not include regulatory elements preceding and following the coding sequence. A native or wild-type gene refers to a gene as found in nature, optionally with its own regulatory elements preceding and following the coding sequence. A chimeric or recombinant gene refers to any gene that is not a native or wild-type gene, optionally comprising regulatory elements preceding and following the coding sequence, wherein the coding sequences and/or the regulatory elements, in whole or in part, are not found together in nature. Thus, a chimeric gene or recombinant gene can comprise regulatory elements and coding sequences that are derived from different sources, or regulatory elements and coding sequences that are derived from the same source, but arranged differently than is found in nature. A gene can encompass full-length gene sequences (e.g., as found in nature and/or a gene sequence encoding a full-length polypeptide or protein) and can also encompass partial gene sequences (e.g., a fragment of the gene sequence found in nature and/or a gene sequence encoding a protein or fragment of a polypeptide or protein). A gene can include modified gene sequences (e.g., modified as compared to the sequence found in nature). Thus, a gene is not limited to the natural or full-length gene sequence found in nature.

Polynucleotides can be purified free of other components, such as proteins, lipids and other polynucleotides. For example, the polynucleotide can be 50%, 75%, 90%, 95%, 96%, 97%, 98%, 99%, or 100% purified. A polynucleotide existing among hundreds to millions of other polynucleotide molecules within, for example, cDNA or genomic libraries, or gel slices containing a genomic DNA restriction digest are not to be considered a purified polynucleotide. Polynucleotides can encode the polypeptides described herein (e.g., a pedD polypeptide, a pedE polypeptide, a pedF polypeptide, a pedG polypeptide, a pedH polypeptide, a pedI polypeptide, a glcR polypeptide, a catR polypeptide, a catB polypeptide, a catC polypeptide, a catA polypeptide, a tpaAa polypeptide, a tpaAb polypeptide, a tpaB polypeptide, a tpaC polypeptide, a tpaK polypeptide, a glcD polypeptide, a glcE polypeptide, a glcF polypeptide, a phaG polypeptide, an alkK polypeptide, a phaC1 polypeptide, a phaC2 polypeptide, an aroY polypeptide, and/or an ecdB polypeptide) and mutants or variants thereof.

Polynucleotides can comprise other nucleotide sequences, such as sequences coding for linkers, signal sequences, TMR stop transfer sequences, transmembrane domains, or ligands useful in protein purification such as glutathione-S-transferase, histidine tag, and Staphylococcal protein A.

Polynucleotides can be codon optimized for expression in bacteria, such as Pseudomonas, e.g., Pseudomonas putida group strains.

Polynucleotides can be isolated. An isolated polynucleotide is a naturally-occurring polynucleotide that is not immediately contiguous with one or both of the 5′ and 3′ flanking genomic sequences that it is naturally associated with. An isolated polynucleotide can be, for example, a recombinant DNA molecule of any length, provided that the nucleic acid sequences naturally found immediately flanking the recombinant DNA molecule in a naturally-occurring genome is removed or absent. Isolated polynucleotides also include non-naturally occurring nucleic acid molecules. Polynucleotides can encode full-length polypeptides, polypeptide fragments, and variant or fusion polypeptides.

Degenerate polynucleotide sequences encoding polypeptides described herein, as well as homologous nucleotide sequences that are at least about 80, or about 85, 90, 91, 92, 93, 94, 95, 96, 97, 98, or 99% identical to polynucleotides described herein and the complements thereof are also polynucleotides. Degenerate nucleotide sequences are polynucleotides that encode a polypeptide described herein or fragments thereof, but differ in nucleic acid sequence from the wild-type polynucleotide sequence, due to the degeneracy of the genetic code. Complementary DNA (cDNA) molecules, species homologs, and variants of polynucleotides that encode biologically functional polypeptides also are polynucleotides.

Polynucleotides can be obtained from nucleic acid sequences present in, for example, a microorganism such as a yeast or bacterium. Polynucleotides can also be synthesized in the laboratory, for example, using an automatic synthesizer. An amplification method such as PCR can be used to amplify polynucleotides from either genomic DNA or cDNA encoding the polypeptides.

Polynucleotides can comprise coding sequences for naturally occurring polypeptides or can encode altered sequences that do not occur in nature.

Unless otherwise indicated, the term polynucleotide or gene includes reference to the specified sequence as well as the complementary sequence thereof.

The expression products of genes or polynucleotides are often proteins, or polypeptides, but in non-protein coding genes such as rRNA genes or tRNA genes, the product is a functional RNA. The process of gene expression is used by all known life forms, i.e., eukaryotes (including multicellular organisms), prokaryotes (bacteria and archaea), and viruses, to generate the macromolecular machinery for life. Several steps in the gene expression process can be modulated, including the transcription, up-regulation, RNA splicing, translation, and post-translational modification of a protein. Any process that deletes, reduces, or attenuates the expression of e.g., a pedD polypeptide, a pedE polypeptide, a pedF polypeptide, a pedG polypeptide, a pedH polypeptide, a pedI polypeptide, a glcR polypeptide, a catR polypeptide, a catB polypeptide, a catC polypeptide, or a catA polypeptide can be used to make a microorganism described herein.

### Polypeptides

A polypeptide is a polymer of two or more amino acids covalently linked by amide bonds. A polypeptide can be post-translationally modified. A purified polypeptide is a polypeptide preparation that is substantially free of cellular material, other types of polypeptides, chemical precursors, chemicals used in synthesis of the polypeptide, or combinations thereof. A polypeptide preparation that is substantially free of cellular material, culture medium, chemical precursors, chemicals used in synthesis of the polypeptide, etc., has less than about 30%, 20%, 10%, 5%, 1% or more of other polypeptides, culture medium, chemical precursors, and/or other chemicals used in synthesis. Therefore, a purified polypeptide is about 70%, 80%, 90%, 95%, 99% or more pure. A purified polypeptide does not include unpurified or semi-purified cell extracts or mixtures of polypeptides that are less than 70% pure.

The term “polypeptides” can refer to one or more of one type of polypeptide (a set of polypeptides). “Polypeptides” can also refer to mixtures of two or more different types of polypeptides (a mixture of polypeptides). The terms “polypeptides” or “polypeptide” can each also mean “one or more polypeptides.”

As used herein, the term “polypeptide of interest” or “polypeptides of interest”, “protein of interest”, “proteins of interest” includes any or a plurality of any of e.g., a pedD polypeptide, a pedE polypeptide, a pedF polypeptide, a pedG polypeptide, a pedH polypeptide, a pedI polypeptide, a glcR polypeptide, a catR polypeptide, a catB polypeptide, a catC polypeptide, a catA polypeptide, a tpaAa polypeptide, a tpaAb polypeptide, a tpaB polypeptide, a tpaC polypeptide, a tpaK polypeptide, a glcD polypeptide, a glcE polypeptide, a glcF polypeptide, a phaG polypeptide, an alkK polypeptide, a phaC1 polypeptide, a phaC2 polypeptide, an aroY polypeptide, and/or an ecdB polypeptide or other polypeptides described herein.

A mutated protein or polypeptide comprises at least one deleted, inserted, and/or substituted amino acid, which can be accomplished via mutagenesis of polynucleotides encoding these amino acids. Mutagenesis includes well-known methods in the art, and includes, for example, site-directed mutagenesis by means of PCR or via oligonucleotide-mediated mutagenesis as described in Sambrook et al., Molecular Cloning—A Laboratory Manual, 2nd ed., Vol. 1-3 (1989).

As used herein, the term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that comprise a common structural domain that is at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%, identical are defined herein as sufficiently similar will be sufficiently similar to the amino acid sequence of the polypeptides described herein. Such variants generally retain the functional activity of the polypeptides described herein. Variants include peptides that differ in amino acid sequence from the native and wild-type peptide, respectively, by way of one or more amino acid deletion(s), addition(s), and/or substitution(s). These may be naturally occurring variants as well as artificially designed ones.

The terms “sequence identity” or “percent identity” are used interchangeably herein. To determine the percent identity of two polypeptide molecules or two polynucleotide sequences, the sequences are aligned for optimal comparison purposes (e.g., gaps can be introduced in the sequence of a first polypeptide or polynucleotide for optimal alignment with a second polypeptide or polynucleotide sequence). The amino acids or nucleotides at corresponding amino acid or nucleotide positions are then compared. When a position in the first sequence is occupied by the same amino acid or nucleotide as the corresponding position in the second sequence, then the molecules are identical at that position. The percent identity between the two sequences is a function of the number of identical positions shared by the sequences (i.e., % identity=number of identical positions/total number of positions (i.e., overlapping positions)×100). In some aspects the length of a reference sequence aligned for comparison purposes is at least 80% of the length of the comparison sequence, and in some aspects is at least 90% or 100%. In an aspect, the two sequences are the same length.

Ranges of desired degrees of sequence identity are approximately 80% to 100% and integer values in between. Percent identities between a disclosed sequence and a claimed sequence can be at least 80%, at least 83%, at least 85%, at least 90%, at least 95%, at least 98%, at least 99%, at least 99.5%, or at least 99.9%. In general, an exact match indicates 100% identity over the length of the reference sequence.

Polypeptides and polynucleotides that are sufficiently similar to polypeptides and polynucleotides described herein (e.g., pedD, pedE, pedF, pedG, pedH, pedI, glcR, catR, catB, catC, catA, tpaAa, tpaAb, tpaB, tpaC, tpaK, glcD, glcE, glcF, phaG, alkK, phaC1, phaC2, aroY, and/or an ecdB polynucleotides or polypeptides) can be used herein. Polypeptides and polynucleotides that are about 70, 80, 85, 90, 91, 92, 93, 94 95, 96, 97, 98, 99 99.5% or more identical to polypeptides and polynucleotides described herein can also be used herein.

Polypeptides and polynucleotides that are sufficiently similar to polypeptides and polynucleotides described herein (e.g., pedD, pedE, pedF, pedG, pedH, pedI, glcR, catR, catB, catC, catA, tpaAa, tpaAb, tpaB, tpaC, tpaK, glcD, glcE, glcF, phaG, alkK, phaC1, phaC2, aroY, and/or an ecdB polynucleotides or polypeptides) can be used herein. Polypeptides and polynucleotides that about 70, 80, 85, 90, 95, 96, 97, 98, 99% or more identity to polypeptides and polynucleotides described herein (e.g., pedD, pedE, pedF, pedG, pedH, pedI, glcR, catR, catB, catC, catA, tpaAa, tpaAb, tpaB, tpaC, tpaK, glcD, glcE, glcF, phaG, alkK, phaC1, phaC2, aroY, and/or an ecdB polynucleotides or polypeptides) can also be used herein.

### Constructs and Cassettes

A recombinant construct is a polynucleotide having heterologous polynucleotide elements. Recombinant constructs include expression cassettes or expression constructs, which refer to an assembly that is capable of directing the expression of a polynucleotide or gene of interest. An expression cassette generally includes regulatory elements such as a promoter that is operably linked to (so as to direct transcription of) a polynucleotide and often includes a polyadenylation sequence as well.

An “expression cassette” refers to a fragment of DNA comprising a coding sequence of a selected gene (e.g., pedD, pedE, pedF, pedG, pedH, pedI, glcR, catR, catB, catC, catA, tpaAa, tpaAb, tpaB, tpaC, tpaK, glcD, glcE, glcF, phaG, alkK, phaC1, phaC2, aroY, and/or an ecdB polynucleotides) and, optionally, regulatory elements preceding (5′ non-coding sequences) and following (3′ non-coding sequences) the coding sequence can be required for expression of the selected gene product. Thus, an expression cassette is typically composed of: 1) a promoter sequence; 2) a coding sequence [“ORF”]; and optionally 3) a 3′ untranslated region (i.e., a terminator). The expression cassette is usually included within a vector, to facilitate cloning and transformation. Different expression cassettes can be transformed into different organisms including bacteria, yeast, plants, and mammalian cells, as long as the correct regulatory elements are used for each host.

A recombinant construct or expression cassette can be contained within a vector. In addition to the components of the recombinant construct, the vector can include, one or more selectable markers, a signal which allows the vector to exist as single-stranded DNA (e.g., a M13 origin of replication), at least one multiple cloning site, and an origin of replication (e.g., a SV40 or adenovirus origin of replication).

Generally, a polynucleotide or gene that is introduced into a genetically engineered organism is part of a recombinant construct. A polynucleotide can comprise a gene of interest, e.g., a coding sequence for a protein, or can be a sequence that is capable of regulating expression of a gene, such as a regulatory element like a promoter, an antisense sequence, a sense suppression sequence, or a miRNA sequence. A recombinant construct can include, for example, regulatory elements operably linked 5′ or 3′ to a polynucleotide encoding one or more polypeptides of interest. For example, a promoter can be operably linked with a polynucleotide encoding one or more polypeptides of interest when it is capable of affecting the expression of the polynucleotide (i.e., the polynucleotide is under the transcriptional control of the promoter). Polynucleotides can be operably linked to regulatory elements in sense or antisense orientation. The expression cassettes or recombinant constructs can additionally contain a 5′ leader polynucleotide. A leader polynucleotide can contain a promoter as well as an upstream region of a gene. The regulatory elements (i.e., promoters, enhancers, transcriptional regulatory regions, translational regulatory regions, and translational termination regions) and/or the polynucleotide encoding a signal anchor can be native/analogous to the host cell or to each other. Alternatively, the regulatory elements can be heterologous to the host cell or to each other. See, U.S. Pat. No. 7,205,453 and U.S. Patent Application Publication Nos. 2006/0218670 and 2006/0248616. The expression cassette or recombinant construct can additionally contain one or more selectable marker genes.

Methods for preparing polynucleotides operably linked to regulatory elements and expressing polypeptides in a host cell are well-known in the art. See, e.g., U.S. Pat. No. 4,366,246. A polynucleotide can be operably linked when it is positioned adjacent to or close to one or more regulatory elements, which direct transcription and/or translation of the polynucleotide.

A promoter is a nucleotide sequence that is capable of controlling the expression of a coding sequence or gene. Promoters are generally located 5′ of the sequence that they regulate. Promoters may be derived in their entirety from a native gene, can be composed of different elements derived from promoters found in nature, and/or comprise synthetic nucleotide segments. Those skilled in the art will readily ascertain that different promoters may regulate expression of a coding sequence or gene in response to a particular stimulus, e.g., in a cell- or tissue-specific manner, in response to different environmental or physiological conditions, or in response to specific compounds. Promoters are typically classified into two classes: inducible and constitutive. A constitutive promoter refers to a promoter that allows for continual transcription of the coding sequence or gene under its control.

An inducible promoter refers to a promoter that initiates increased levels of transcription of the coding sequence or gene under its control in response to a stimulus or an exogenous environmental condition. If inducible, there are inducer polynucleotides present therein that mediate regulation of expression so that the associated polynucleotide is transcribed only when an inducer molecule is present. A directly inducible promoter refers to a regulatory region, wherein the regulatory region is operably linked to a gene encoding a protein or polypeptide, where, in the presence of an inducer of the regulatory region, the protein or polypeptide is expressed. An indirectly inducible promoter refers to a regulatory system comprising two or more regulatory regions, for example, a first regulatory region that is operably linked to a first gene encoding a first protein, polypeptide, or factor, e.g., a transcriptional regulator, which is capable of regulating a second regulatory region that is operably linked to a second gene, the second regulatory region may be activated or repressed, thereby activating or repressing expression of the second gene. Both a directly inducible promoter and an indirectly inducible promoter are encompassed by inducible promoter.

A promoter can be any polynucleotide that shows transcriptional activity in the chosen host microorganism. A promoter can be naturally-occurring, can be composed of portions of various naturally-occurring promoters, or may be partially or totally synthetic. Guidance for the design of promoters is derived from studies of promoter structure, such as that of Harley and Reynolds, Nucleic Acids Res., 15, 2343-61 (1987). In addition, the location of the promoter relative to the transcription start can be optimized. Many suitable promoters for use in microorganisms and yeast are well known in the art, as are polynucleotides that enhance expression of an associated expressible polynucleotide.

A selectable marker can provide a means to identify microorganisms that express a desired product. Selectable markers include, but are not limited to, ampicillin resistance for prokaryotes such as E. coli, neomycin phosphotransferase, which confers resistance to the aminoglycosides neomycin, kanamycin and paromycin (Herrera-Estrella, EMBO J. 2:987-995, (1983)); dihydrofolate reductase, which confers resistance to methotrexate (Reiss, Plant Physiol. (Life Sci. Adv.) 13:143-149, (1994)); trpB, which allows cells to utilize indole in place of tryptophan; hisD, which allows cells to utilize histinol in place of histidine (Hartman, Proc. Natl. Acad. Sci., USA 85:8047, (1988)); mannose-6-phosphate isomerase which allows cells to utilize mannose (WO 94/20627); hygro, which confers resistance to hygromycin (Marsh, Gene 32:481-485, (1984)); ornithine decarboxylase, which confers resistance to the ornithine decarboxylase inhibitor, 2-(difluoromethyl)-DL-ornithine (DFMO; McConlogue, In: Current Communications in Molecular Biology, Cold Spring Harbor Laboratory ed., (1987)); deaminase from Aspergillus terreus, which confers resistance to Blasticidin S (Tamura, Biosci. Biotechnol. Biochem. 59:2336-2338, (1995)); phosphinothricin acetyltransferase gene, which confers resistance to phosphinothricin (White et al., Nucl. Acids Res. 18:1062, (1990); Spencer et al., Theor. Appl. Genet. 79:625-633, (1990)); a mutant acetolactate synthase, which confers imidazolione or sulfonylurea resistance (Lee et al., EMBO J. 7:1241-1248, (1988)), a mutant EPSPV-synthase, which confers glyphosate resistance (Hinchee et al., BioTechnology 91:915-922, (1998)); a mutant psbA, which confers resistance to atrazine (Smeda et al., Plant Physiol. 103:911-917, (1993)), a mutant protoporphyrinogen oxidase (see U.S. Pat. No. 5,767,373), or other markers conferring resistance to an herbicide such as glufosinate.

A transcription termination region of a recombinant construct or expression cassette is a downstream regulatory region including a stop codon and optionally a transcription terminator sequence. Transcription termination regions that can be used can be homologous to the transcriptional initiation region, can be homologous to the polynucleotide encoding a polypeptide of interest, or can be heterologous (i.e., derived from another source). A transcription termination region or can be naturally occurring, or wholly or partially synthetic. 3′ non-coding sequences encoding transcription termination regions may be provided in a recombinant construct or expression construct and may be from the 3′ region of the gene from which the initiation region was obtained or from a different gene. A large number of termination regions are known and function satisfactorily in a variety of hosts when utilized in both the same and different genera and species from which they were derived. Termination regions may also be derived from various genes native to the preferred hosts. The termination region is usually selected more for convenience rather than for any particular property.

The procedures described herein employ, unless otherwise indicated, conventional techniques of chemistry, molecular biology, microbiology, recombinant DNA, genetics, immunology, cell biology, cell culture and transgenic biology, which are within the skill of the art. (See, e.g., Maniatis, et al., Molecular Cloning, Cold Spring Harbor Laboratory Press, Cold Spring Harbor, N.Y. (1982); Sambrook, et al., (1989); Sambrook and Russell, Molecular Cloning, 3rd Ed., Cold Spring Harbor Laboratory Press, Cold Spring Harbor, N.Y. (2001); Ausubel, et al., Current Protocols in Molecular Biology, John Wiley & Sons (including periodic updates) (1992); Glover, DNA Cloning, IRL Press, Oxford (1985); Russell, Molecular biology of plants: a laboratory course manual, Cold Spring Harbor Laboratory Press, Cold Spring Harbor, N.Y. (1984); Anand, Techniques for the Analysis of Complex Genomes, Academic Press, N Y (1992); Guthrie and Fink, Guide to Yeast Genetics and Molecular Biology, Academic Press, N Y (1991); Harlow and Lane, Antibodies, Cold Spring Harbor Laboratory Press, Cold Spring Harbor, N.Y. (1988); Nucleic Acid Hybridization, B. D. Hames & S. J. Higgins eds. (1984); Transcription And Translation, B. D. Hames & S. J. Higgins eds. (1984); Culture Of Animal Cells, R. I. Freshney, A. R. Liss, Inc. (1987); Immobilized Cells And Enzymes, IRL Press (1986); B. Perbal, A Practical Guide To Molecular Cloning (1984); the treatise, Methods In Enzymology, Academic Press, Inc., NY); Methods In Enzymology, Vols. 154 and 155, Wu, et al., eds.; Immunochemical Methods In Cell And Molecular Biology, Mayer and Walker, eds., Academic Press, London (1987); Handbook Of Experimental Immunology, Volumes I-IV, D. M. Weir and C. C. Blackwell, eds. (1986); Riott, Essential Immunology, 6th Edition, Blackwell Scientific Publications, Oxford (1988); Fire, et al., RNA Interference Technology From Basic Science to Drug Development, Cambridge University Press, Cambridge (2005); Schepers, RNA Interference in Practice, Wiley-VCH (2005); Engelke, RNA Interference (RNAi): The Nuts &Bolts of siRNA Technology, DNA Press (2003); Gott, RNA Interference, Editing, and Modification: Methods and Protocols (Methods in Molecular Biology), Human Press, Totowa, N.J. (2004); and Sohail, Gene Silencing by RNA Interference: Technology and Application, CRC (2004)).

### Recombinant Bacteria

A recombinant, transgenic, or genetically engineered bacteria has been genetically modified from its native state. Thus, “recombinant bacteria” or “recombinant bacterial cell” refers to a bacterium cell that has been genetically modified from the native state. A recombinant bacterium cell can have, for example, nucleotide insertions, nucleotide deletions, nucleotide rearrangements, gene disruptions, recombinant polynucleotides, heterologous polynucleotides, deleted polynucleotides, nucleotide modifications, or combinations thereof introduced into its DNA. These genetic modifications can be present in the chromosome of the bacteria, or on a plasmid in the bacteria. Recombinant cells disclosed herein can comprise exogenous polynucleotides on plasmids. Alternatively, recombinant cells can comprise exogenous polynucleotides stably incorporated into their chromosome.

A heterologous or exogenous polypeptide or polynucleotide refers to any polynucleotide or polypeptide that does not naturally occur or that is not present in the starting target bacterium. For example, a polynucleotide from bacteria that is transformed into a bacterium cell that does naturally or otherwise comprise the bacterial polynucleotide is a heterologous or exogenous polynucleotide. A heterologous or exogenous polypeptide or polynucleotide can be a wild-type, synthetic, or mutated polypeptide or polynucleotide. In an aspect, a heterologous or exogenous polypeptide or polynucleotide is not naturally present in a starting target bacterium and is from a different genus or species than the starting target bacterium.

A homologous or endogenous polypeptide or polynucleotide refers to any polynucleotide or polypeptide that naturally occurs or that is otherwise present in a starting target bacterium. For example, a polynucleotide that is naturally present in a bacterium is a homologous or endogenous polynucleotide. In an aspect, a homologous or endogenous polypeptide or polynucleotide is naturally present in a starting target bacterium.

A recombinant bacterium can comprise one or more polynucleotides not present in a corresponding wild-type cell, wherein the polynucleotides have been introduced into that bacterium using recombinant DNA techniques, or which polynucleotides are not present in a wild-type bacterium and is the result of one or more mutations.

In an aspect a genetically engineered or recombinant bacterium comprises one or more heterologous or exogenous polynucleotides, optionally operably linked to one or more heterologous, exogenous, or endogenous regulatory elements such that one or more heterologous or exogenous biologically active polypeptides are expressed by the bacterium.

The term “overexpression” or “overexpressed” as used herein refers to a level of enzyme or polypeptide expression that is greater than what is measured in a wild-type cell of the same species as the host cell that has not been genetically altered. The overexpression of the enzymes or polypeptides can be achieved by constructing inducible overexpression vectors encoding for the desired polypeptide. Strong promoters can be used to induce overexpression of a polypeptide as can the use of multiple copies of a polynucleotide in the recombinant microorganism. In an aspect a polypeptide of the glcDEF operon or CatA polypeptide, or combinations thereof can be overexpressed.

Pseudomonas putida Group Bacteria

Provided herein are recombinant Pseudomonas putida group bacteria. P. putida group bacteria include Pseudomonas aegrilactucae, Pseudomonas alloputida, Pseudomonas ceruminis, Pseudomonas faucium, Pseudomonas fulva, Pseudomonas huaxiensis, Pseudomonas inefficax, Pseudomonas monteilii, Pseudomonas mosselii, Pseudomonas oryzihabitans, Pseudomonas parasichuanensis, Pseudomonas persica, Pseudomonas plecoglossicida, Pseudomonas putida, Pseudomonas reidholzensis, Pseudomonas shirazica, Pseudomonas syncyanea, Pseudomonas urethralis, and Pseudomonas wadenswilerensis

In aspects Pseudomonas putida group bacteria are genetically engineered using any suitable technique. In some aspects one or more of ped operon, pedD, pedE, pedF, pedG, pedH, pedI, glcR, cat RBCA cluster, catR, catB, catC, catA, tpaAa, tpaAb, tpaB, tpaC, tpaK, glcDEF operon, glcD, glcE, glcF, phaG, alkK, phaC1, phaC2, aroY, and/or an ecdB polynucleotides are deleted, rendered non-functional, or added to P. putida.

### PED Operon

In an aspect a recombinant bacterium, e.g., P. putida group bacterium, has a deleted or non-functional ped operon. The ped operon (PP_2673-PP_2680) includes pedD (UniProt E0X6D7), pedE (UniProt B1N7J0), pedF (UniProt E0X6D9), pedG (UniProt B1N7J2), pedH (UniProt B1N7J5), and pedI (UniProt B1N7J6), which are PQQ-dependent alcohol dehydrogenases.

### TPA Genes

One or more of a large subunit of terephthalate 1,2-dioxygenase (tpaAa), small subunit of terephthalate 1,2-dioxygenase (tpaAb), ferredoxin reductase subunit (tpaB), dihydrodiol dehydrogenase (tpaC), and terephthalate transporter (tpaK) can be genetically engineered into a target bacterium, e.g., a P. putida group bacteria, so that the target bacterium can express a large subunit of terephthalate 1,2-dioxygenase, a small subunit of terephthalate 1,2-dioxygenase, a ferredoxin reductase subunit, dihydrodiol dehydrogenase, and terephthalate transporter. In an aspect, the genes encoding the proteins can be obtained from Rhodococcus jostii or other suitable organism.

A tpAa gene can encode a polypeptide from Rhodococcus jostii (UniProt: QORWE4). In an aspect a tpAa polynucleotide can encode a polypeptide having 80, 85, 90, 95, 96, 97, 98, 99% or more sequence identity to UniProt QORWE4. In other aspects a tpAa gene can encode tpAa polypeptides comprising 80, 85, 90, 95, 96, 97, 98, 99% or more sequence identity to tpAa polypeptides from other Rhodococcus species such as R. oxybenzonivorans (GenBank WP_109335575.1), R. opacus (GenBank WP_182624295.1), R. wratislaviensis (GenBank ELB91386.1), R. spA14 (GenBank WP_167116034.1), R. sp. WB9 (GenBank WP_QDQ95148.1), R. rhodochrous (GenBank WP_192378916.1), R. sp. A5 (2022) (GenBank WP_268966047.1), R. pyridinivorans (GenBank WP_231414406.1), or from other bacteria such as Streptomyces tuiris (GenBank WP_198427730.1), Streptomyces sp. McG7 (GenBank MBT2870189.1), Streptomyces sp. McG8 (GenBank MBT2903652.1), Pseudonocardia bannensis (GenBank WP_211169522.1; NMH90736.1), Pseudonocardia acidicola (GenBank WP_169385232.1), or Actinacidiphila oryziradicis (GenBank WP_211265251.1; TKA12035.1).

A tpAb gene can encode a polypeptide from Rhodococcus jostii (UniProt: QORWE5). In an aspect a tpAb polynucleotide can encode a polypeptide having 80, 85, 90, 95, 96, 97, 98, 99% or more sequence identity to UniProt QORWE5. In other aspects a tpAb gene can encode tpAb polypeptides comprising 80, 85, 90, 95, 96, 97, 98, 99% or more sequence identity to tpAa polypeptides from other Rhodococcus species such as Rhodococcus, GenBank WP_005256013.1, Rhodococcus sp. IEGM 248, GenBank NDV05983.1, Rhodococcus koreensis, GenBank WP_072943899.1, Rhodococcus, GenBank WP_109335576.1, Rhodococcus, GenBank WP_007297312.1, Rhodococcus, GenBank WP_120285013.1, unclassified Rhodococcus, GenBank WP_197251146.1, Rhodococcus, GenBank WP_006552196.1, Rhodococcus pyridinivorans, GenBank WP_193902718.1, Rhodococcus pyridinivorans, GenBank WP_231414405.1, Rhodococcus sp. HM1, GenBank MCK8671711.1, Rhodococcus pyridinivorans, GenBank WP_264721962.1, Rhodococcus pyridinivorans, GenBank WP_232512525.1, or from other bacteria such as Streptomyces sp. Akac8, GenBank WP_026243306.1, Streptomyces sp. McG7, GenBank MBT2870190.1, Actinacidiphila oryziradicis, GenBank TKA12036.1, Streptomyces, GenBank WP_019521463.1, unclassified Streptomyces, GenBank WP_202415819.1, Streptomyces cellulosae, GenBank GHE61500.1, Streptomyces sp. AC04842, GenBank MCP8708519.1, Streptomyces tuirus, GenBank WP_198427729.1, Actinacidiphila oryziradicis, GenBank WP_211265252.1, Streptomyces sp. McG7, GenBank WP_251143999.1, Pseudonocardia acidicola, GenBank NMI01718.1, or Streptomyces sp. GenBank AC04842.

A tpaB gene can encode a polypeptide from Rhodococcus jostii (UniProt: QORWE7). In an aspect a tpaB polynucleotide can encode a polypeptide having 80, 85, 90, 95, 96, 97, 98, 99% or more sequence identity to UniProt QORWE7. In other aspects a tpaB gene can encode tpaB polypeptides comprising 80, 85, 90, 95, 96, 97, 98, 99% or more sequence identity to tpAa polypeptides from other Rhodococcus species such as Rhodococcus koreensis, GenBank WP_072943901.1, Rhodococcus, GenBank WP_043789745.1, Rhodococcus opacus, GenBank WP_182624294.1, Rhodococcus opacus, GenBank WP_037203490.1, Rhodococcus sp. WB9, GenBank WP_144288571.1, Rhodococcus, GenBank WP_007297314.1, Rhodococcus sp. JS3073, GenBank WP_268792611.1, Rhodococcus sp. DK17, GenBank WP_016884993.1, Rhodococcus sp. IEGM 248, GenBank NDV05985.1, Rhodococcus oxybenzonivorans, GenBank WP_109335578.1, Rhodococcus wratislaviensis IFP 2016, GenBank ELB91383.1, Rhodococcus opacus M213, GenBank EKT82360.1, Rhodococcus sp. CX, GenBank WP_197251144.1, Rhodococcus sp. HM1, GenBank WP_248674232.1, Rhodococcus pyridinivorans, GenBank WP_268656030.1, Rhodococcus, GenBank WP_088898935.1, Rhodococcus rhodochrous, GenBank WP_120285017.1, Rhodococcus rhodochrous, GenBank WP_145708850.1, Rhodococcus, GenBank WP_039584281.1, Rhodococcus rhodochrous, GenBank WP 192378918.1, Rhodococcus pyridinivorans, GenBank WP_193902716.1, Rhodococcus pyridinivorans, GenBank WP_094979934.1, Rhodococcus zopfii, GenBank WP_138999838.1, Rhodococcus pyridinivorans, GenBank WP_006552194.1, Rhodococcus pyridinivorans, GenBank WP_231414403.1, Rhodococcus pyridinivorans, GenBank WP_264721963.1, or other bacteria such as Pseudomonas aeruginosa, GenBank HCF1642819.1, Pseudomonas veronii, GenBank WP_232591921.1, Pseudomonas reactans, GenBank NWA45924.1, Stutzerimonas frequens, GenBank WP_208218941.1, Pseudomonas umsongensis, GenBank OXR34351.1, Pseudomonas sp. TH39 (2020), GenBank MBK5395611.1, Pseudomonas sp. Ag1, GenBank EJF73182.1, Pseudomonas sp. TH41, GenBank MBK5351559.1, Streptomyces sp. SID4956, GenBank MYQ35497.1, Streptomyces tuirus, GenBank BCL23960.1, or Pseudomonas putida, GenBank WP_103444219.1.

A tpaC gene can encode a polypeptide from Rhodococcus jostii GenBank ABH00390.1. In an aspect a tpaC polynucleotide can encode a polypeptide having 80, 85, 90, 95, 96, 97, 98, 99% or more sequence identity to GenBank ABH00390.1. In other aspects a tpaC gene can encode tpaC polypeptides comprising 80, 85, 90, 95, 96, 97, 98, 99% or more sequence identity to tpAa polypeptides from other Rhodococcus species such as Rhodococcus sp. DK17, GenBank WP_016884994.1, Rhodococcus, GenBank WP_007297313.1, Rhodococcus koreensis, GenBank WP_072943900.1, Rhodococcus sp. IEGM 248, GenBank NDV05984.1, Rhodococcus, GenBank WP_005256014.1, Rhodococcus opacus, GenBank WP_065492696.1, Rhodococcus opacus, GenBank WP_005566453.1, Rhodococcus sp. WB9, GenBank WP_144288570.1, Rhodococcus, GenBank WP_109335577.1, Rhodococcus sp. DK17, GenBank AAR90189.1, Rhodococcus sp. CX, GenBank WP_197251145.1, Rhodococcus sp. HM1, GenBank WP_248674231.1, Rhodococcus sp. A5 (2022), GenBank WP_268966046.1, Rhodococcus pyridinivorans, GenBank WP_193902717.1, Rhodococcus pyridinivorans, GenBank WP_268656029.1, Rhodococcus, GenBank WP_088898936.1, Rhodococcus pyridinivorans, GenBank WP_231414404.1, Rhodococcus rhodochrous, GenBank WP 159419422.1, Rhodococcus sp. DMU2021, GenBank WP_039584282.1, Rhodococcus pyridinivorans, GenBank WP_006552195.1, Rhodococcus pyridinivorans, GenBank WP_094979933.1, Rhodococcus rhodochrous, GenBank WP_192378917.1, Rhodococcus zopfii, GenBank WP_240795083.1, Rhodococcus, GenBank WP_230792357.1, Rhodococcus, GenBank WP_235367005.1, or Rhodococcus pyridinivorans, GenBank WP_230826506.1.

A tpaK gene can encode a polypeptide from Rhodococcus jostii UniProt QORWE8. In an aspect a tpaK polynucleotide can encode a polypeptide having 80, 85, 90, 95, 96, 97, 98, 99% or more sequence identity to UniProt QORWE8. In other aspects a tpaK gene can encode tpaK polypeptides comprising 80, 85, 90, 95, 96, 97, 98, 99% or more sequence identity to tpAa polypeptides from other Rhodococcus species such as Rhodococcus sp. DK17, GenBank WP_016884992.1, Rhodococcus oxybenzonivorans, GenBank WP_109335579.1, Rhodococcus opacus, GenBank WP_037203491.1, Rhodococcus sp. IEGM 248, GenBank NDV05986.1, Rhodococcus sp. A14, GenBank WP_167116031.1, Rhodococcus opacus, GenBank WP_169692907.1, Rhodococcus opacus, GenBank WP_182624293.1, Rhodococcus sp. WB9, GenBank WP_144288572.1, Rhodococcus opacus RKJ300=JCM 13270, GenBank EID79824.1, Rhodococcus opacus M213, GenBank EKT82361.1, Rhodococcus wratislaviensis IFP 2016, GenBank ELB91382.1, Rhodococcus opacus, GenBank WP_231137766.1, Rhodococcus opacus, GenBank MBA8961580.1, Rhodococcus sp. HM1, GenBank WP_248674233.1, Rhodococcus sp. CX, WP_197251143.1, Rhodococcus pyridinivorans, WP_258912954.1, Rhodococcus, GenBank WP_039584280.1, Rhodococcus, GenBank WP_088898934.1, Rhodococcus aetherivorans, GenBank WP_263510540.1, Rhodococcus pyridinivorans, Gen Bank WP_006552193.1, Rhodococcus pyridinivorans, GenBank WP_231414402.1, Rhodococcus pyridinivorans, GenBank WP_094979935.1, Rhodococcus rhodochrous, GenBank WP_229583249.1, or other bacteria such as Pseudomonas agarici, GenBank WP_017134363.1, or Streptomyces tuirus, GenBank WP_190903364.1.

Repressor Gene of the Glycolate Dehydrogenase (Glc) Pathway (glcR)

A recombinant Pseudomonas putida group bacterium can comprise a deleted or non-functional repressor gene of the glycolate dehydrogenase (Glc) pathway (glcR), which is UniProt A0A379KF86.

glcDEF Operon

A recombinant Pseudomonas putida group bacterium can comprise a recombinant strong constitutive promoter operably linked to a glcDEF operon. The glcDEF operon is comprised of glcD (UniProt A0A616Y085), glcE (UniProt A0A0D1MU47), and glcF (UniProt R9V823). As used herein, a constitutive promoter can provide a continuous transcription process of the desired gene. A strong promoter, as used herein, is a DNA sequence that controls a transcription rate of a gene, which is high for a strong promoter and low for a weak promoter. A strong promoter can have a transcription rate that is 10, 20, 30, 40, 50, 60, 70, 80, 90% or more as compared to a wild-type promoter of a gene. For all instances herein, a strong constitutive promoter can be, e.g., Tac Promoter (P_{tac}). See, e.g., de Boer et al., “The tac promoter: a functional hybrid derived from the trp and lac promoters”. Proceedings of the National Academy of Sciences of the United States of America. 80 (1): 21-25 (1983). A P_{tac} promoter can be optimized the −35/−10 region. The sequence from −35 to −10 can be, for example, TTGACAattaatcatcggctcgTATAAT (SEQ ID NO:93). See, e.g., Elmore et al., Metabolic Engineering Comm. 5 (2017): 1-8. Other strong promoters that can be used include, for example, P4, P6, P9, P16, and P25. See, Zhao et al., Sci Rep 9, 1798 (2019). In an aspect, a recombinant Pseudomonas putida group bacterium can further comprise a recombinant ribosome binding site operably linked to the glcDEF operon. A ribosome binding site can be, e.g., TAAGGAGGT (SEQ ID NO:94). See, e.g., Aparicio et al. Iscience 23.3 (2020): 100946. Other ribosome binding sites include, for example,

SEQ ID NO: 95
GATCAATACGAGCTCATTTCTCCCCGGAGA;
SEQ ID NO: 96
GCGACCTAAGAGATACTAAGTACGAGGTAACAGCC;
SEQ ID NO: 97
TGACCATCCCCCCCTTAAGCGAGGTATTAA;
SEQ ID NO: 98
ATCAAATAAAAAAAGGAGGTAGTCACCCT;
SEQ ID NO: 99
ACCCGTAAATACGGGTCACTATTAGGAGGCATAAG;
SEQ ID NO: 100
CATAAAGAGCTAGATCCTCGGGAGGTCAAGT;
SEQ ID NO: 101
TGAAAAGGACATTCGAGAAGGGGGGTATTT;
SEQ ID NO: 102
AAAGGTTAAGCACAAAAGAGGAGGAGCACT;
SEQ ID NO: 103
TATAATAAGTAATTCTTAAGGGGGTAAGTCC;
SEQ ID NO: 104
TGAGGGGACTACACGTCAAAGGAGGTCGAGA;
SEQ ID NO: 105
AGAATTTTTAGGAGCTAATAGGGAGGTCAGA;
SEQ ID NO: 106
AGAAAGAAAAGAGGAACGAGGAGGTATTTA;
SEQ ID NO: 107
GAAAGAATCCTATCCCAATAAGGAGGTATATT;
SEQ ID NO: 108
TCAAATAATATTAAGCGATAAGGAGGTTTTAG;
SEQ ID NO: 109
CACAGGAAGTACGGTATAAGGAGGTAGGGA.

### PHAG, ALKK, PHAC1, and PHAC2

A recombinant Pseudomonas putida group bacterium can comprise additional copies (e.g., 1, 2, 3 or more) of a hydroxyacyl-ACP thiolase (phaG; PP_1408 UniProt O85207)), a hydroxyacyl-CoA synthase (alkK; PP_0763 UniProt Q9L4M6), and two PHA polymerases (phaC1 and phaC2; PP_5003 UniProt Q7X5K2 and PP_5005 UniProt Q5QBP5), which can be integrated into the chromosome and overexpressed using a strong constitutive promoter (e.g., P_{tac} or other suitable promoter such as a constitutive promoter like Pₑₘ₇). These genes can be codon optimized and/or inserted into the chromosome by replacing a gene such as acetaldehyde dehydrogenase (aldB; PP_0545).

In an aspect a phaG polynucleotide can encode a polypeptide comprising 80, 85, 90, 95, 96, 97, 98, 99% or more sequence identity to PP_1408 UniProt O85207. In an aspect, other phaG polynucleotides can encode phaG polypeptides having 80, 85, 90, 95, 96, 97, 98, 99% or more sequence identity to, e.g., Pseudomonas sp. (GenBank WP_010952516.1), Pseudomonas sp. (GenBank HBM67023.1), Pseudomonas nitroreducens (GenBank AAK71349.1), Pseudomonas putida (GenBank WP_143495074.1), Pseudomonas oleovorans (GenBank AAK81868.1), Pseudomonas mendocina (GenBank AAQ16175.1), Pseudomonas shirazica (GenBank WP_238066874.1), Pseudomonas asiática (GenBank WP_137162308.1), Pseudomonas sp. KCA11 (GenBank WP_233694160.1), Pseudomonas sp. C5pp (GenBank WP_039613742.1), Pseudomonas sp. ABFPK (GenBank WP_061550378.1), Pseudomonas plecoglossicida (GenBank WP_047595792.1), Pseudomonas monteilii (GenBank KXK70351.1), Pseudomonas asiática (GenBank WP_182329027.1), Pseudomonas sp. NY5710 (GenBank WP_173890894.1), or Pseudomonas juntendi (GenBank WP_161891993.1)).

In an aspect a alkK polynucleotide can encode a polypeptide comprising 80, 85, 90, 95, 96, 97, 98, 99% or more sequence identity to Pseudomonas putida (GenBank CAB69080.1). In an aspect, other alkK polynucleotides can encode alkK polypeptides having 80, 85, 90, 95, 96, 97, 98, 99% or more sequence identity to, e.g., Pseudomonas sp. 7-41 GenBank (WP_134939329.1), Pseudomonas veronii (GenBank WP_126588968.1), Halopseudomonas pelagia (GenBank WP_022963141.1), Pseudomonas taeanensis (GenBank WP_029866523.1), unclassified Pseudomonas (GenBank WP_020798868.1), Pseudomonadaceae (GenBank WP_058134802.1), Pseudomonas aeruginosa (GenBank WP_146537847.1), Pseudomonas oleovorans (GenBank Q00594.1), Pseudomonas aeruginosa (GenBank HCF6142133.1), Pseudomonas aeruginosa (GenBank WP_058156749.1), or Pseudomonas aeruginosa (GenBank HBP6436009.1)).

In an aspect a phaC1 polynucleotide can encode a polypeptide comprising 80, 85, 90, 95, 96, 97, 98, 99% or more sequence identity to GenBank AAN70569.1. In an aspect, other phaC polynucleotides can encode phaC1 polypeptides having 80, 85, 90, 95, 96, 97, 98, 99% or more sequence identity to, e.g., Pseudomonas monteilii (GenBank WP_156867872.1), Pseudomonas sp. NBRC 111133 (GenBank WP_060500592.1), Pseudomonas sp. 10-1B (GenBank WP_051099914.1), Pseudomonas oryzicola (GenBank WP_186672340.1), Pseudomonas anuradhapurensis (GenBank WP_186684341.1), Pseudomonas sp. SK (GenBank WP_170033202.1), Pseudomonas plecoglossicida (GenBank WP_102083221.1), Aeromonas caviae (GenBank GJB79526.1), Pseudomonas asiática (GenBank WP_137162561.1), Pseudomonas juntendi (GenBank WP_235768153.1), Pseudomonas guariconensis (GenBank AYA42669.1), or Aeromonas hydrophila (GenBank AAV41872.1).

In an aspect a phaC2 polynucleotide can encode a polypeptide comprising 80, 85, 90, 95, 96, 97, 98, 99% or more sequence identity to GenBank AAN70571.1. In an aspect, other phaC2 polynucleotides can encode phaC2 polypeptides having 80, 85, 90, 95, 96, 97, 98, 99% or more sequence identity to, e.g., other nucleic acid molecules encoding phaC2 (e.g., Pseudomonas monteilii (GenBank WP_063977302.1), Pseudomonas sp. NBRC 111134 GenBank (WP_060516886.1), Pseudomonas sp. NBRC 111141 (GenBank WP_070086473.1), Pseudomonas sp. LRP2-20 (GenBank WP_261942384.1), Pseudomonas sp. NBRC 111123 (GenBank WP_060484671.1), Priestia aryabhattai (GenBank QPN45921.1), Pseudomonas shirazica (GenBank WP_139657889.1) Pseudomonas sp. B5 (2017) (GenBank WP_085663496.1), Pseudomonas plecoglossicida (GenBank WP_108480790.1), Pseudomonas taiwanensis (GenBank WP_186598247.1), Pseudomonas sp. NBRC 111124 (GenBank WP_060509741.1), Pseudomonas monteilii (GenBank TXI07128.1), Aeromonas caviae (GenBank GJB79524.1), or Pseudomonas vlassakiae (GenBank WP_186601728.1). phaG, alkK, phaC1, and phaC2 can be operably linked to one or more strong or constitutive promoters. (e.g., P_{tac} or other suitable promoter such as a constitutive promoter like Pₑₘ₇).

A P. putida group bacterium (e.g., Exemplary Bacteria I; e.g., Pp-T) can comprise a deleted or non-functional ped operon and recombinant nucleic acid molecules encoding a large subunit of terephthalate 1,2-dioxygenase (tpaAa), small subunit of terephthalate 1,2-dioxygenase (tpaAb), ferredoxin reductase subunit (tpaB), dihydrodiol dehydrogenase (tpaC), and terephthalate transporter (tpaK).

A recombinant P. putida group bacterium (e.g., Exemplary Bacteria II, e.g. Pp-E) can comprise a deleted or non-functional repressor gene of the glycolate dehydrogenase (Glc) pathway (glcR), which is UniProt A0A379KF86, and a recombinant strong constitutive promoter operably linked to a glcDEF operon. The glcDEF operon is comprised of glcD (UniProt A0A616Y085), glcE (UniProt A0A0D1MU47), and glcF (UniProt R9V823). A strong constitutive promoter can be, e.g., Tac Promoter (P_{tac}) or other suitable promoter. In an aspect, a recombinant Pseudomonas putida bacterium can further comprise a recombinant ribosome binding site operably linked to the glcDEF operon.

Another aspect comprises a recombinant P. putida group bacterium (e.g., Exemplary Bacteria III, e.g., Pp-TP) comprising a deleted or non-functional ped operon; recombinant nucleic acid molecules encoding tpaAa, tpaAb, tpaB, tpaC, and tpaK; and recombinant nucleic acid molecules encoding hydroxyacyl-ACP acyltransferase (phaG), acyl-CoA-synthase (alkK), mcl-PHA synthase 1 (phaC1) and mcl-PHA synthase 2 (phaC2).

Another aspect comprises a recombinant P. putida group bacterium (e.g., Exemplary Bacteria IV; e.g., PpEP) comprising a deleted or non-functional glcR gene, a recombinant strong constitutive promoter operably linked to a glcDEF operon; and recombinant nucleic acid molecules encoding phaG, alkK, phaC1, and phaC2. A strong constitutive promoter can be, e.g., Tac Promoter (P_{tac}) or other suitable promoter. phaG, alkK, phaC1, and phaC2 can be operably linked to one or more strong or constitutive promoters. (e.g., P tac or Pₑₘ₇).

Another aspect comprises a recombinant P. putida group bacterium (Exemplary Bacteria V, e.g., Pp-TC) comprising a deleted or non-functional ped operon; recombinant nucleic acid molecules encoding tpaAa, tpaAb, tpaB, tpaC, and tpaK, a deleted or non-functional catRBCA cluster; a recombinant nucleic acid molecule encoding PCA decarboxylase (aroY); and a recombinant nucleic acid molecule encoding decarboxylase enhancer (ecdB).

Another aspect comprises a recombinant P. putida group bacterium (e.g. Exemplary Bacteria VI, e.g., Pp-EM) comprising a deleted or non-functional gclR gene; a recombinant strong constitutive promoter operably linked to a glcDEF operon a deleted or non-functional catRBCA cluster; and a recombinant strong constitutive promoter operably linked to a nucleic acid molecule encoding major catechol 1,2-dioxygenase (catA). A strong constitutive promoter can be, e.g., Tac Promoter (P_{tac}) or other suitable promoter.

Another aspect comprises a recombinant P. putida group bacterium (e.g., Exemplary Bacteria VII, e.g., Pp-TAPI) that can comprise a deleted or non-functional ped operon and recombinant nucleic acid molecules encoding a large subunit of terephthalate 1,2-dioxygenase (tpaAa), small subunit of terephthalate 1,2-dioxygenase (tpaAb), ferredoxin reductase subunit (tpaB), dihydrodiol dehydrogenase (tpaC), and terephthalate transporter (tpaK), reduced expression of icd (via, e.g., use of a weak start codon or recombinant weak promoter operably linked to the icd gene in place of the wild type promoter), a recombinant nucleic acid molecule encoding acn, recombinant nucleic acid molecules encoding phaG, alkK, phaC1, and phaC2, which can be operably linked to one or more strong or constitutive promoters (e.g., P tac or other suitable promoter such as a constitutive promoter like Pₑₘ₇), and a recombinant nucleic acid molecule encoding cadA.

Another aspect comprises a recombinant P. putida group bacteria (e.g., Exemplary Bacteria VIII, e.g., Pp-EAPI) that can comprise a can comprise a deleted or non-functional repressor gene of the glycolate dehydrogenase (Glc) pathway (glcR), which is UniProt A0A379KF86, and a recombinant strong constitutive promoter operably linked to a glcDEF operon. The glcDEF operon is comprised of glcD (UniProt A0A616Y085), glcE (UniProt A0A0D1MU47), and glcF (UniProt R9V823). A strong constitutive promoter can be, e.g., Tac Promoter (P_{tac}) or other suitable promoter. In an aspect, a recombinant Pseudomonas putida group bacterium can further comprise a recombinant ribosome binding site operably linked to the glcDEF operon, reduced expression of icd (via, e.g., use of a weak start codon or recombinant weak promoter operably linked to the icd gene in place of the wild type promoter), a recombinant nucleic acid molecule encoding acn, recombinant nucleic acid molecules encoding phaG, alkK, phaC1, and phaC2, which can be operably linked to one or more strong or constitutive promoters (e.g., P tac or other suitable promoter such as a constitutive promoter like Pₑₘ₇), and a recombinant nucleic acid molecule encoding cadA.

In certain aspects 1, 2, 3, 4, or more of the recombinant strains described herein can degrade PET, TPA, and/or EG by about 60, 70, 80, 90, 95, 99% or more within about 10, 20, 30, 40, 50, 60, 70, 80, 90, or more hours.

### Bacterial Cultures for Degrading Polyethylene Terephthalate (PET)

In an aspect a bacterial culture comprising two or more of the recombinant bacteria described herein is provided. For example, a bacterial culture can comprise (1) a recombinant Pseudomonas putida group bacterium comprising a deleted or non-functional ped operon and recombinant nucleic acid molecules encoding a large subunit of terephthalate 1,2-dioxygenase (tpaAa), small subunit of terephthalate 1,2-dioxygenase (tpaAb), ferredoxin reductase subunit (tpaB), dihydrodiol dehydrogenase (tpaC), and terephthalate transporter (tpaK) (e.g., Exemplary Bacterium I) and (2) a recombinant Pseudomonas putida group bacterium comprising a deleted or non-functional repressor gene of the glycolate dehydrogenase (Gcl) pathway (gclR) and a recombinant strong constitutive promoter operably linked to a glcDEF operon (e.g., Exemplary Bacterium II).

### Methods of Degrading Polyethylene Terephthalate (PET)

Polyethylene terephthalate (PET) includes pieces, particles or particulate matters, either in the solid form or in semi solid/viscous form, of any dimensions, primarily composed of polyethylene terephthalate (PET). Examples of polyethylene terephthalate (PET) based polymers or polymeric materials that can be used be includes, but is not limited to, PET based refused products, wastes generated during production of PET products, industrial PET based waste, consumer led PET based plastic wastes such as municipal plastic wastes, PET based plastic wastes dumped at the landfill sites, PET bottle flakes, yarn waste, thermoformed packages, PET fabric, bright PET yam waste popcorn, semi dull PET yam waste popcorn and mixtures thereof. Methods are provided herein for degrading polyethylene terephthalate (PET). PET can be depolymerized by hydrolysis, such as alkaline hydrolysis, to form a hydrolyzed PET product. The alkaline hydrolysis can comprise contacting the PET with about 0.5, 1.0, 2.0, 3.0, 4.0, 5%, or more NaOH. In an aspect, the NaOH solution can be mixed with benzalkonium chloride (BKC). For example, about 0.001, 0.01, 0.05, 0.1 mol of BKC per PET repeating unit can be used. The hydrolysis can take place at about 90, 100, 110, 120, 130, 140, 150 °C. Other hydrolysis methods can be used including, e.g., ammonium hydrolysis, neutral hydrolysis (using water or steam and water-soluble salts as catalysts), and hydrolysis using alkali-metal acetates as effective catalysts. The hydrolyzed PET product can be mixed with a growth medium (e.g., LB medium, M9 minimal medium, M9 medium or any other suitable medium) and bacterial cultures. The hydrolyzed PET product, growth medium, and bacterial cultures can fermented by, for example, a batch, fed-batch, continuous, or other suitable fermentation process.

A hydrolyzed PET product can be a product where 50, 60, 70, 80, 85, 90, 95, 98, 99% or more of the starting PET is depolymerized via hydrolysis to TPA and EG. In an aspect, a hydrolyzed PET product can be a product where 85, 90, 95, 98, 99% or more of the starting PET is depolymerized via hydrolysis to TPA and EG. In an aspect, a hydrolyzed PET product can be a product where 95, 98, 99% or more of the starting PET is depolymerized via hydrolysis to TPA and EG.

The hydrolyzed PET product can be mixed with growth media, which can then be filtered. The hydrolyzed PET product can be added to the growth media at any suitable concentration. In an aspect, a suitable concentration of hydrolyzed PET product can be used at about 20, 30, 40, 50, 60, 70, 80 mM or more. A hydrolyzed PET product can comprise about 30, 40, 50, 60, 70, 80, 90, 100, 150, 200, 250, 300 mM or more of terephthalic acid (TPA). A hydrolyzed PET product can comprise about 30, 40, 50, 60, 70, 80, 90, 100, 150, 200, 250, 300 mM or more of ethylene glycol (EG). A hydrolyzed PET product can comprise about 30, 40, 50, 60, 70, 80, 90, 100, 150, 200, 250, 300 mM or more of terephthalic acid (TPA) and about 30, 40, 50, 60, 70, 80, 90, 100, 150, 200, 250, 300 mM or more of ethylene glycol (EG). The hydrolyzed PET product, growth medium, and bacterial cultures can fermented by, for example, a batch, fed-batch, continuous, or other suitable fermentation process. Fermentation can be for about 100, 72, 36, 48, 24 hours or less.

In an aspect, wherein 50, 60, 70, 80, 90, 95, 98, 99% or more of terephthalic acid (TPA) and 50, 60, 70, 80, 90, 95, 98, 99% or more of ethylene glycol (EG) present in the hydrolyzed PET product is degraded.

Bacterial cultures can comprise (1) a recombinant Pseudomonas putida group bacterium (e.g., Exemplary Bacteria I) comprising a deleted or non-functional ped operon and recombinant nucleic acid molecules encoding a large subunit of terephthalate 1,2-dioxygenase (tpaAa), small subunit of terephthalate 1,2-dioxygenase (tpaAb), ferredoxin reductase subunit (tpaB), dihydrodiol dehydrogenase (tpaC), and terephthalate transporter (tpaK) and (2) a recombinant Pseudomonas putida group bacterium (e.g., Exemplary Bacteria II) comprising a deleted or non-functional repressor gene of the glycolate dehydrogenase (Gcl) pathway (gclR) and a recombinant strong constitutive promoter operably linked to a glcDEF operon. In an aspect, Exemplary Bacteria I is added to the hydrolyzed product and growth media first, and then, after a suitable time period (e.g., about 1, 2, 5, 10, 12, 18, 24, 36 hours or more), Exemplary Bacteria II is added to the hydrolyzed product and growth media. In an aspect, Exemplary Bacteria II is added to the hydrolyzed product and growth media first, and then, after a suitable time period (e.g., about 1, 2, 5, 10, 12, 18, 24, 36 hours or more), Exemplary Bacteria I is added to the hydrolyzed product and growth media. In an aspect, Exemplary Bacteria I and Exemplary Bacteria II are added to the hydrolyzed product and growth media at the same time. In an aspect, Exemplary Bacteria I and Exemplary Bacteria II are present in co-culture and the co-culture added to the hydrolyzed product and growth media.

In an aspect, bacteria, e.g., Exemplary Bacteria I and II, can be added to growth media and hydrolyzed product at a ratio of 1:1, 1:2, 1:3, 1:4, 1:5, 1:6, 2:1, 3:1, 4:1, 5:1 or 6:1, or any other suitable ratio of two bacterial types.

In an aspect TPA and EG can be degraded 60, 70, 80, 90, 95, 99% or more within about 10, 20, 30, 40, 50, 60, 70, 80, 90, or more hours.

### Bacteria for Making Medium Chain Length Polyhydroxyalkanoates

Certain aspects include a recombinant Pseudomonas putida group bacterium comprising a deleted or non-functional ped operon and recombinant nucleic acid molecules encoding tpaAa, tpaAb, tpaB, tpaC, and tpaK. The recombinant Pseudomonas putida group bacterium can also comprise additional copies (e.g., 1, 2, 3 or more) of a hydroxyacyl-ACP thiolase (phaG), a hydroxyacyl-CoA synthase (alkK), and two PHA polymerases (phaC1 and phaC2), which can be integrated into the chromosome and overexpressed using a strong constitutive promoter (e.g., P tac or other suitable promoter) (e.g., Exemplary Bacterium III). phaG, alkK, phaC1, and phaC2 can be operably linked to one or more strong or constitutive promoters (e.g., P tac or other suitable promoter such as a constitutive promoter like Pₑₘ₇). These genes can be codon optimized and/or inserted into the chromosome by replacing a gene such as acetaldehyde dehydrogenase (aldB; PP_0545).

Another aspect comprises a recombinant Pseudomonas putida group bacterium comprising a deleted or non-functional glcR gene, a recombinant strong constitutive promoter operably linked to a glcDEF operon; and recombinant nucleic acid molecules encoding phaG, alkK, phaC1, and phaC2 (e.g., Exemplary Bacteria IV).

### Bacteria for Making Medium Chain Length Polyhydroxyalkanoates

In an aspect, a bacterial culture comprising two or more of the recombinant bacteria described herein is provided. For example, a bacterial culture can comprise (1) a recombinant Pseudomonas putida group bacterium comprising: (i) a deleted or non-functional ped operon; (ii) recombinant nucleic acid molecules encoding tpaAa, tpaAb, tpaB, tpaC, and tpaK; and (iii) recombinant nucleic acid molecules encoding hydroxyacyl-ACP acyltransferase (phaG), acyl-CoA-synthase (alkK), mcl-PHA synthase 1 (phaC1) and mcl-PHA synthase 2 (phaC2); and (2) a recombinant Pseudomonas putida group bacterium comprising: (i) a deleted or non-functional gclR gene; (ii) a recombinant strong constitutive promoter operably linked to a glcDEF operon; and (iii) recombinant nucleic acid molecules encoding phaG, alkK, phaC1 and phaC2.

### Methods of Making Medium Chain Length Polyhydroxyalkanoates

An aspect provides methods of making medium chain length polyhydroxyalkanoates (mcl-PHA (C6 to C14, (e.g., C6, C7, C8, C9, C10, C11, C12, C13, or C14)) comprising depolymerizing PET by alkaline hydrolysis to form a hydrolyzed PET product (as described above), mixing the hydrolyzed product with a growth medium (e.g., LB medium, M9 minimal medium, M9 medium or any other suitable medium), and adding a bacterial culture to a mixture of the hydrolyzed product and the growth medium, wherein mcl-PHA (C6-C14) is produced. About 100, 200, 300 or more mg/L of mcl-PHA can be produced. The hydrolyzed PET product, growth medium, and bacterial cultures can fermented by, for example, a batch, fed-batch, continuous, or other suitable fermentation process. The medium chain length polyhydroxyalkanoates can be collected and purified.

The alkaline hydrolysis can comprise contacting the PET with about 0.5, 1.0, 2.0, 3.0, 4.0, 5%, or more NaOH. Other hydrolysis methods can be used including, e.g., ammonium hydrolysis, neutral hydrolysis (using water or steam and water-soluble salts as catalysts), and hydrolysis using alkali-metal acetates as effective catalysts. The bacterial culture can comprise (1) a recombinant Pseudomonas putida group bacterium comprising: (i) a deleted or non-functional ped operon; (ii) recombinant nucleic acid molecules encoding tpaAa, tpaAb, tpaB, tpaC, and tpaK; and (iii) recombinant nucleic acid molecules encoding hydroxyacyl-ACP acyltransferase (phaG), acyl-CoA-synthase (alkK), mcl-PHA synthase 1 (phaC1) and mcl-PHA synthase 2 (phaC2); and (2) a recombinant Pseudomonas putida group bacterium comprising: (i) a deleted or non-functional gclR gene; (ii) a recombinant strong constitutive promoter operably linked to a glcDEF operon; and (iii) recombinant nucleic acid molecules encoding phaG, alkK, phaC1 and phaC2.

In an aspect, a recombinant Pseudomonas putida group bacteria such as Exemplary Bacteria III can be added to the hydrolyzed product and growth media first, and then, after a suitable time period (e.g., about 1, 2, 5, 10, 12, 18, 24, 36 hours or more), a recombinant Pseudomonas putida group bacteria such as Exemplary Bacteria IV can be added to the hydrolyzed product and growth media. In an aspect, a recombinant Pseudomonas putida group bacteria such as Exemplary Bacteria IV can be added to the hydrolyzed product and growth media first, and then, after a suitable time period (e.g., about 1, 2, 5, 10, 12, 18, 24, 36 hours or more), a recombinant Pseudomonas putida group bacteria such as Exemplary Bacteria III can be added to the hydrolyzed product and growth media. In an aspect, a recombinant Pseudomonas putida group bacteria such as Exemplary Bacteria III and a recombinant Pseudomonas putida group bacteria such as Exemplary Bacteria IV are added to the hydrolyzed product and growth media at the same time. In an aspect, a recombinant Pseudomonas putida group bacteria such as Exemplary Bacteria III and a recombinant Pseudomonas putida group bacteria such as Exemplary Bacteria IV are present in co-culture and the co-culture added to the hydrolyzed product and growth media.

In an aspect recombinant Pseudomonas putida group bacteria e.g., Exemplary Bacteria III and IV, can be added to growth media and hydrolyzed product at a ratio of 1:1, 1:2, 1:3, 1:4, 1:5, 1:6, 1:7, 1:8, 1:9, 2:1, 3:1, 4:1, 5:1, 6:1, 7:1, 8:1, 9:1 or any other suitable ratio of two bacterial types.

Recombinant Pseudomonas putida Group Bacterium for MA Production

Another aspect comprises a recombinant Pseudomonas putida group bacterium comprising: (i) a deleted or non-functional ped operon; (ii) recombinant nucleic acid molecules encoding tpaAa, tpaAb, tpaB, tpaC, and tpaK, (iii) a deleted or non-functional catRBCA cluster; (iv) a recombinant nucleic acid molecule encoding PCA decarboxylase (aroY) GenBank e.g., ADF61496.1; WP_100168896.1; WP_320724810.1; WP_130626125.1; and (v) a recombinant nucleic acid molecule encoding decarboxylase enhancer (ecdB) (e.g., Exemplary Bacteria V).

In an aspect an ecdB polynucleotide can be encoded by a polypeptide comprising 80, 85, 90, 95, 96, 97, 98, 99% or more sequence identity to an ecdB polynucleotide from Enterobacter cloacae ATCC 13047, e.g., GenBank ADF63617.1

In an aspect, other ecdB polynucleotides can encode ecdB polypeptides having 80, 85, 90, 95, 96, 97, 98, 99% or more sequence identity to e.g., Enterobacter hormaechei (GenBank WP_045308748.1), Enterobacter cloacae complex (GenBank WP_023323673.1), Klebsiella pneumoniae (HBL90019.1), Pedobacter himalayensis (GenBank OOK73268.1), Enterobacter quasihormaechei (GenBank WP_131636299.1), Enterobacter asburiae (GenBank WP_211137934.1), or Enterobacter wuhouensis (GenBank WP_263610301.1).

A deleted or non-functional catRBCA cluster can comprise catR (UniProt P20667), catB (UniProt P08310), catC (UniProt P95539), and catA (UniProt Q59714).

Another aspect comprises a recombinant Pseudomonas putida group bacterium comprising: a deleted or non-functional gclR gene; a recombinant strong constitutive promoter operably linked to a glcDEF operon; a deleted or non-functional catRBCA cluster; and a recombinant strong constitutive promoter (e.g. P_{tac} or other suitable bacteria) operably linked to a nucleic acid molecule encoding major catechol 1,2-dioxygenase (catA) (e.g., Exemplary Bacteria 6). In an aspect a CatA polynucleotide can encode a polypeptide comprising 80, 85, 90, 95, 96, 97, 98, 99% or more sequence identity to (e.g., UniProt Q6XUN7). In an aspect, other CatA polynucleotides can encode CatA polypeptides having 80, 85, 90, 95, 96, 97, 98, 99% or more sequence identity to other nucleic acid molecules encoding CatA (e.g., Pseudomonas putida (GenBank CAB69080.1), Pseudomonas putida (GenBank WP_011154379.1; WP_121783457.1; WP_180688083.1; WP_061203275.1; WP_119713612.1); Pseudomonas azerbaijanoriens (GenBank WP_217835190.1), Pseudomonas sp. GM33 (GenBank WP_007980495.1), Pseudomonas fluorescens (GenBank WP_019689164.1),

Pseudomonas fluorescens (GenBank WP_057397862.1; WP_150787653.1), Pseudomonas sp. ES3-33 (GenBank WP_045058475.1), Pseudomonas sp. PD9R (GenBank WP_218437873.1), Pseudomonas sp. GV071 (GenBank WP_108094836.1), Pseudomonas thermotolerans (GenBank WP_017939038.1), Pseudomonas saudiphocaensis (GenBank WP_037022063.1), Pseudomonas carbonaria (GenBank WP_187673145.1)).

Another aspect comprises a bacterial culture comprising (1) a recombinant Pseudomonas putida group bacterium comprising: (i) a deleted or non-functional ped operon; (ii) recombinant nucleic acid molecules encoding tpaAa, tpaAb, tpaB, tpaC, and tpaK, (iii) a deleted or non-functional catRBCA cluster; (iv) a recombinant nucleic acid molecule encoding PCA decarboxylase (aroY); and (v) a recombinant nucleic acid molecule encoding decarboxylase enhancer (ecdB) (e.g., Exemplary Bacteria V) and (2) a recombinant Pseudomonas putida group bacterium comprising: a deleted or non-functional gclR gene; a recombinant strong constitutive promoter operably linked to a glcDEF operon; a deleted or non-functional catRBCA cluster; and a recombinant strong constitutive promoter operably linked to a nucleic acid molecule encoding major catechol 1,2-dioxygenase (catA) (e.g., Bacteria VI).

### Methods of Making Muconate (MA)

An aspect comprises methods of making muconate (MA) comprising depolymerizing PET by alkaline hydrolysis to form a hydrolyzed PET product (as described above), mixing the hydrolyzed product with a growth medium (e.g., LB medium, M9 minimal medium, M9 medium or any other suitable medium), and adding the recombinant Pseudomonas putida group bacteria described above to a mixture of the hydrolyzed product and the growth medium, wherein MA is produced. The hydrolyzed PET product, growth medium, and bacterial cultures can fermented by, for example, a batch, fed-batch, continuous, or other suitable fermentation process. Muconate can be collected and purified. A yield of about 20, 30, 40, 50, 60, 70 mM or more of MA can be achieved. The alkaline hydrolysis can comprise contacting the PET with about 0.5, 1.0, 2.0, 3.0, 4.0, 5%, or more NaOH. Other hydrolysis methods can be used including, e.g., ammonium hydrolysis, neutral hydrolysis (using water or steam and water-soluble salts as catalysts), and hydrolysis using alkali-metal acetates as effective catalysts. The recombinant Pseudomonas putida group bacteria can be Exemplary Bacteria V and VI. In an aspect, Exemplary Bacteria V is added to the hydrolyzed product and growth media first, and then, after a suitable time period (e.g., about 1, 2, 5, 10, 12, 18, 24, 36 hours or more), Exemplary Bacteria VI is added to the hydrolyzed product and growth media. In an aspect, Exemplary Bacteria VI is added to the hydrolyzed product and growth media first, and then, after a suitable time period (e.g., about 1, 2, 5, 10, 12, 18, 24, 36 hours or more), Exemplary Bacteria V is added to the hydrolyzed product and growth media. In an aspect, Exemplary Bacteria V and Exemplary Bacteria VI are added to the hydrolyzed product and growth media at the same time. In an aspect, Exemplary Bacteria V and Exemplary Bacteria VI are present in co-culture and the co-culture added to the hydrolyzed product and growth media.

In an aspect bacteria, e.g., Exemplary Bacteria V and VI, can be added to growth media and hydrolyzed product at a ratio of 1:1, 1:2, 1:3, 1:4, 1:5, 1:6, 1:7, 1:8, 1:9, 2:1, 3:1, 4:1, 5:1, 6:1, 7:1, 8:1, 9:1 or any other suitable ratio of two bacterial types.

Bacteria for Simultaneous mcl-PHA and Itaconic Acid Production.

In an aspect a bacterial culture comprising two or more of the recombinant bacteria described herein is provided. For example, a bacterial culture can comprise (1) a recombinant P. putida group bacterium (e.g., Exemplary Bacteria VII Pp-TAPI) that can comprise a deleted or non-functional ped operon and recombinant nucleic acid molecules encoding a large subunit of terephthalate 1,2-dioxygenase (tpaAa), small subunit of terephthalate 1,2-dioxygenase (tpaAb), ferredoxin reductase subunit (tpaB), dihydrodiol dehydrogenase (tpaC), and terephthalate transporter (tpaK), reduced expression of icd (via, e.g., use of a weak start codon or recombinant weak promoter operably linked to the icd gene in place of the wild type promoter), a recombinant nucleic acid molecule encoding acn, recombinant nucleic acid molecules encoding phaG, alkK, phaC1, and phaC2, which can be operably linked to one or more strong or constitutive promoters (e.g., P tac or other suitable promoter such as a constitutive promoter like Pₑₘ₇), and a recombinant nucleic acid molecule encoding cadA; and (2) a recombinant P. putida group bacterium (e.g., Exemplary Bacteria VIII, e.g., Pp-EAPI) that can comprise a deleted or non-functional repressor gene of the glycolate dehydrogenase (Glc) pathway (glcR), and a recombinant strong constitutive promoter operably linked to a glcDEF operon. A strong constitutive promoter can be, e.g., Tac Promoter (P_{tac}) or other suitable promoter. In an aspect, a recombinant Pseudomonas putida group bacterium can further comprise a recombinant ribosome binding site operably linked to the glcDEF operon, reduced expression of icd (via, e.g., use of a weak start codon or recombinant weak promoter operably linked to the icd gene in place of the wild type promoter), a recombinant nucleic acid molecule encoding acn, recombinant nucleic acid molecules encoding phaG, alkK, phaC1, and phaC2, which can be operably linked to one or more strong or constitutive promoters (e.g., P tac or other suitable promoter such as a constitutive promoter like Pₑₘ₇), and a recombinant nucleic acid molecule encoding cadA.

Expression of icd can be reduced in a recombinant P. putida group bacterium. The start codon of P. putida group isocitrate dehydrogenase (icd) (e.g., GenBank AAN69605.1) can be changed to a weak start codon (e.g., from ATG to GTG) to reduce expression. Other methods of reducing expression of icd, including deletion of the wild-type promoter and replacing it with a weak promoter (such as BG28, BG21, BG15, BG37, BG6, BG11, BG14, BG35, BG25 of Zobel et al., ACS Synth. Biol. 2015, 4, 12, 1341-135) or inducible expression systems can also be used to reduce expression of icd. A variety of native (Pₘ, Pₛₐₗ, P_{alkB}, Pᵤ, and P_{xylA}) and heterologous (P_{araB}, P_{rhaB}, P_{trc}, P_{lac}, P_{tac}, Pₜₑₜ, P_{T7-lac} and P_{lacUV5}, P_{mekA}, P_{mtlE}, P_{chnB}, and P_{DB3}) inducible expression systems can be used in Pseudomonas putida group strains. These induction systems can use a wide range of inducing chemicals such as 3-methylbenzyl alcohol, 3-methylbenzoate, dicyclopropylketone, methyl ethyl ketone, m-toluate, salicylate, n-octane, 3-chloro-4-hydroxyphenylacetic acid, cyclohexanone, rhamnose, arabinose, xylose, mannitol, p-cumate, anhydrotetracycline, and isopropyl-β-D-1-thiogalactopyranoside (IPTG). Carbon source-inducible and substrate-inducible promoter systems, such as HexR/P_{zwf1} induced by glucose, LvaR/P_{lvaA} induced by LA or 4HV, HpdR/P_{hpdH} induced by 3HP or LA, MmsR/P_{mmsA} induced by 3HP or LA, and XutR/P_{xutA} induced by xylose can also be used to reduce expression of icd. See Sathesh-Prabu et al., Inducible and tunable gene expression systems for Pseudomonas putida KT2440. Sci Rep. 2021 Sep. 10; 11 (1): 18079. In an aspect the expression of idc is reduced by about 20, 30, 40, 50, 60, 70% or more as compared to wild-type expression.

A recombinant nucleic acid molecule encoding a heterologous aconitate hydratase gene (acn) from Corynebacterium glutamicum strain ATCC 13032 (UniProt Q8NQ98) can be added to a recombinant P. putida group bacteria In an aspect, a recombinant nucleic acid molecule encoding heterologous aconitate hydratase gene (acn) can be added to recombinant P. putida group bacteria and can comprise a polynucleotide encoding a polypeptide having 70, 75, 80, 85, 90, 95, 98, 99% or more homology to UniProt Q8NQ98, or GenBank numbers WP_186465257.1, WP_186458850.1, TWS38944.1, WP_200877131.1, WP_172769609.1, KIH73519.1, WP_172796479.1, WP_196769292.1, WP_197491730.1, BAF54590.1, AIK85209.1, ALP50228.1, WP_181244864.1, PST75985.1, WP_173673820.1, OKX78491.1, ANE04063.1, WP_247775243.1, WP_225213658.1, NLZ58147.1, WP_232009525.1, WP 206423885.1, WP_257058468.1, WP_046204166.1, WP_047240248.1, WP_029975573.1, WP_233486518.1, WP_202981969.1, WP 180956354.1, WP_237722558.1, WP_227000381.1, WP_231587382.1, and AFM07420.3, AMN70039.1

In an aspect, a recombinant P. putida group (or other suitable bacterium can comprise a cis-aconitic acid decarboxylase gene (cadA) from Aspergillus terreus (UniProt A0A2S1ZAU5). In an aspect, a recombinant nucleic acid molecule encoding heterologous cis-aconitic acid decarboxylase gene (cadA) can be added to a recombinant P. putida group bacterium and can comprise a polynucleotide encoding a polypeptide having 70, 75, 80, 85, 90, 95, 98, 99% or more homology to UniProt A0A2S1ZAU5 or GenBank numbers AAD34563.1, XP_001209273.1, UDM55691.1, AJE61287.1, ALI59896.1, ASR91696.1, ASR91697.1, XP_031894140.1, or KAB8072425.1.

### Methods for Simultaneous Mcl-PHA and Itaconic Acid Production

An aspect comprises methods for simultaneous mcl-PHA and itaconic acid production comprising depolymerizing PET by alkaline hydrolysis to form a hydrolyzed product (as described above), mixing the hydrolyzed product with a growth medium (e.g., LB medium, M9 minimal medium, M9 medium or any other suitable medium), and adding the recombinant Pseudomonas putida group bacteria described above to a mixture of the hydrolyzed product and the growth medium. The hydrolyzed PET product, growth medium, and bacterial cultures can fermented by, for example, a batch, fed-batch, continuous, or other suitable fermentation process. mcl-PHA and itaconic acid can be collected and optionally purified after fermentation. About 100, 150, 200, 250, 300 mg/L of mcl-PHA and about 0.2, 0.5, 1.0, 2.0, 5.0 mM or more of IA can be produced. The alkaline hydrolysis can comprise contacting the PET with about 0.5, 1.0, 2.0, 3.0, 4.0, 5%, or more NaOH. Other hydrolysis methods can be used including, e.g., ammonium hydrolysis, neutral hydrolysis (using water or steam and water-soluble salts as catalysts), and hydrolysis using alkali-metal acetates as effective catalysts. The recombinant Pseudomonas putida group bacteria can be Exemplary Bacteria VII and VIII. In an aspect, recombinant Pseudomonas putida group bacteria, e.g., Exemplary Bacteria V can be added to the hydrolyzed product and growth media first, and then, after a suitable time period (e.g., about 1, 2, 5, 10, 12, 18, 24, 36 hours or more), recombinant Pseudomonas putida group bacteria, e.g., Exemplary Bacteria VIII can be added to the hydrolyzed product and growth media. In an aspect, recombinant Pseudomonas putida group bacteria, e.g., Exemplary Bacteria VIII can be added to the hydrolyzed product and growth media first, and then, after a suitable time period (e.g., about 1, 2, 5, 10, 12, 18, 24, 36 hours or more), recombinant Pseudomonas putida group bacteria, e.g., Exemplary Bacteria VII can be added to the hydrolyzed product and growth media. In an aspect, recombinant Pseudomonas putida group bacteria, e.g., Exemplary Bacteria VII and recombinant Pseudomonas putida group bacteria, e.g., Exemplary Bacteria VII can be added to the hydrolyzed product and growth media at the same time. In an aspect, recombinant Pseudomonas putida group bacteria, e.g., Exemplary Bacteria VII and recombinant Pseudomonas putida group bacteria, e.g., Exemplary Bacteria VIII can be present in co-culture and the co-culture added to the hydrolyzed product and growth media.

In an aspect recombinant Pseudomonas putida group bacteria, e.g., Exemplary Bacteria VII and VIII, can be added to growth media and hydrolyzed product at a ratio of 1:1, 1:2, 1:3, 1:4, 1:5, 1:6, 1:7, 1:8, 1:9, 2:1, 3:1, 4:1, 5:1, 6:1, 7:1, 8:1, 9:1 or any other suitable ratio of two bacterial types.

### Deleted or Non-Functional Polynucleotides

In several aspects the bacteria described herein have has a deleted or non-functional ped operon, a deleted or non-functional repressor gene of the glycolate dehydrogenase (Glc) pathway (glcR), a deleted or non-functional catRBCA cluster catR, catB, catC, and catA.

Deleted, non-functional, or eliminated gene or polynucleotide expression can be gene or polynucleotide expression that is eliminated or reduced to an amount that is insignificant or undetectable. Deleted, non-functional, or eliminated gene or polynucleotide expression can also be gene or polynucleotide expression that results in an RNA or protein that is nonfunctional, for example, deleted gene or polynucleotide expression can be gene or polynucleotide expression that results in a truncated RNA and/or polypeptide that has substantially no biological activity.

In an aspect, a genetically engineered or recombinant microorganism has no expression of a polynucleotide encoding a ped operon, a pedD polypeptide, a pedE polypeptide, a pedF polypeptide, a pedG polypeptide, a pedH polypeptide, a pedI polypeptide, a glcR polypeptide, a catRBCA cluster, a catR polypeptide, a catB polypeptide, a catC polypeptide, a catA polypeptide, or combinations thereof. The result is that substantially no pedD polypeptides, pedE polypeptides, pedF polypeptides, pedG polypeptides, pedH polypeptides, pedI polypeptides, glcR polypeptides, catR polypeptides, catB polypeptides, catC polypeptides, catA polypeptides, or combinations are present in the cell. In an aspect, a genetically engineered or recombinant microorganism has 50, 60, 70, 80, 90, 95, 99% or less expression of a polynucleotide encoding a ped operon, a pedD polypeptide, a pedE polypeptide, a pedF polypeptide, a pedG polypeptide, a pedH polypeptide, a pedI polypeptide, a glcR polypeptide, a catRBCA cluster, a catR polypeptide, a catB polypeptide, a catC polypeptide, a catA polypeptide, or combinations thereof as compared to a wild-type microorganism.

The lack of expression can be caused by at least one gene disruption or mutation of a ped operon, pedD gene, pedE gene, pedF gene, pedG gene, pedH gene, pedI gene, glcR gene, catRBCA cluster, catR gene, catB gene, catC gene, catA gene, or combinations thereof, which results in no expression of the pedD gene, pedE gene, pedF gene, pedG gene, pedH gene, pedI gene, glcR gene, catR gene, catB gene, catC gene, catA gene or combinations thereof. For example, the lack of expression can be caused by a gene disruption in a ped operon, pedD gene, pedE gene, pedF gene, pedG gene, pedH gene, pedI gene, glcR gene, catRBCA cluster, catR gene, catB gene, catC gene, catA gene or combinations thereof which results in attenuated or eliminated expression of the pedD gene, pedE gene, pedF gene, pedG gene, pedH gene, pedI gene, glcR gene, catRBCA cluster, catR gene, catB gene, catC gene, catA gene or combinations thereof such that the genes, operons or clusters can be transcribed but not translated, or the genes can be transcribed and translated, but the resulting pedD polypeptide, pedE polypeptide, pedF polypeptide, pedG polypeptide, pedH polypeptide, pedI polypeptide, glcR polypeptide, catR polypeptide, catB polypeptide, catC polypeptide, catA polypeptide, or combinations thereof, have substantially no biological activity.

In an aspect, a recombinant microorganism is mutated or otherwise genetically altered such that there is substantially no expression of pedD polypeptides, pedE polypeptides, pedF polypeptides, pedG polypeptides, pedH polypeptides, or pedI polypeptides in the cell. In an aspect, a recombinant microorganism is mutated or otherwise genetically altered such that there is substantially no expression of glcR polypeptides thereof in the cell. In an aspect, a recombinant microorganism is mutated or otherwise genetically altered such that there is substantially no expression of catR polypeptides, catB polypeptides, catC polypeptides, catA polypeptides, or combinations thereof in the cell.

In an aspect, a genetically engineered or recombinant microorganism has 50, 60, 70, 80, 90, 95, 99% or less expression of a pedD polypeptide, a pedE polypeptide, a pedF polypeptide, a pedG polypeptide, a pedH polypeptide, a pedI polypeptide, a glcR polypeptide, a catRBCA cluster, a catR polypeptide, a catB polypeptide, a catC polypeptide, a catA polypeptide, or combinations thereof as compared to a wild-type microorganism.

The polynucleotides encoding a ped operon, catRBCA cluster, pedD polypeptide, pedE polypeptide, pedF polypeptide, pedG polypeptide, pedH polypeptide, pedI polypeptide, glcR polypeptide, catR polypeptide, catB polypeptide, catC polypeptide, catA polypeptide, or combinations thereof can be deleted or mutated using any suitable genetic manipulation technique selected from, for example, TALEN, Zinc Finger Nucleases, and CRISPR-Cas9.

One or more regulatory elements controlling expression of the polynucleotides encoding a ped operon, catRBCA cluster, pedD polypeptide, pedE polypeptide, pedF polypeptide, pedG polypeptide, pedH polypeptide, pedI polypeptide, glcR polypeptide, catR polypeptide, catB polypeptide, catC polypeptide, catA polypeptide, or combinations thereof can be mutated or replaced to prevent or attenuate expression of a pedD polypeptide, pedE polypeptide, pedF polypeptide, pedG polypeptide, pedH polypeptide, pedI polypeptide, glcR polypeptide, catR polypeptide, catB polypeptide, catC polypeptide, catA polypeptide, or combinations thereof, as compared to a control or wild-type microorganism. For example, a promoter can be mutated or replaced such that the gene expression or polypeptide expression is attenuated or such that the ped operon, pedD gene, pedE gene, pedF gene, pedG gene, pedH gene, pedI gene, glcR gene, catRBCA cluster, catR gene, catB gene, catC gene, catA gene are not transcribed. In one aspect, one or more promoters for ped operon, pedD gene, pedE gene, pedF gene, pedG gene, pedH gene, pedI gene, glcR gene, catRBCA cluster, catR gene, catB gene, catC gene, catA gene, or combinations thereof are replaced with a promoter that has weaker activity than the wild-type promoter. A promoter with weaker activity transcribes the polynucleotide at a rate about 5, 10, 20, 30, 40, 50, 60, 70, 80, or 90% less than the wild-type promoter for that polynucleotide. In another aspect, one or more promoters for ped operon, pedD gene, pedE gene, pedF gene, pedG gene, pedH gene, pedI gene, glcR gene, catRBCA cluster, catR gene, catB gene, catC gene, catA gene or combinations thereof are replaced with an inducible promoter (e.g., TetO promoters such as TetO3, TetO7, and CUP1p) that can be controlled to attenuate expression of the ped operon, pedD gene, pedE gene, pedF gene, pedG gene, pedH gene, pedI gene, glcR gene, catRBCA cluster, catR gene, catB gene, catC gene, catA gene, or combinations thereof.

The reduced expression, non-expression, or expression of mutated, inactive, or reduced activity polypeptides can be affected by deletion of the polynucleotide or gene encoding pedD polypeptide, pedE polypeptide, pedF polypeptide, pedG polypeptide, pedH polypeptide, pedI polypeptide, glcR polypeptide, catR polypeptide, catB polypeptide, catC polypeptide, and/or catA polypeptide, replacement of the wild-type polynucleotide or gene with mutated forms, deletion of a portion of a ped operon, pedD gene, pedE gene, pedF gene, pedG gene, pedH gene, pedI gene, glcR gene, catRBCA cluster, catR gene, catB gene, catC gene, catA gene or genes or combinations thereof to cause expression of an inactive form of the polypeptides, or manipulation of the regulatory elements (e.g. promoter) to prevent or reduce expression of wild-type pedD polypeptide, pedE polypeptide, pedF polypeptide, pedG polypeptide, pedH polypeptide, pedI polypeptide, glcR polypeptide, catR polypeptide, catB polypeptide, catC polypeptide, and/or catA polypeptides. The promoter could also be replaced with a weaker promoter or an inducible promoter that leads to reduced expression of the polypeptides. Any method of genetic manipulation that leads to a lack of, or reduced expression and/or activity of pedD polypeptide, pedE polypeptide, pedF polypeptide, pedG polypeptide, pedH polypeptide, pedI polypeptide, glcR polypeptide, catR polypeptide, catB polypeptide, catC polypeptide, and/or catA polypeptide and can be used, including expression of inhibitor RNAs (e.g. shRNA, siRNA, and the like).

Wild-type refers to a microorganism that is naturally occurring or which has not been recombinantly modified to increase or decrease transport or utilization of specific sugars. A control microorganism is a microorganism that lacks genetic modifications of a test microorganism and that can be used to test altered biological activity of genetically modified microorganisms.

A gene disruption is a genetic alteration in a polynucleotide or gene that renders an encoded gene product (e.g., pedD polypeptide, pedE polypeptide, pedF polypeptide, pedG polypeptide, pedH polypeptide, pedI polypeptide, glcR polypeptide, catR polypeptide, catB polypeptide, catC polypeptide, and/or catA polypeptide) inactive or attenuated (e.g., produced at a lower amount, e.g. about 50, 60, 70, 80, 90, 95, 99% or more lower amount as compared to wild-type or having lower biological activity e.g. about 50, 60, 70, 80, 90, 95, 99% or more lower biological activity as compared to wild-type). A gene disruption can include a disruption in a polynucleotide or gene that results in no expression of an encoded gene product, reduced expression of an encoded gene product, or expression of a gene product with reduced or attenuated biological activity. The genetic alteration can be, for example, deletion of the entire gene or polynucleotide, deletion of a regulatory element required for transcription or translation of the polynucleotide or gene, deletion of a regulatory element required for transcription or translation or the polynucleotide or gene, addition of a different regulatory element required for transcription or translation or the gene or polynucleotide, deletion of a portion (e.g. 1, 2, 3, 6, 9, 21, 30, 60, 90, 120 or more nucleic acids) of the gene or polynucleotide, which results in an inactive or partially active gene product, replacement of a gene's promoter with a weaker promoter, replacement or insertion of one or more amino acids of the encoded protein to reduce its activity, stability, or concentration, or inactivation of a gene's transactivating factor such as a regulatory protein. A gene disruption can include a null mutation, which is a mutation within a gene or a region containing a gene that results in the gene not being transcribed into RNA and/or translated into a functional gene product. An inactive gene product has no biological activity.

Zinc-finger nucleases (ZFNs), Talens, and CRISPR-Cas9 allow double strand DNA cleavage at specific sites in yeast chromosomes such that targeted gene insertion or deletion can be performed (Shukla et al., 2009, Nature 459:437-441; Townsend et al., 2009, Nature 459:442-445). This approach can be used to modify the promoter of endogenous genes or the endogenous genes themselves to modify expression of pedD polypeptide, pedE polypeptide, pedF polypeptide, pedG polypeptide, pedH polypeptide, pedI polypeptide, glcR polypeptide, catR polypeptide, catB polypeptide, catC polypeptide, and/or catA polypeptide, which can be present in the bacterial genome. ZFNs, Talens or CRISPR/Cas9 can be used to change the sequences regulating the expression of the polypeptides to increase or decrease the expression or alter the timing of expression beyond that found in a non-engineered or wild-type yeast, or to delete the wild-type polynucleotide, or replace it with a deleted or mutated form to alter the expression and/or activity pedD polypeptide, pedE polypeptide, pedF polypeptide, pedG polypeptide, pedH polypeptide, pedI polypeptide, glcR polypeptide, catR polypeptide, catB polypeptide, catC polypeptide, and/or catA polypeptide.

The consortium methods described herein involve, in some aspects, two recombinant Pseudomonas putida group strains, specializing in terephthalic acid and ethylene glycol utilization respectively, to achieve complete degradation. Compared with its monoculture counterpart, the consortium exhibits reduced catabolic crosstalk and faster deconstruction, particularly when substrate concentrations are high or crude hydrolysate is used. It also outperforms monoculture when polyhydroxyalkanoates serves as a target product, confers flexible tuning for cis-cis muconate synthesis, and enables simultaneous polyhydroxyalkanoates and itaconic acid co-production. This work demonstrates engineered consortia as a promising, effective platform that can facilitate polymer upcycling and environmental sustainability.

The compositions and methods are more particularly described below and the Examples set forth herein are intended as illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined herein to provide additional guidance to the practitioner regarding the description of the compositions and methods.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The aspects illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by aspects and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

The following are provided for exemplification purposes only and are not intended to limit the scope of the aspects described in broad terms above.

### EXAMPLES

### Example 1 Construction of a Synthetic Microbial Consortium for TPA and EG Co-Degradation

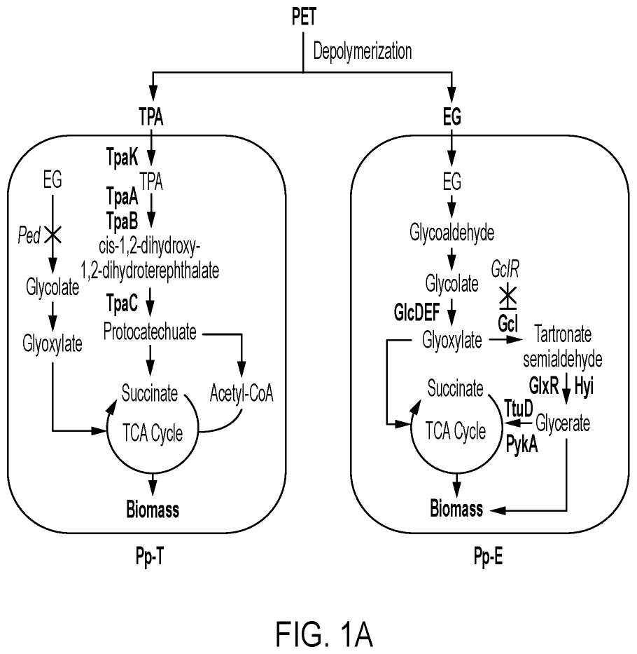

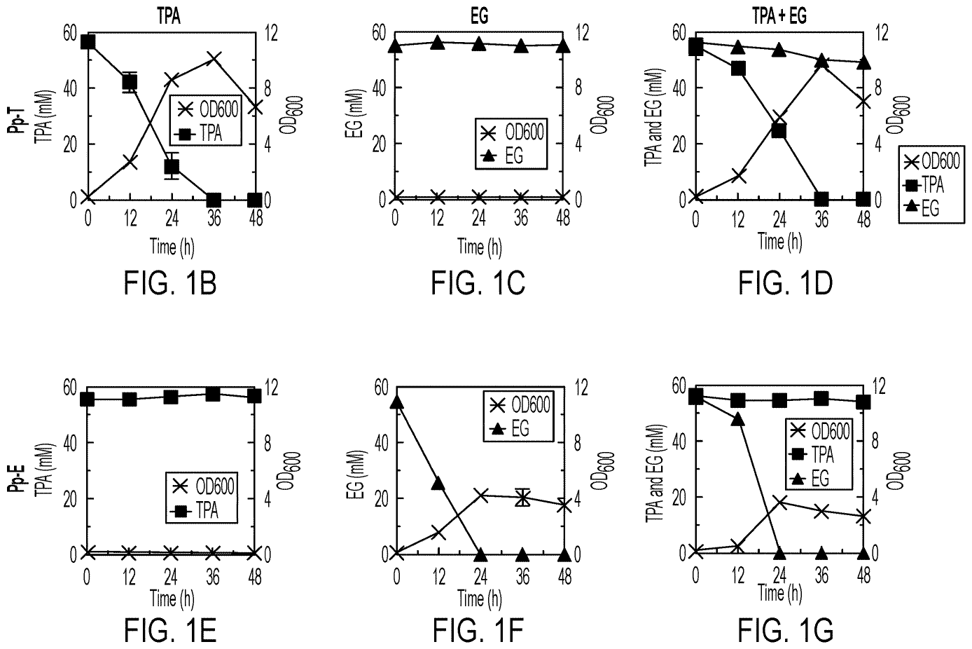

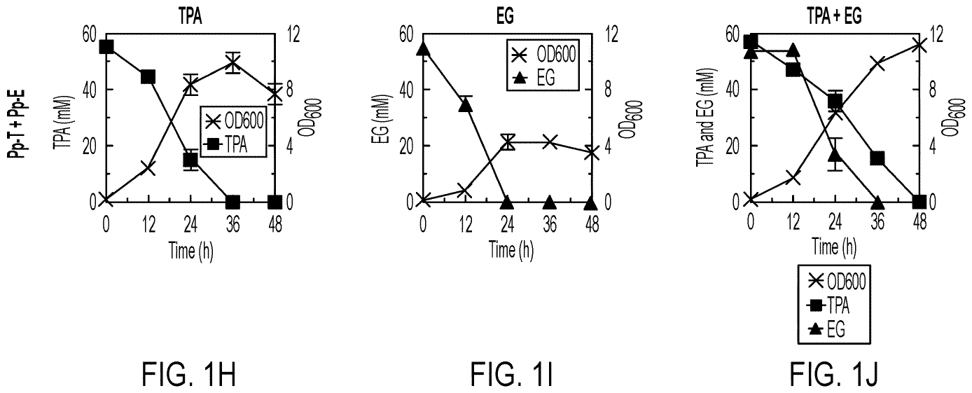

We started by building a P. putida consortium involving two strains that specialize in TPA and EG degradation, respectively (FIG. 1A). The TPA specialist (Pp-T) was developed from the EM42 strain by abolishing its EG assimilation and enabling TPA consumption. The former was achieved by deleting the entire ped gene cluster from the EM42 genome to eliminate its EG oxidation for glycolate generation. The latter was implemented by introducing into the chassis a tpa cluster from Rhodococcus jostii RHA1, which encodes the large and small subunits of terephthalate 1,2-dioxygenase (tpaAa and tpaAb), a reductase component (tpaB), a dihydrodiol dehydrogenase (tpaC) and a transporter (tpaK), to confer the conversion of TPA to protocatechuate (PCA) for strain utilization. The EG specialist (Pp-E) was constructed from M31, an EG-assimilating strain derived from EM42, by deleting the transcriptional repressor gene (gclR) of the Gcl pathway^{13,36} to enhance the carbon flux from glyoxylate to pyruvate, and replacing the native promoter and ribosomal binding site (RBS) of the glycolate oxidase (glcDEF) operon with a strong constitutive promoter (P_{tac}) and an artificial RBS (TAAGGAGGT SEQ ID NO: 94) to overcome glycolate accumulation. See Elmore et al., Metab Eng Commun 5, 1-8 (2017). We hypothesized that such a DOL design would yield catabolically orthogonal specialists that individually consume TPA or EG but collectively enable simultaneous TPA and EG degradation when co-cultured as an ecosystem (T-E consortium).

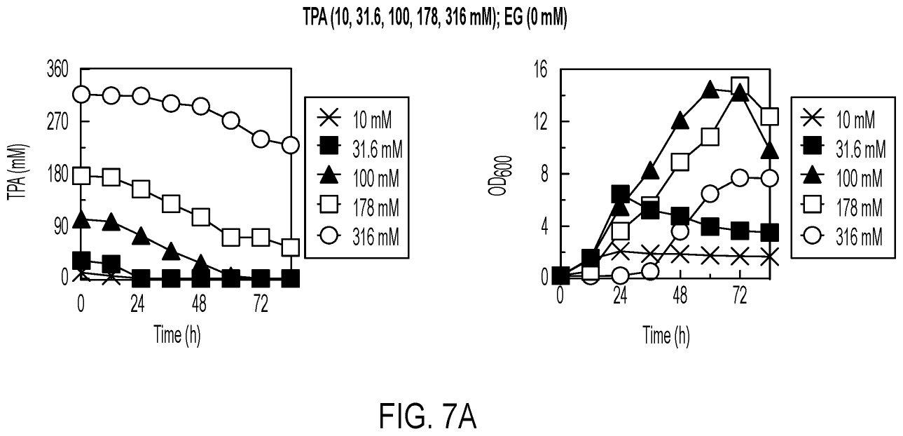

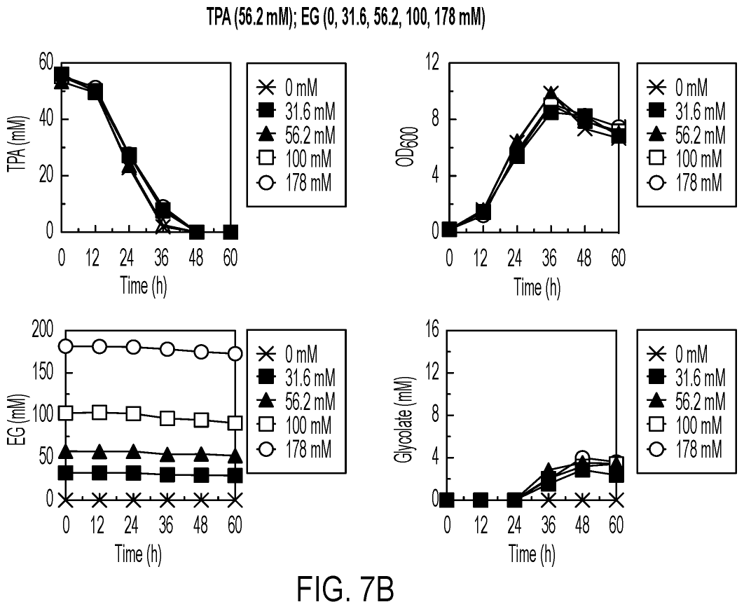

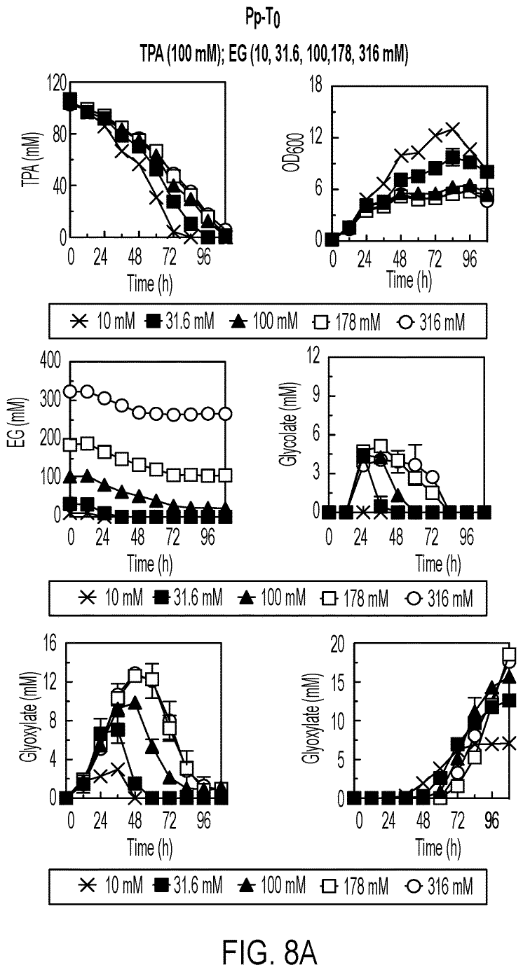

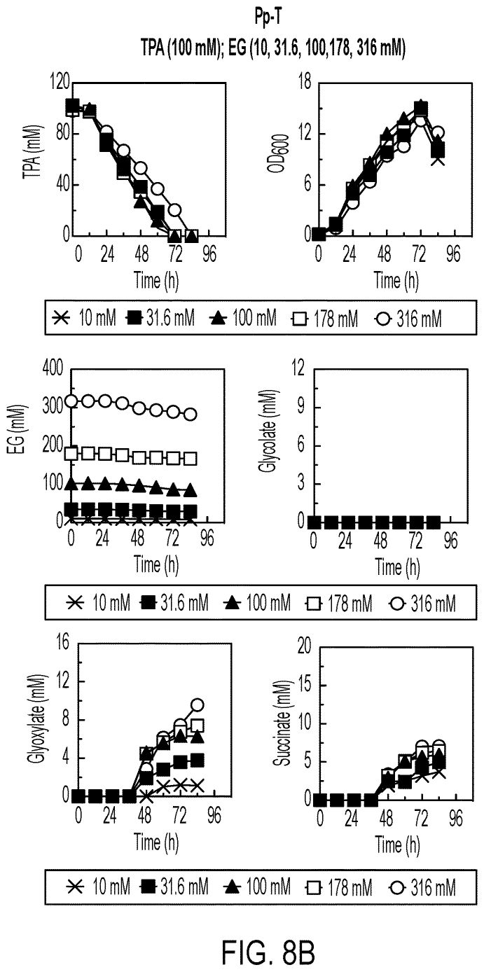

To test this hypothesis and validate the consortium, we performed batch fermentations using TPA, EG and their mix as substrates. Indeed, Pp-T successfully utilized TPA as the sole carbon source for growth and depleted 56 mM TPA within 36 h (FIG. 1B). The strain also completely consumed at least 100 mM TPA and grew in the presence of 316 mM TPA, suggesting a high level of TPA tolerance (FIG. 7A). Meanwhile, Pp-T did not degrade EG when it served as a sole substrate (FIG. 1C). When both TPA and EG were present, the former was fully utilized in 36 h while the latter remained largely unchanged (FIG. 1D and FIG. 7B), confirming that Pp-T specializes in TPA degradation. Notably, unpinning this specialization is our deletion of the ped cluster responsible for EG oxidation. To confirm the functional role of the specialization, we compared the growth, TPA consumption, and intermediate metabolites of Pp-T and its parental strain, Pp-T₀, which possesses the native ped cluster and heterologous tpa cluster (FIG. 8). We found Pp-T remained efficient in TPA degradation and biomass accumulation with the increase of EG concentrations for a given TPA level (100 mM) as compared to Pp-T₀ which was sensitive to the EG concentration. Accordingly, Pp-T yielded profiles of related metabolites different from Pp-T₀. These results suggested that enhancing the specialization of TPA degradation in Pp-T greatly reduces the catabolite interference caused by EG. Thus, Pp-T showed encouraging performance metrics with respect to TPA degradation and catabolic orthogonality.

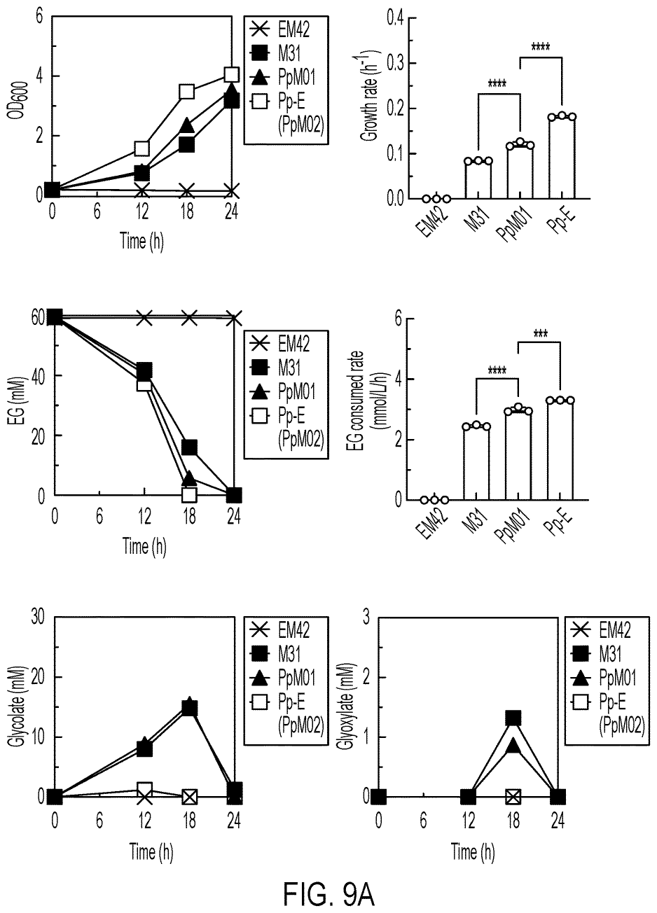

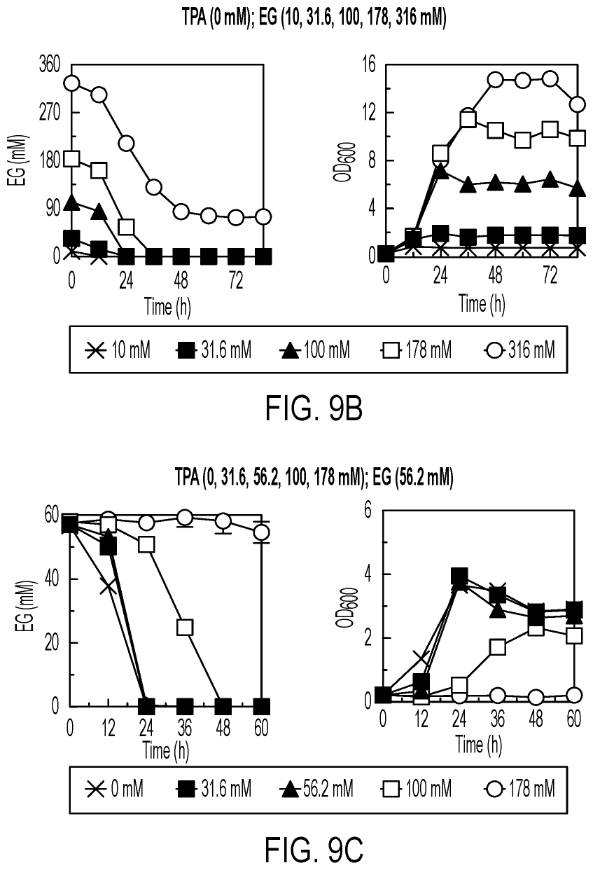

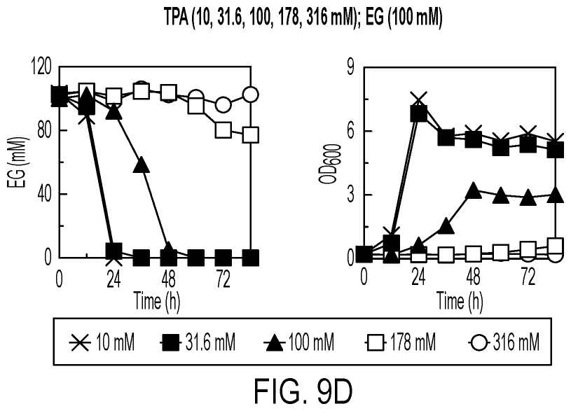

On the other hand, Pp-E did not degrade TPA (FIG. 1E), but was effective in utilizing EG as a sole carbon source (FIG. 1F). Compared to its parental strains, Pp-E was greatly improved in growth and EG consumption along with a negligible intermediate accumulation (FIG. 9A). Pp-E also tolerated a high level of EG (FIG. 9B) and exhibited a high specificity in EG consumption when TPA and EG were both supplied (FIG. 1G). Of note, Pp-E's specialization in EG degradation and orthogonality to TPA consumption is attributed to the fact that Pp-E does not have a TPA assimilation pathway. Meanwhile, for a given EG level (e.g., 56.2 or 100 mM), the EG degradation and biomass growth of Pp-E both decreased and were eventually abolished with the increase of TPA (FIG. 9C, 9D), indicating that Pp-E is sensitive to TPA, different from Pp-T which is tolerant to EG (FIG. 7B).

We also examined the co-culture of Pp-T and Pp-E for substrate utilization. In the presence of TPA or EG alone, the consortium showed similar fermentation patterns as those of the Pp-T and Pp-E monocultures (FIG. 1H, 1I). However, when TPA and EG were both supplied, the consortium efficiently and fully degraded both substrates along with significant biomass production in 48 h (FIG. 1J), distinct from either of the monocultures (FIG. 1D, 1G). The results demonstrate our successful development of the TPA and EG co-utilization consortium.

### Example 2. Comparison of the T-E Consortium and its Single-Strain Counterpart for Substrate Co-Utilization

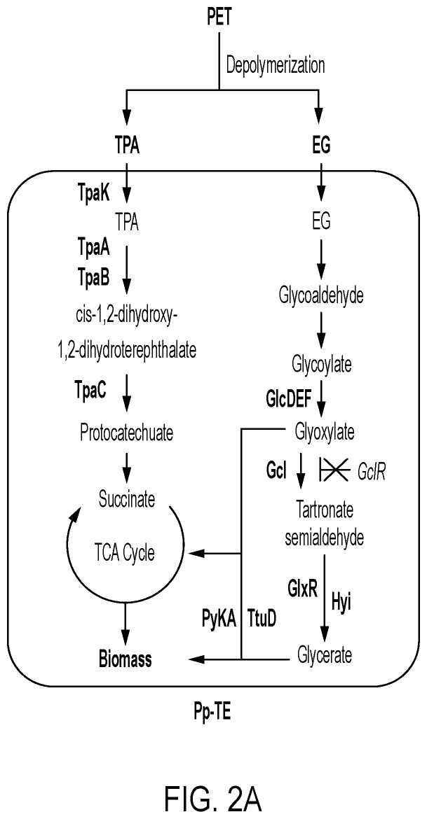

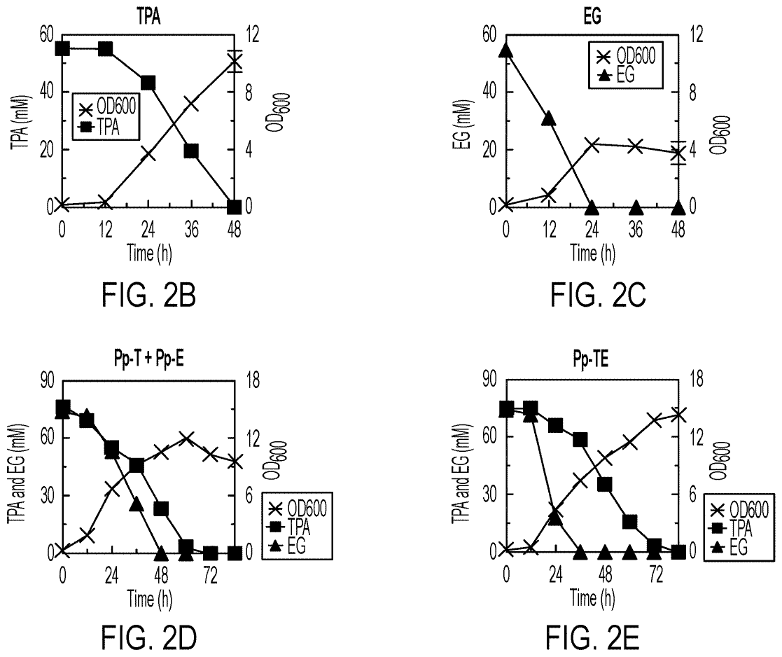

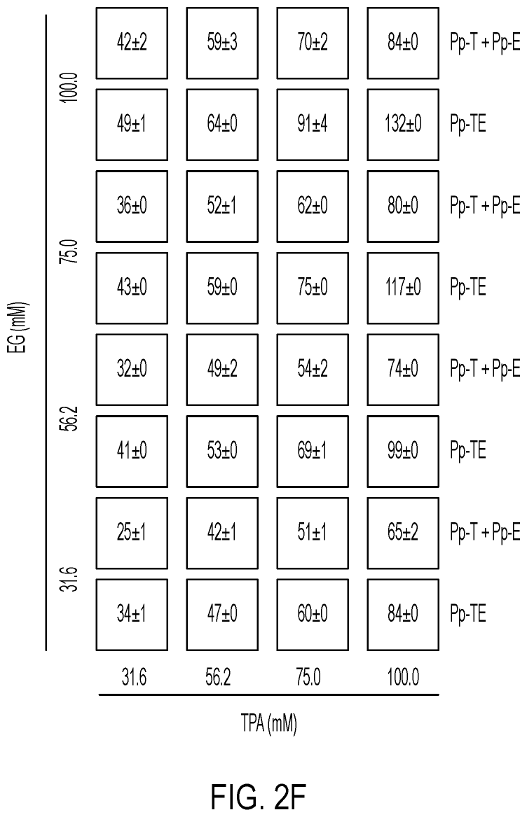

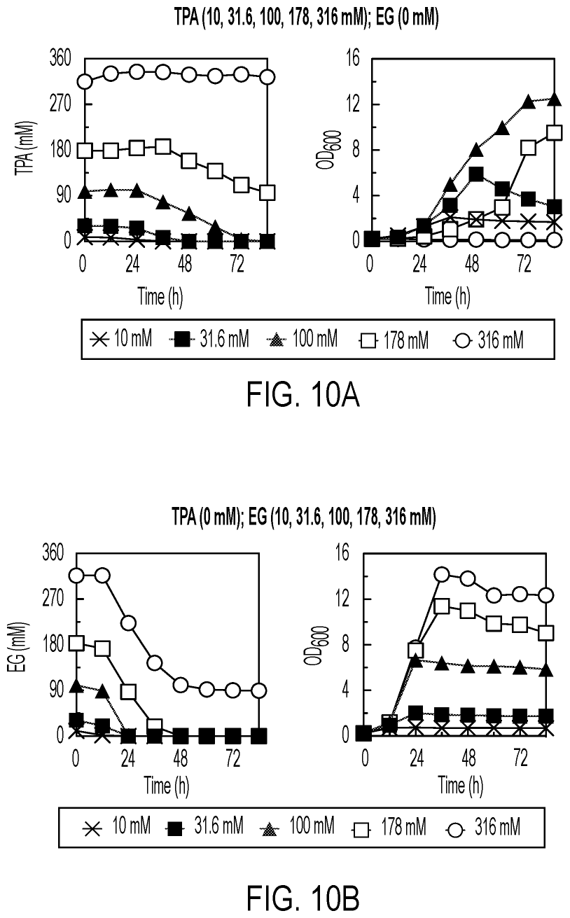

To determine whether co-culture could be superior to monoculture for plastic deconstruction, we engineered a P. putida strain (Pp-TE) that degrades both TPA and EG (FIG. 2A). Here, the dual degradability was programmed by loading the tpa cluster, used for the TPA specialist Pp-T, into the EG specialist Pp-E. Subsequent experiments confirmed that the resulting strain, Pp-TE, was able to grow on TPA or EG as a sole substrate (FIG. 2B,2C). However, compared with Pp-T (FIG. 1B), Pp-TE was slower in TPA-to-biomass conversion and took 48 h to deplete 56 mM TPA (FIG. 2B). To examine if this is a generic trait of Pp-TE, we carried out single-substrate fermentations with various substrate concentrations. We found that the lag time increased systematically with the TPA level and, at 316 mM, the TPA consumption fully stopped (FIG. 10A), suggesting that Pp-TE has a lower TPA tolerance than Pp-T (FIG. 7A). By contrast, a high level of EG did not impair Pp-TE for EG assimilation (FIG. 10B), similar to the case of Pp-E (FIG. 9B)

We next assessed the single utilizer, Pp-TE, and the T-E consortium with mixed TPA and EG fermentations. FIG. 2D, 2E shows that, while the two systems both completely degraded the two substrates (75 mM each), it cost the T-E consortium a much shorter time (60 h) than Pp-TE (72 h). Thus, designing the specialist pair allowed us to enhance the overall degradation of the mixed substrates. Additionally, we noticed a difference in degradation kinetics: TPA and EG were consumed simultaneously by the T-E consortium; by contrast, in the case of Pp-TE, EG was rapidly utilized whereas TPA assimilation exhibited a significant delay that was alleviated only once the EG was mostly exhausted. The latter was phenomenologically similar to sequential consumption in mixed sugar fermentation caused by carbon catabolite repression.

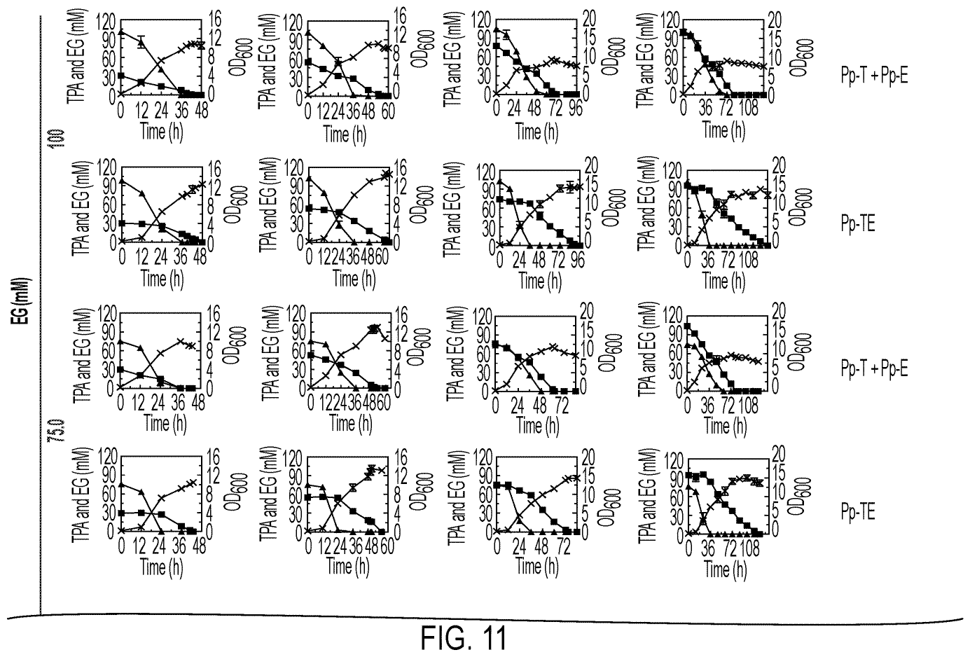

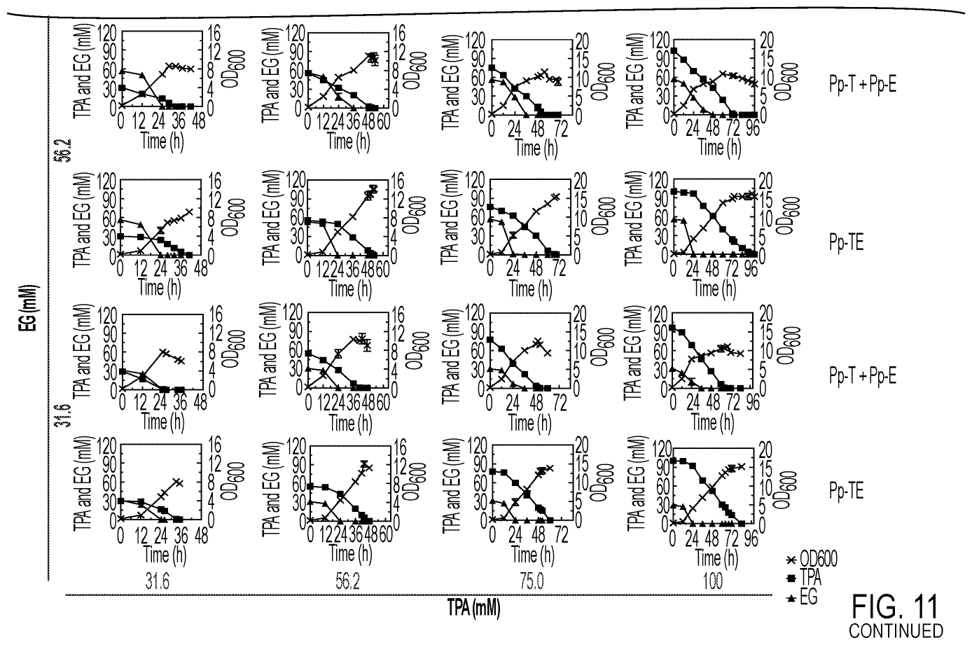

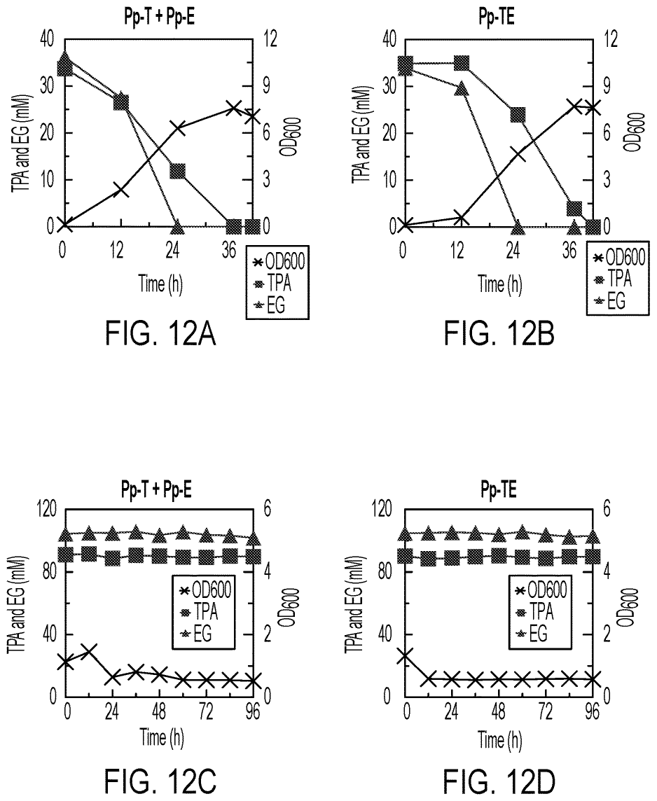

To ascertain the universality of the finding, we expanded the scope of comparison by systematically examining the fermentation times and patterns of the T-E consortium and Pp-TE over 16 substrate combinations. As shown in FIG. 2F and FIG. 12, although the two systems both successfully degraded TPA and EG, the T-E consortium showed a systematic advantage in processing the mixture, especially when TPA alone or both substrates were abundant. For example, Pp-TE took 132 h to exhaust a mixture of 100 mM TPA and 100 mM EG but, for the consortium, it took only 84 h; in this setting, the sequential degradation by Pp-ET also became more noticeable in contrast to simultaneous co-degradation by the T-E consortium (the upper right panels in FIG. 11). The higher degradation efficiency of the consortium may be attributed to two points. First, by compartmentalizing the TPA and EG degradation pathways, the T-E consortium has reduced catabolic cross-interactions between the pathways. Second, by assigning the pathways to separate strains, Pp-T and Pp-E each specialize in degrading one substrate without the need to compromise their specialty as the generalist Pp-TE, and simultaneously carry a lower metabolic burden. Thus, we demonstrated that the T-E consortium is an effective platform for TPA and EG co-consumption empowered by the engineered DOL.

### Example 3 Complete PET Degradation Through Chemical Hydrolysis and Microbial Consumption

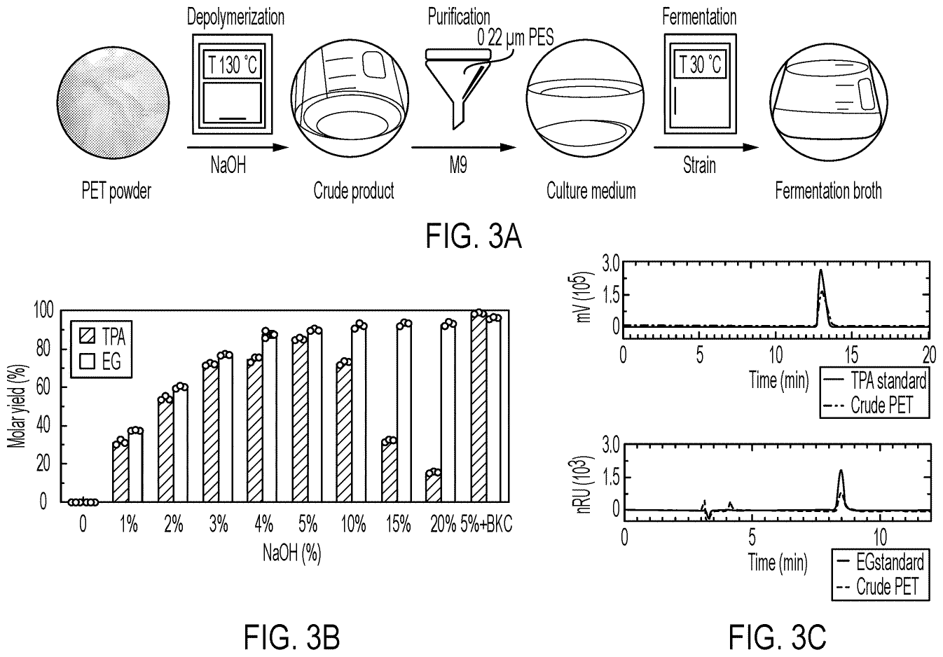

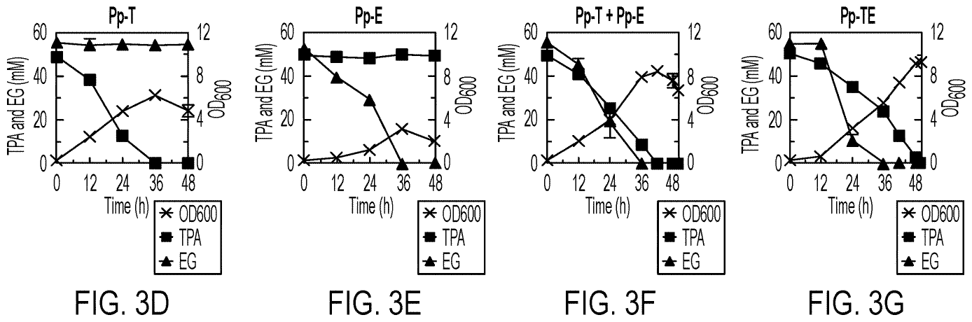

To apply our synthetic consortium for PET degradation, we proposed an integrated workflow that combines chemical hydrolysis with microbial deconstruction (FIG. 3A). Specifically, the workflow involves three steps. First, PET is depolymerized via alkaline hydrolysis. Second, the hydrolyzed product is mixed with M9 medium and filtered to form a growth medium. Third, the medium is inoculated with engineered microbes to fulfill fermentations. Although different strategies are available, we chose ester bonds hydrolysis with alkaline (NaOH) as the catalyst, because it does not require complex processing conditions, and an autoclave reactor is sufficient for performing the hydrolysis. In addition, the resulting products, disodium terephthalate (NazTPA, TPA salt) and EG, can be directly used for biodegradation without extra procedures for acidification, separation, and purification.

To identify an optimal hydrolysis setting, we varied the NaOH concentration from 0 to 20% (FIG. 3B). Without NaOH (0%), the treatment did not result in any PET depolymerization. The Na₂TPA and EG yields both increased with NaOH when its concentration was below 5%. However, when the concentration exceeded 5%, the Na₂TPA yield declined with the alkaline although the EG yield continued to rise minorly, likely due to the salting out of Na₂TPA in the presence of excess NaOH and a high level of EG. Adopting 5% as the NaOH concentration, we further optimized the process by adding benzalkonium chloride (BKC) as phase transfer catalyst, resulting in >97% of final yields for both Na₂TPA and EG. Using HPLC, we directly confirmed that the product was indeed composed of Na₂TPA and EG (FIG. 3C).

We proceeded to perform fermentations using the crude product (~50 mM TPA/EG) from the above hydrolysis. FIG. 3D shows that Pp-T consumed TPA without degrading EG in the hydrolysate, in a rate comparable to the case of the pure TPA and EG mixture (FIG. 1D). Similarly, Pp-E depleted EG in the crude product (FIG. 3E); however, its rate was slower than the pure TPA-EG fermentation (FIG. 1G), suggesting the sensitivity of Pp-E to the hydrolysate. Additionally, we compared the fermentations by the T-E consortium and Pp-TE for different levels of the PET hydrolysate. At both the 31.6 mM (FIG. 12A, 12B) and 56.2 mM (FIG. 3F, 3G) hydrolysate concentrations, the T-E consortium outperformed Pp-TE in terms of fermentation completion time, consistent with the observations for the pure mixture case (FIG. 2F and FIG. 11). Meanwhile, at the 100 mM condition (FIG. 12C, 12D), neither the consortium nor the single strain was able to survive and function, which is distinct from the pure mixture case whereby deconstruction was completed by the T-E consortium and Pp-TE in 84 h and 132 h, respectively (FIG. 2F). Again, this difference suggested that PET hydrolysate had increased toxicity compared to the pure TPA and EG mixture. Collectively, we established an integrated deconstruction process from powdered plastic to microbial biomass whereby the engineered consortium exhibited a compelling capability for substrate conversion.

### Example 4 Upcycling of PET Hydrolysate into Mcl-PHA

To achieve upcycling, we further engineered our microbial consortia by designing and integrating metabolic pathways that upgrade the hydrolysate into valuable products. Specifically, we selected mcl-PHA, ranging from C6 to C14, as our target product because mcl-PHA is one of the most promising biodegradable polymers and our chassis bacterium P. putida has been shown to produce the chemical from different substrates including sugars, organic acids, and aromatic compounds.

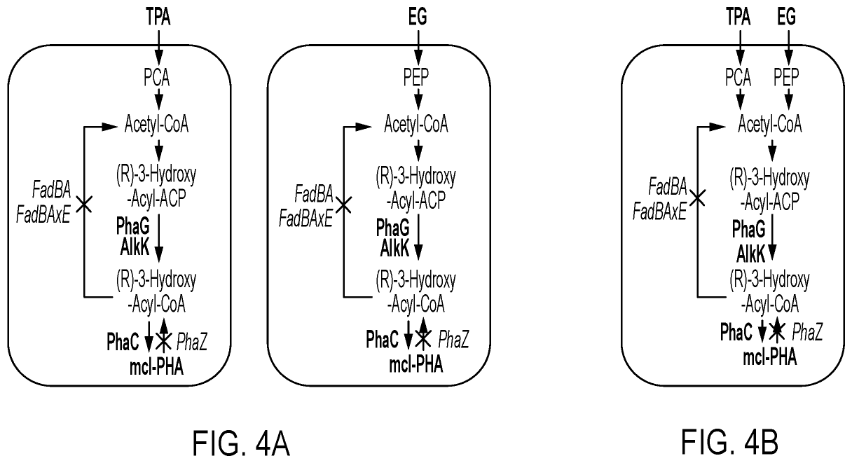

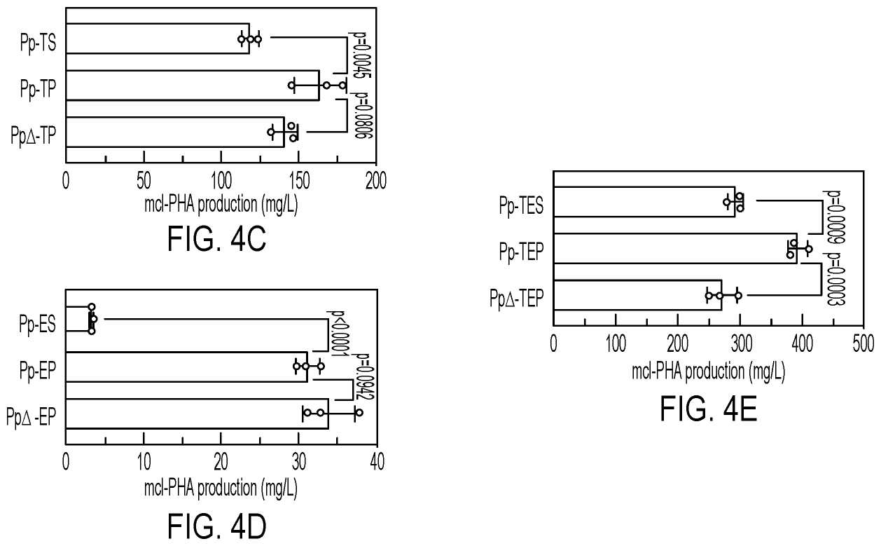

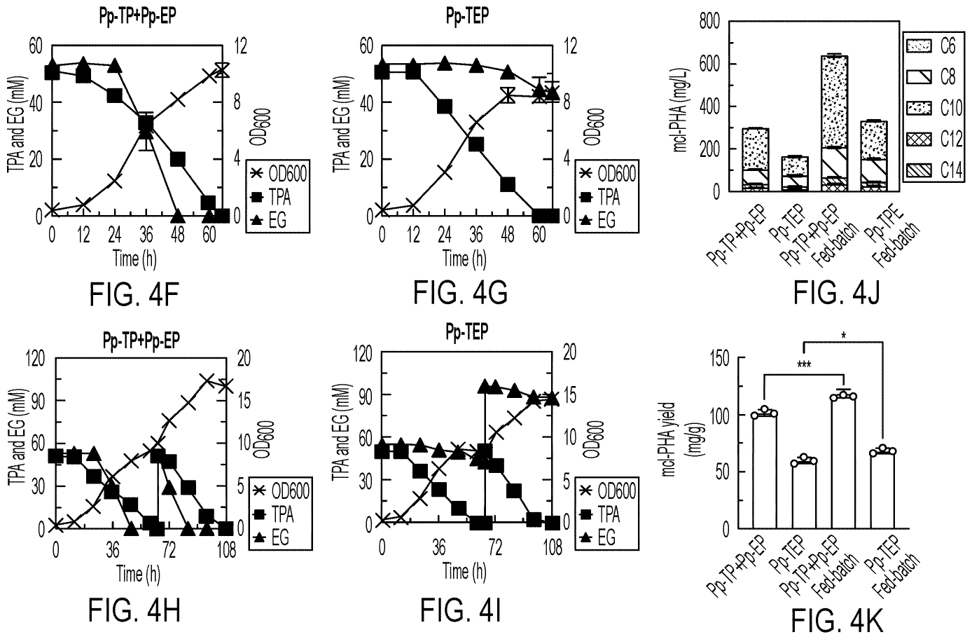

To achieve the goal, two parallel strategies were applied (FIG. 4A). First, we overexpressed the genes phaG and alkK, which encode hydroxyacyl-ACP acyltransferase and acyl-CoA-synthase, respectively, to catalyze the conversion of (R)-3-hydroxyacyl-ACP to (R)-3-hydroxyacyl-CoA. The two enzymes are also essential for PHA biosynthesis under high nitrogen conditions and cell growth on aromatic compounds. Therefore, we used a constitutive promoter Pₑₘ₇ to tandemly drive phaG and alkK, linked with two mcl-PHA synthases (phaC1 and phaC2), in both Pp-T and Pp-E, resulting in the derivatives Pp-TP and Pp-EP, respectively. Second, we deleted from Pp-TP and Pp-EP FadBA and FadBAxE to limit the carbon flux from (R)-3-hydroxyacyl-CoA to acetyl-CoA, as well as the PHA depolymerase (phaZ) to eliminate PHA depolymerization, yielding new derivatives PpΔ-TP and PpΔ-EP, respectively. Similarly, we engineered the co-degradation strain Pp-TE (FIG. 4B), resulting in Pp-TEP and PpΔ-TEP accordingly. For comparison, we additionally constructed three control strains-Pp-TS, Pp-ES and Pp-TES—by transforming the vector pSEVA421 into the corresponding parental strains, Pp-T, Pp-E and Pp-TE.

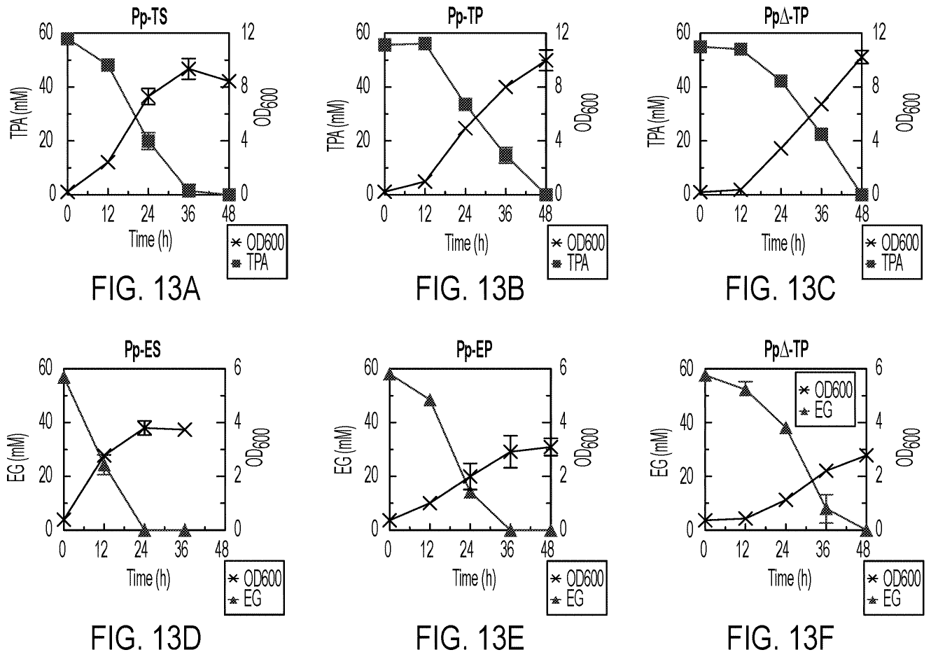

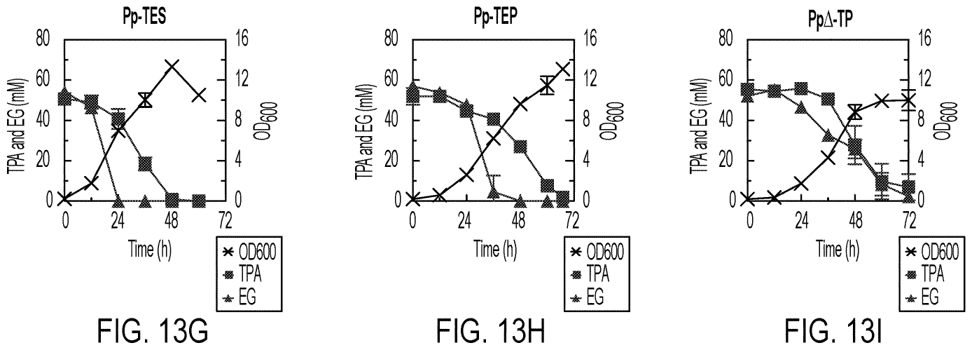

To evaluate these strains, we performed monoculture fermentations using M9 medium supplemented with pure TPA and/or EG. During the TPA fermentation, Pp-TP and PpΔ-TP both produced more mcl-PHA (163.78±16.81 and 140.96±8.04 mg/L, respectively) than their control Pp-TS (118.25±5.37 mg/L) (FIG. 4C). However, they were 1.25 and 2.38 times slower in TPA depletion than Pp-TS (FIG. 13A-13C), likely owing to the genetic manipulations we implemented. Similarly, Pp-EP and PpΔ-EP exhibited a significant increase in mcl-PHA production (31.25±1.57 and 34.82±2.73 mg/L) compared with Pp-ES (3.39±0.23 mg/L) during pure EG fermentation (FIG. 4d) but, accordingly, had an associated delay in EG depletion (FIG. 13D-13F). Notably, the absolute mcl-PHA yields of Pp-EP and PpΔ-EP were much lower than those of Pp-TP and PpΔ-TP, owing to a lower carbon content in EG than in TPA and hence a lower carbon to nitrogen ratio (C:N ratio) in growth medium, a critical regulator for mcl-PHA accumulation. For single-strain co-utilization of pure TPA and EG mixture, Pp-TEP exhibited a higher production (392.64±15.70 mg/L) than the control Pp-TES (292.89±12.37 mg/L), but PpΔ-TEP was lower in mcl-PHA titer (271.58±23.74 mg/L) (FIG. 4E), even though the substrate degradation rates of Pp-TEP and PpΔ-TEP all reduced (FIG. 13G-13I). These experiments confirmed the phenotypes of our engineered strains, and also revealed Pp-TP, Pp-EP and Pp-TEP as the best upcycling candidates.

Next, we used the co-culture of Pp-TP and Pp-EP, named the TP-EP consortium, and the single-strain counterpart Pp-TEP to ferment PET hydrolysate for mcl-PHA production under nitrogen-limiting conditions. FIG. 4F shows that the consortium simultaneously consumed TPA and EG at comparable rates, leading to the depletion of the hydrolysate in about 65 h. By contrast, Pp-TEP utilized all TPA but was significantly slower in EG degradation, resulting in significant EG remanent (82%) (FIG. 4G). Here, compared with the pure mixture case (FIG. 13H), Pp-TEP's EG degradation capacity was greatly impaired during the hydrolysate fermentation. Associated with its advantage in substrate utilization, the TP-EP consortium also showed a higher mcl-PHA productivity (296.46±4.70 mg/L) than Pp-TEP (164.22 292.89±8.27 mg/L) (FIG. 4J). Additionally, to evaluate the stability and performance of the engineered systems, we performed fed-batch fermentation with pulse feeding. The TP-EP consortium continued to successfully deplete both TPA and EG at the hydrolysate feeding stage (FIG. 4H). By contrast, limited EG uptake of Pp-TEP led to an EG accumulation upon feeding, which reached 88.45 mM at the end of fermentation (FIG. 4I). Subsequent analysis showed that the fed-batch of the TP-EP consortium and Pp-TEP both yielded significantly more mcl-PHA than the single batch fermentation (FIG. 4J). Remarkably, through the fed-batch fermentation, the consortium yielded 637.30±10.14 mg/L mcl-PHA, which was about 92% higher than that of Pp-TE. A detailed monomer composition analysis of the product revealed that, although the longer chain monomers such as 3-hydroxydodecanoate (C12) and 3-hydroxytetradecanoate (C14) exhibited the largest increases, the majority of the product was 3-hydroxydecanoate (C10) and 3-hydroxyoctanoate (C8) which accounted for >75% of the total. Thus, the consortium design presented two advantages over its single-strain counterpart for bioconversion—a faster and complete deconstruction of the hydrolysate and a higher production titer for mcl-PHA—showcasing the potential of engineered consortia for polymer upcycling.

### Example 5 High-Yield MA Biosynthesis Via DOL-Mediated System Optimization

Microbial DOL offers unique traits such as functional specialization and crosstalk reduction as demonstrated above for engineering cellular functions. In principle, it also promises additional benefits for bioproduction by alleviating biomolecular toxicities and facilitating systematic optimization. To illustrate this point, we set out to upcycle the PET hydrolysate into cis-cis MA, an important chemical used to synthesize polyurethane and the nylon precursor adipic acid. Although MA biosynthesis has been demonstrated, engineered strains were limited to glucose feeding and, additionally, suffers from the accumulation of intermediate metabolites. One such intermediate is catechol (CAT), which can be converted from TPA and bioprocessed into MA. As CAT is toxic to microbes, its accumulation may lead to forced self-toxification, which impairs MA production. Thus, we designed a new ecosystem, termed TC-EM consortium, by introducing an additional layer of DOL into the T-E consortium to enable MA biosynthesis by using the PET hydrolysate as the sole carbon source without additional carbon supply.

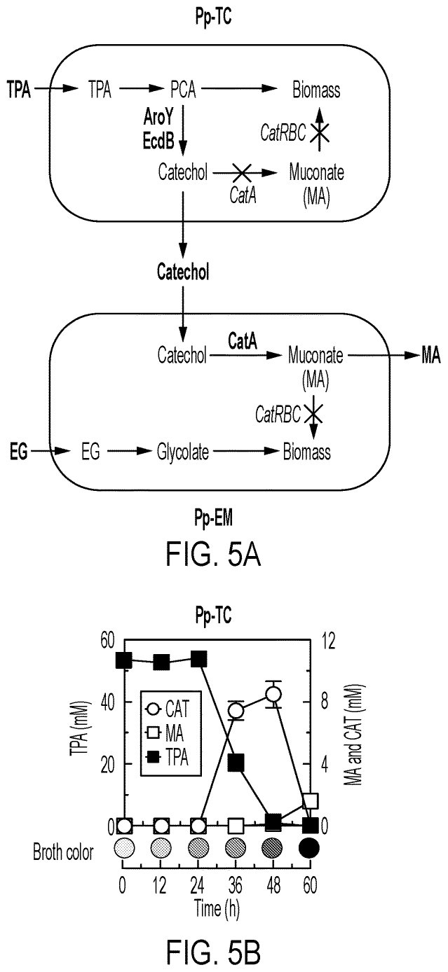

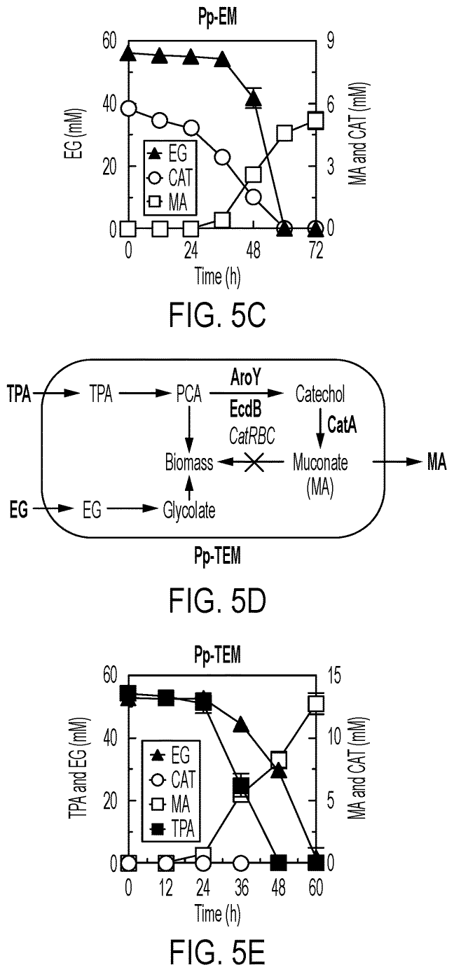

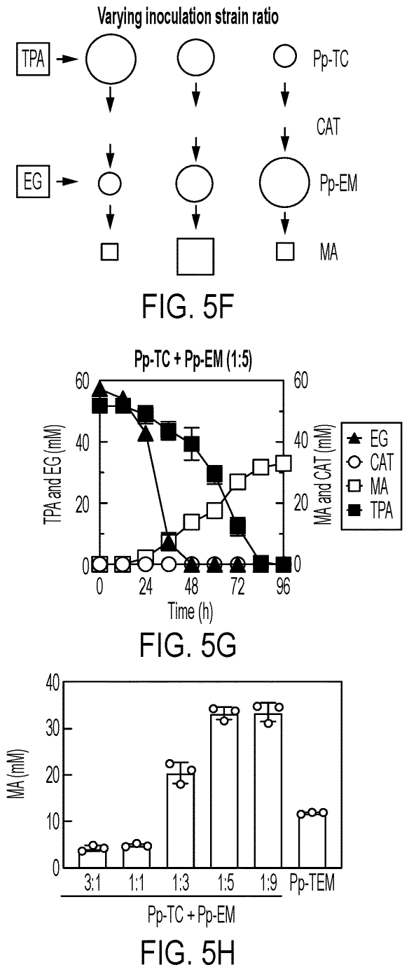

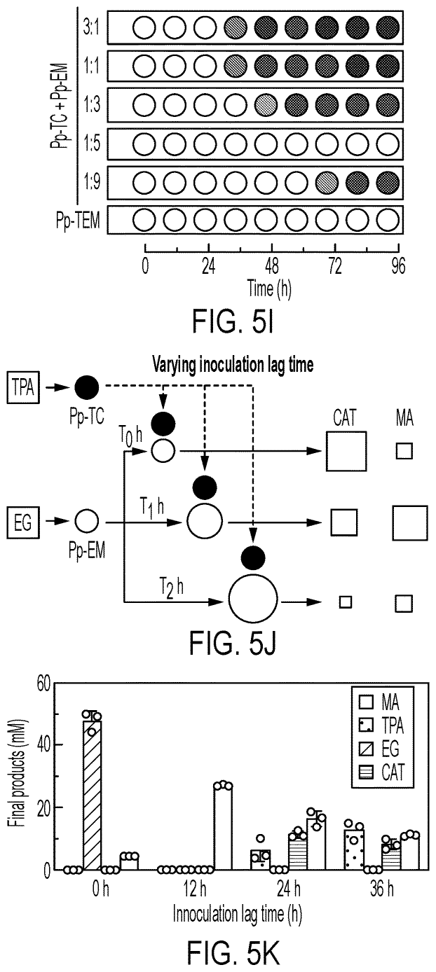

As illustrated in FIG. 5A, the MA synthesis pathway was split into an upstream subsystem that converts TPA to CAT and a downstream subsystem upgrading CAT into MA. These two subsystems were then loaded into Pp-T and Pp-E, respectively, to generate Pp-TC and Pp-EM which constitute the TC-EM consortium. Specifically, Pp-TC was created by blocking the main CAT catabolic pathway by deleting the catRBCA cluster and introducing a codon-optimized PCA decarboxylase (aroY) and decarboxylase enhancer (ecdB) from Enterobacter cloacae ATCC 13047. Pp-EM was constructed by deleting the catRBC operon and introducing the tac promoter (P_{tac}) to substitute the native promoter of the major catechol 1,2-dioxygenase (catA)⁴ to boost the CAT-to-MA conversion.

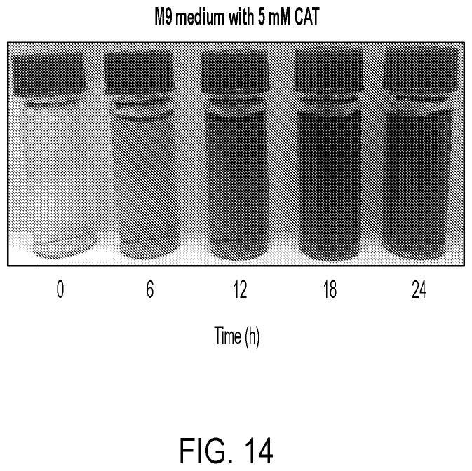

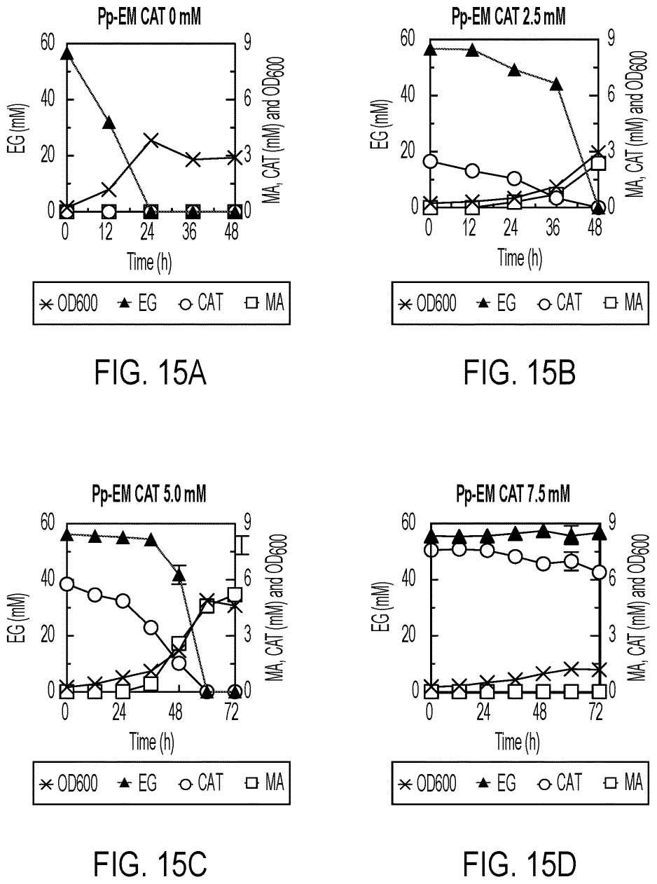

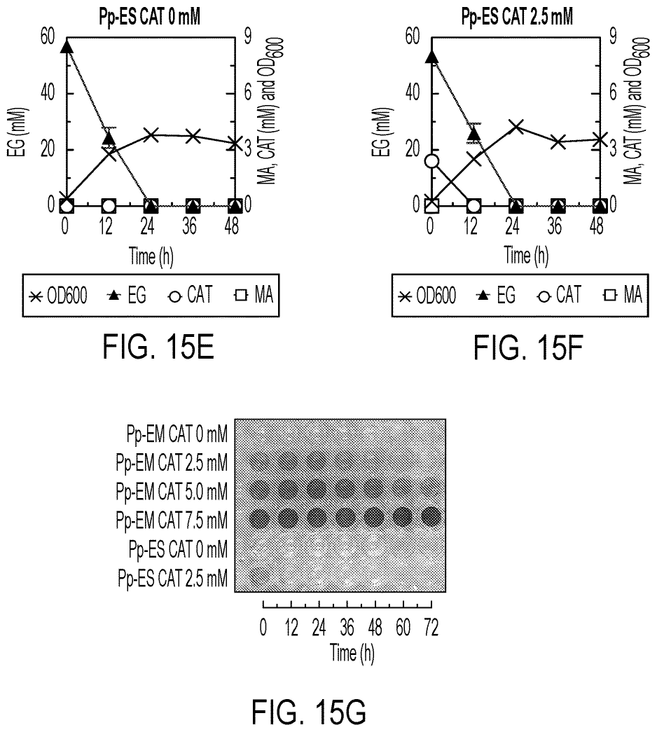

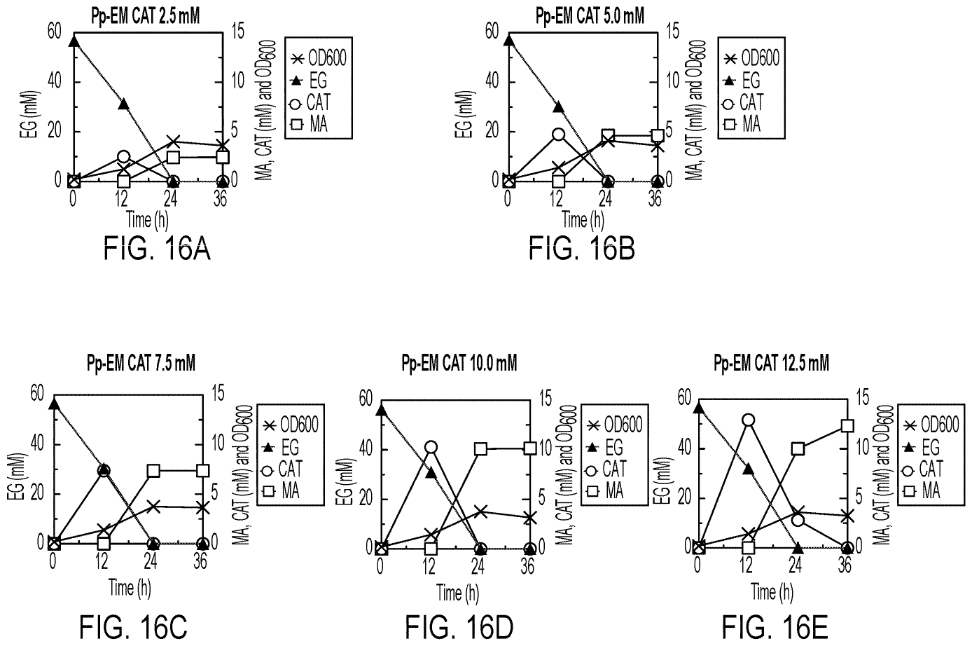

Fermentations showed that Pp-TC successfully degraded 53.41 mM TPA over a course of 48 h during which CAT accumulated up to 8.48±0.86 mM; afterwards, CAT declined sharply along with a minor MA generation, resulting in CAT depletion and MA accumulation (1.60±0.19 mM) at 60 h (FIG. 5B). Here, MA was synthesized because Pp-TC contains the secondary catechol 1,2-dioxygenase (catA2), which plays as a safety valve to convert excess CAT to MA. Additionally, the dynamic CAT fluctuation was accompanied by the change of culture color from transparent to brown and black, which was caused by the mixing and reaction of CAT and Fe³⁺ that yields “brown carbon” (FIG. 14). Pp-EM showed a rapid EG consumption without any MA production in the absence of CAT (FIG. 15A). When a low to intermediate level (2.48-5.77 mM) of CAT was supplemented at the beginning of the experiment, Pp-EM not only fully degraded EG but also converted CAT to MA (FIG. 5C and FIG. 15B, 15C). For example, Pp-EM depleted 56.23 mM EG and yielded 5.20±0.42 mM MA from 5.77 mM CAT in 60 h, reaching nearly 90% of CAT conversion (FIG. 5D). By contrast, the control Pp-ES did not yield MA with or without CAT (FIG. 15E, 15F), confirming that MA was synthesized primarily through the engineered pathway. Importantly, there was an increasing delay in fermentation with the increase in CAT. Notably, Pp-EM was fully inhibited in the presence of 7.59 mM CAT (FIG. 15D). Surprisingly, when CAT was supplemented after Pp-EM entered its exponential growth phase (e.g., after 12 h), it affected neither cell growth nor EG degradation, and was fully converted to MA with a 100% yield (FIG. 16), suggesting that CAT strongly impairs initial cell growth but not the catalysis underlying the bioconversion.

For comparison, we also devised a control strain, Pp-TEM, that consumes both TPA and EG for MA production (FIG. 5D). The strain was created by transforming into Pp-EM the TPA degradation and CAT biosynthesis plasmids used in Pp-TC. Confirming the design, a mixed TPA and EG fermentation (56 mM each) by Pp-TEM resulted in complete substrate utilization and the synthesis of 12.74±0.85 mM MA (FIG. 5E).

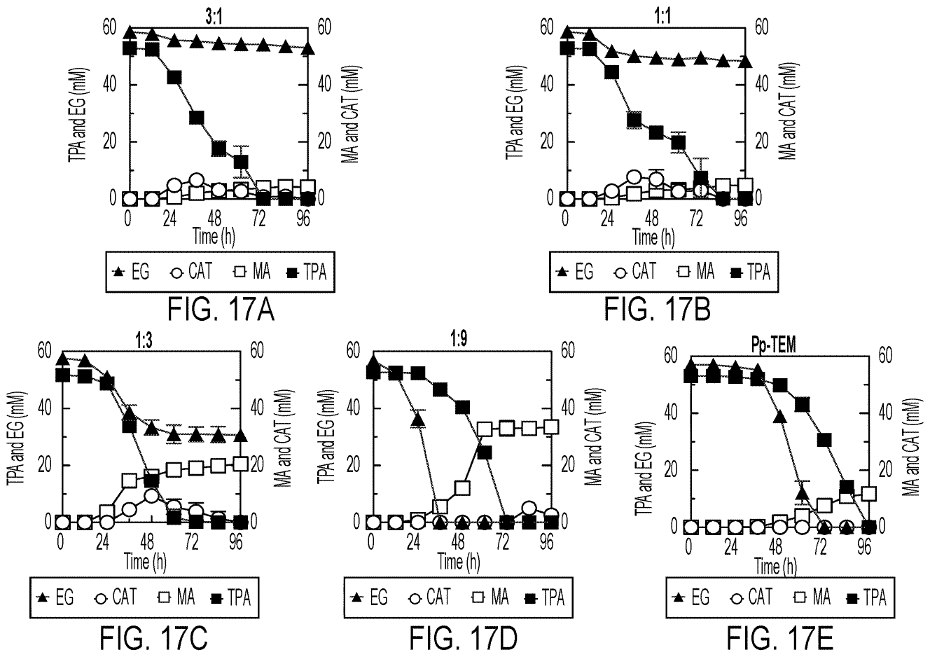

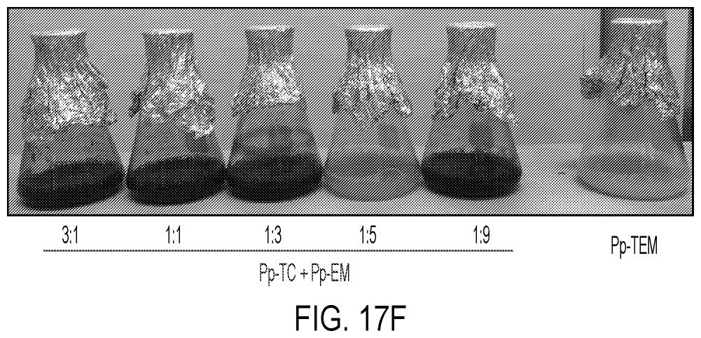

Compared with Pp-TEM, the strains Pp-TC and Pp-EM each have a reduced burden from TPA and EG catabolismmA synthesis, CAT detoxification and stress response. Additionally, by separating the MA pathway at the CAT node, the TC-EM consortium possesses a unique degree of freedom for production optimization. Specifically, by varying the initial Pp-TC:Pp-EM ratio, the CAT influx from TPA and outflux to MA can be altered, thereby enabling one to balance metabolic fluxes and minimize CAT accumulation (FIG. 5F). Indeed, hydrolyzed PET fermentations with different inoculation ratios yielded varied temporal patterns and final titers (FIG. 5G, 5F and FIG. 17). With a 3:1, 1:1 or 1:3 ratio, the CAT synthesis by Pp-TC was particularly strong but the CAT-to-MA conversion by Pp-EM was weak, leading to CAT accumulation (FIG. 17A-17C). Consequently, EG degradation was abolished and MA production was inefficient, accompanied by “brown carbon” accumulation as illustrated by the blackening of the culture (FIG. 5I and FIG. 17F). At the optimal ratio of 1:5, TPA and EG were both fully consumed without CAT accumulation or culture blackening (FIG. 5G, 5I and FIG. 17F), with a total of 31.89±2.20 mM final MA that was 2.99-fold higher than that from the Pp-TEM fermentation (FIG. 5H and FIG. 17E). At the 1:9 ratio, the substrates were depleted and the MA yield remained high; however, there was again the occurrence of CAT accumulation and blackening of the culture (FIG. 17D, 17F and FIG. 5I). These results demonstrated initial ratio alteration as an effective strategy to optimize the MA biosynthesis.

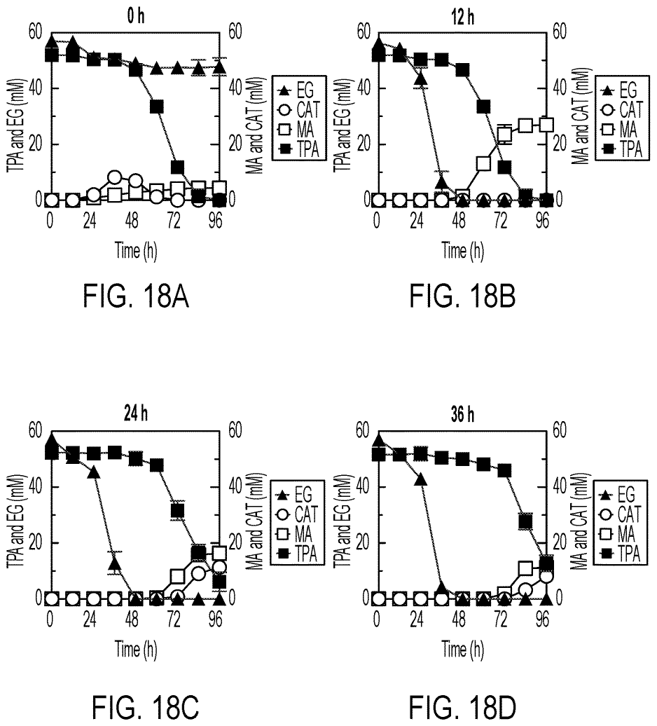

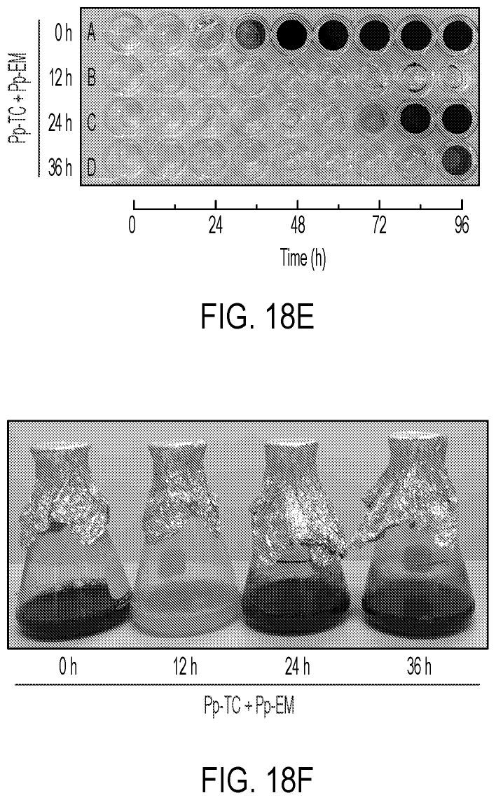

In principle, MA production can be additionally modulated by varying the time lag between Pp-TC and Pp-EM inoculations, owing to the facts that efficient MA production requires rapid CAT-to-MA conversion to minimize the CAT accumulation and pre-inoculating Pp-EM enhances the catalysis of the bioprocessing (FIG. 5J). We thus performed another set of hydrolysate fermentations, with Pp-EM being inoculated at the beginning but Pp-TC supplemented later at different time points. Among four conditions tested (FIG. 18A-18D), we found that a delay of 12 h gave rise to the optimal performance with the completed TPA and EG consumption, no CAT accumulation and highest MA production (FIG. 5K and FIG. 18B). By contrast, no or excessive delays led to incomplete or delayed substrate utilization, CAT accumulation and suboptimal MA production (FIG. 18A, 18C, 18D). Consistent with these results, the colors of the co-cultures changed accordingly throughout the fermentations (FIG. 18E, 18F). Collectively, these experiments demonstrated that, in addition to realizing MA production with PET hydrolysate, microbial DOL provides a powerful tunability for simultaneous optimization of product synthesis and substrate deconstruction.

### Example 6 Simultaneous mcl-PHA and Itaconic Acid Production

To further showcase the utility of engineered consortia, we assessed the feasibility of producing multiple products from hydrolyzed PET in a single fermentation. Specifically, we designed a pair of strains, namely Pp-TAIP and Pp-EAIP, that specialize in assimilating TPA and EG respectively and utilizing the substrates to co-produce mcl-PHA and itaconic acid (IA), a building block chemical used for resins, paints, synthetic fibers, and others. We chose these two chemicals for a proof-of-concept demonstration because they are accumulated in different compartments, insoluble mcl-PHA granules in the bacterial cytoplasm and soluble IA in the fermentation broth, making it simple for downstream product separation and recovery.

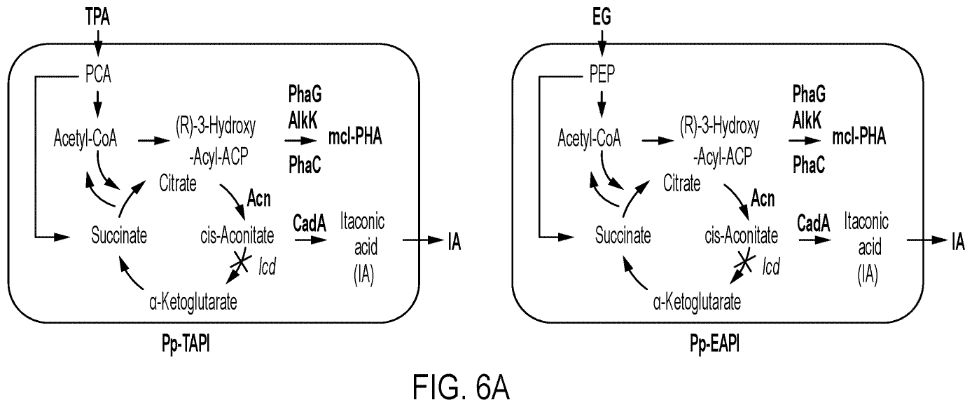

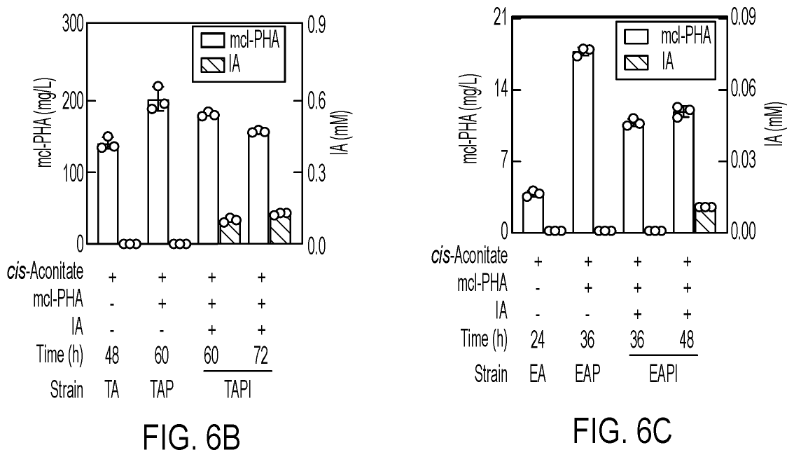

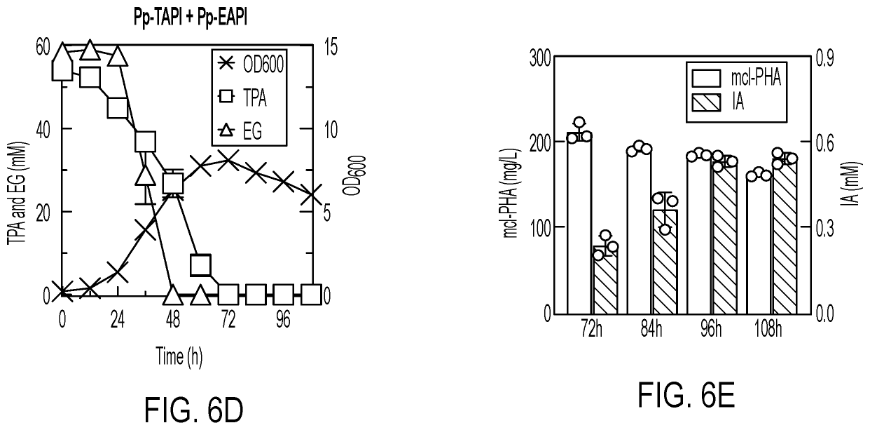

As shown in FIG. 6A, building from the TPA- and EG-utilizing specialists Pp-T and Pp-E, we first increased their carbon flux toward cis-aconitate, the precursor of IA, by weakening the start codon of isocitrate dehydrogenase (icd) from ATG to GTG and introducing the heterologous aconitate hydratase gene (acn) from Corynebacterium glutamicum strain ATCC 13032 at the downstream of icd in the genome, which resulted in the derivatives Pp-TA and Pp-EA accordingly. Subsequently, we boosted mcl-PHA production by using the constitutive promoter, Pₑₘ₇, to drive phaG, alkK, phaC1, and phaC2 as in the case of Pp-TP and Pp-EP, resulting in the strains Pp-TAP and Pp-EAP. Finally, we utilized an additional Pₑₘ₇ promoter to drive the cis-aconitic acid decarboxylase gene (cadA) from Aspergillus terreus to confer IA production, which generated the final strains Pp-TAPI and Pp-EAPI.

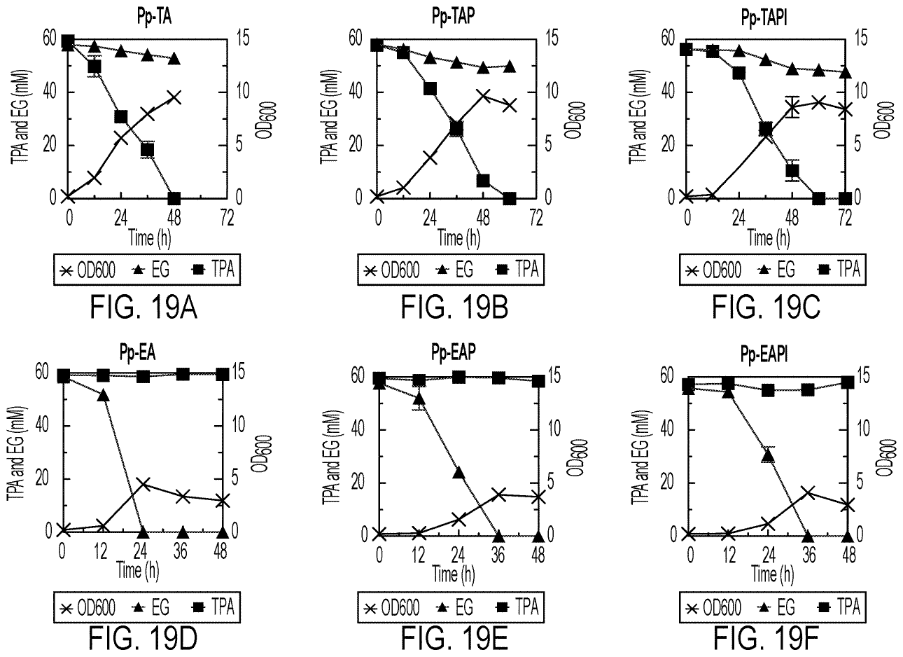

Next, we evaluated these strains in monoculture fermentations using pure TPA and EG (56 mM each) mix as the substrates. The three Pp-T derivative strains (Pp-TA, Pp-TAP, and Pp-TAPI) gave rise to a similar substrate utilization pattern, i.e., rapid and successful TPA consumption and a minimal EG degradation (FIG. 19A-19C). However, they had distinct outcomes in terms of product titers (FIG. 6B). Pp-TA produced mcl-PHA but not IA, as the organism has an innate ability of PHA production but is incomplete in IA synthesis even though the flux to cis-aconitate was enhanced. Pp-TAP yielded a higher level of mcl-PHA than Pp-TA, due to the introduction of heterologous enzyme genes for PHA synthesis; however, it remained to be deficient in IA production. By contrast, Pp-TAPI produced both mcl-PHA and IA owing to the completeness of the introduced pathways. Likewise, we observed a comparable substrate assimilation characteristic for the three Pp-E derivatives (Pp-EA, Pp-EAP, and Pp-EAPI) whereby EG was rapidly consumed while TPA remained intact (FIG. 19D-19F). Additionally, these strains showed product profiles similar to those of the Pp-T derivatives, although the product titers were much lower because of the lower carbon content in EG than in the same mole of TPA (FIG. 6C). These experiments confirmed our successful pathway engineering in the designed strains.

To validate mcl-PHA and IA co-production, we utilized the co-culture of Pp-TAPI and Pp-EAPI (the TAPI-EAPI consortium) to ferment the PET hydrolysate. We found that, when inoculated with the 1:3 ratio, the consortium completely degraded both TPA and EG in the hydrolysate in less than 72 h and yielded cellular biomass accordingly (FIG. 6D). Through the course, the co-culture successfully produced both mcl-PHA and IA (FIG. 6E). Specifically, it produced 210.94±10.18 mg/L of mcl-PHA at 72 h at which the hydrolysate was depleted, followed by a slow decline to 161.23±2.62 mg/L at 108 h. Different from mcl-PHA which reached its maximum at 72 h, the majority of IA was produced during the stationary phase and reached the maximum of 0.53±0.02 mM at 96 h. We also noticed the PHA titer was lower than that in FIG. 4J, which is because the growth setting for the PHA and IA co-production here was different from the nitrogen-limiting condition optimized for sole mcl-PHA production.

### Example 8 Conclusions

Through the engineering of microbial DOL, we established a synthetic biology platform that efficiently degrades PET hydrolysate and subsequently generates desired products of interest. Compared with its monoculture counterpart, in our experiments the consortium was more effective in deconstructing TPA and EG, particularly when their concentrations were high or crude hydrolysate was directly used. It also surpassed single strains in terms of upcycling performance for both mcl-PHA and MA biosynthesis, by conferring flexible system optimization and reducing catabolic crosstalk and chemical toxicity. Additionally, it was demonstrated for the simultaneous production of multiple value-added products (mcl-PHA and IA).

### Example 8 Methods

Strains, plasmids, and culture condition. All bacterial strains and plasmids used or constructed in this study are listed in Table 1.

TABLE 1
Strains and plasmids used in this study.

| | | Source or |
|---|---|---|
| Name | Description | Reference |
| Strains | | |
| Pseudomonas | KT2440 derivative; Δprophage1 Δprophage4 Δprophage3 | Martinez- |
| putida EM42 | Δprophage2 ΔTn7 ΔendA-1 ΔendA-2 ΔhsdRMS Δflagellum | Garcia et |
| | ΔTn4652 | al., 2014 |
| Pp00 (Pp-T₀) | EM42 derivative; contains the plasmid pBb(B5)1k-Ptac- | This study |
| | tpa, Km^{R} | |
| Pp01 | EM42 derivative; Δped | This study |
| Pp01A | Pp01 derivative; icd^{GTG::ATG} with synthetic expression | This study |
| | cassette (ptac-acn) in chromosome | |
| Pp02 (Pp-T) | Pp01 derivative; contains the plasmid pBb(B5)1k-Ptac-tpa, | This study |
| | Km^{R} | |
| Pp03 (Pp-TS) | Pp01 derivative; contains the plasmids pBb(B5)1k-Ptac- | This study |
| | tpa and pSEVA421, Sm^{R}, km^{R} | |
| Pp04 (Pp-TP) | Pp01 derivative; contains the plasmids pBb(B5)1k-Ptac- | This study |
| | tpa and pSEVA421-phaG-alkK-phaC1-phaC2, Sm^{R}, km^{R} | |
| Pp05 | Pp01 derivative; Δped, ΔphaZ, ΔfadBA ΔfadBAxE | This study |
| Pp06 (PpΔ-TP) | Pp05 derivative; Contains the plasmids pBb(B5)1k-Ptac- | This study |
| | tpa and pSEVA421-phaG-alkK-phaC1-phaC2, Sm^{R}, km^{R} | |
| Pp07 | Pp01 derivative; ΔcatRBCA | This study |
| Pp08 (Pp-TC) | Pp07 derivative; Contains the plasmids pBb(B5)1k-Ptac- | This study |
| | tpa and pSEVA421-aroY-EcdB, Sm^{R}, km^{R} | |
| Pp09 (Pp-TA) | Pp01A derivative; contains the plasmid pBb(B5)1k-Ptac- | This study |
| | tpa, Km^{R} | |
| Pp010 (Pp-TAP) | Pp01A derivative; contains the plasmid pBb(B5)1k-Ptac- | This study |
| | tpa, and pSEVA421-phaG-alkK-phaC1-phaC2, Sm^{R}, km^{R} | |
| Pp011 (Pp-TAPI) | Pp01A derivative; contains the plasmid pBb(B5)1k-Ptac- | This study |
| | tpa, and pSEVA421-cadA-phaG-alkK-phaC1-phaC2, Sm^{R}, | |
| | km^{R} | |
| Pseudomonas | EM42 derivative; UV mutation, has an ability to | Lab stock |
| putida M31 | metabolize ethylene glycol | |
| PpM01 | M31 derivative; ΔgclR | This study |
| PpM02 (Pp-E) | PpM02 derivative; synthetic expression cassette (ptac- | This study |
| | gclDEF) in chromosome | |
| PpM03 (Pp-TE) | PpM02 derivative; Contains the plasmid Contains the | This study |
| | plasmid pBb(B5)1k-Ptac-tpa, Km^{R} | |
| PpM03 (Pp-ES) | PpM02 derivative; Contains the plasmid pSEVA421, Sm^{R} | This study |
| PpM04 (Pp-EP) | PpM02 derivative; Contains the plasmid pSEVA421-phaG- | This study |
| | alkK-phaC1-phaC2, Sm^{R} | |
| PpM05 (Pp-TES) | PpM02 derivative; Contains the plasmids pBb(B5)1k-Ptac- | This study |
| | tpa and pSEVA421, Sm^{R}, Km^{R} | |
| PpM06 (Pp-TEP) | PpM02 derivative; Contains the plasmids pBb(B5)1k-Ptac- | This study |
| | tpa and pSEVA421-phaG-alkK-phaC1-phaC2, Sm^{R}, Km^{R} | |
| PpM06 (PpΔ-E) | PpM02 derivative; Δped ΔphaZ ΔfadBA ΔfadBAxE | This study |
| PpM07 (PpΔ-EP) | PpM06 derivative; Contains the plasmid pSEVA421-phaG- | This study |
| | alkk-phaC1-phaC2, Sm^{R} | |
| PpM08 (PpΔ-TEP) | PpM06 derivative; Contains the plasmids pBb(B5)1k-Ptac- | This study |
| | tpa and pSEVA421-phaG-alkK-phaC1-phaC2, SmR, Km^{R} | |
| PpM09 | PpM02 derivative; ΔcatRBC with synthetic expression | This study |
| | cassette (ptac-catA) in chromosome | |
| PpM10 (Pp-EM) | PpM09 derivative; Contain the plasmid pSEVA421, Sm^{R}, | This study |
| PpM11 (Pp-TEM) | PpM09 derivative; Contains the plasmids pBb(B5)1k-Ptac- | This study |
| | tpa and pSEVA421-aroY-ecdB, Sm^{R}, km^{R} | |
| PpM12 (Pp-EA) | PpM02 derivative; icd^{GTG::ATG} with synthetic expression | This study |
| | cassette (ptac-acn) in chromosome | |
| PpM13 (Pp-EAP) | PpM12 derivative; Contains the plasmid pSEVA421-phaG- | This study |
| | alkK-phaC1-phaC2, Sm^{R} | |
| PpM14 (Pp-EAPI) | PpM12 derivative; Contains the plasmid pSEVA421-cadA- | This study |
| | phaG-alkK-phaC1-phaC2, Sm^{R} | |
| Plasmids | | |
| pBb(B5)1k-GFPuv | E. coli and P. putida shuttle vector, IPTG-inducible P_{trc} | Taylor B. |
| | promoter expressing gfpuv, BBR1-B5 origin, km^{R} | Cook1, et |
| | | al., 2018 |
| pBb(B5)1k-tpa | Expression plasmid, Constitutive expression of tpa operon | This study |
| | (tpaAa-tpaAb-tpaC-tpB-tpak from Rhodococcus jostii | |
| | RHA1) using P_{tac} promoter, km^{R} | |
| pSEVA421 | E. coli and P. putida shuttle vector, oriV (RK2) origin; Sm^{R} | Silva- |
| | | Rocha et |
| | | al., 2012 |
| pSEVA421-phaG- | Expression plasmid, Constitutive expression of phaG-alkk- | This study |
| alkK-phaC1-phaC2 | phaC1-phaC2 using EM7 promoter, Sm^{R} | |
| pSEVA421-aroY- | Expression plasmid, Constitutive expression of aroY-ecdB | This study |
| ecdB | using Ptac promoter, Sm^{R} | |
| pSEVA421-cadA | Expression plasmid, Constitutive expression of cadA using | Lab stock |
| | EM7 promoter, Sm^{R} | |
| pSEVA421-cadA- | Expression plasmid, Constitutive expression of cadA and | This study |
| phaG-alkK-phaC1- | phaG-alkK -phaC1-phaC2 using EM7 promoters, Sm^{R} | |
| phaC2 | | |
| pK18mobsacB | Gene knockout vector, Kan^{R} | Schafer et |
| | | al., 1994 |
| pK18-ped | pK18mob sacB containing the homologous arms of the | This study |
| | ped DNA region of EM42, Kan^{R} | |
| pK18-gclR | pK18mob sacB containing the homologous arms of the | This study |
| | gcIR DNA region of EM42, Kan^{R} | |
| pK18- Pro-glcDEF | Gene knockout vector, pK18mob sacB containing the | This study |
| | homologous arms of the gclR DNA region of EM42 and | |
| | ptac-glcDEF cassette, Kan^{R} | |
| pK18-phaZ | pK18mob sacB containing the homologous arms of the | This study |
| | phaZ DNA region of EM42, Kan^{R} | |
| pK18-fadBA | pK18mob sacB containing the homologous arms of the | This study |
| | fadBA DNA region of EM42, Kan^{R} | |
| pK18-fadBAxE | pK18mob sacB containing the homologous arms of the | This study |
| | fadBAxE DNA region of EM42, Kan^{R} | |
| pK18-catRBC | pK18mob sacB containing the homologous arms of the | This study |
| | catRBC DNA region of EM42, Kan^{R} | |
| pK18-catRBCA | pk18mob sacB containing the homologous arms of the | This study |
| | catRBCA DNA region of EM42, Kan^{R} | |
| pK18-icdGTG-acn | pK18mob sacB containing the homologous arms of the icd | This study |
| | DNA region of EM42 and ptac-acn cassette, Kan^{R} | |

Primers used in this work for DNA fragment amplification or verification are listed in Table 2.

TABLE 2
Sequence information for genes, promoters and insertion
fragments.

| | | |
|---|---|---|
| Primer Name | Primer sequence | Description |

For ped gene deletion plasmid construction

| | | |
|---|---|---|
| PK18-F | CCGGATGAATGTCAGCTACT (SEQ ID NO: 1) | To clone the 5100 bp DNA |
| PK18-R | GCGGTAATACGGTTATCCAC (SEQ ID NO: 2) | fragment from the |
| | | pK18mobsacB |
| ped-UF | TACAGGCCCCACCGCGTCGCGGCCTTC (SEQ ID NO: 3) | To clone the 1481 bp |
| ped-UR | AGTAGCTGACATTCATCCGGTCTCGCGCAGCGCAATATCAG | upstream DNA fragment of |
| | TAGGG (SEQ ID NO: 4) | ped gene from the P. putida |
| | | EM42 chromosome |
| ped-DF | GTGGATAACCGTATTACCGCGCTGGTGCACAACTTGTATCC | To clone the 1292 bp |
| | G (SEQ ID NO: 5) | downstream DNA fragment |
| ped-DR | GACGCGGTGGGGCCTGTAGGCGC (SEQ ID NO: 6) | of ped gene from the P. |
| | | putida EM42 chromosome |
| V-ped-F | TGCGGGTGGCGGGGTGA (SEQ ID NO: 7) | To verify the ped gene |
| V-ped-R | TTAATGCACGGGGGCGGACTG (SEQ ID NO: 8) | deletion strain, wild strain |
| | | yields 10002 bp, the Aped |
| | | yields 409 bp. |

For gclR gene deletion plasmid construction

| | | |
|---|---|---|
| pk18-F1 | TGGCGCACATCCATAAGCTTGGCACTGGCCGTCGTTTTACA | To clone the 5724 bp DNA |
| | A (SEQ ID NO: 9) | fragment from the |
| pk18-R1 | CAGATAACCCTTCTGGGATCCCCGGGTACCGAGCTCGAATT | pK18mobsacB |
| | CGTAA (SEQ ID NO: 10) | |
| gclR-UF | CACGCAGGCAAACGAGGGCTCTCGAGGCACGAAGAGAA | To clone the 1462 bp |
| | AGTATGG (SEQ ID NO: 11) | upstream DNA fragment of |
| gclR-UR | GCCAGTGCCAAGCTTATGGATGTGCGCCATGCTGCCCATCA | gcIR gene from the P. putida |
| | TC (SEQ ID NO: 12) | EM42 chromosome |
| gclR-DF | GGTACCCGGGGATCCCAGAAGGGTTATCTGGACGTCTACG | To clone the 1431 bp |
| | ATCACTTCGAGCTGC (SEQ ID NO: 13) | downstream DNA fragment |
| gclR-DR | GTGCCTCGAGAGCCCTCGTTTGCCTGCGTGATCGAGGGGC | of gclR gene from the P. |
| | TG (SEQ ID NO: 14) | putida EM42 chromosome |
| V-gclR-F | ATCACCTGGGCGATCACATAAGGCAG (SEQ ID NO: 15) | To verify the ped gene |
| V-gclR-R | CCATGATGTTGGGGTTGACGAAGATGATGTAGG (SEQ ID | deletion strain, wild strain |
| | NO: 16) | yields 1646 bp, the AgclR |
| | | yields 409 bp. |

For Ptac-gclDEF integration plasmid construction

| | | |
|---|---|---|
| PK18-F | CCGGATGAATGTCAGCTACT (SEQ ID NO: 17) | To clone the 5100 bp DNA |
| PK18-R | GCGGTAATACGGTTATCCAC (SEQ ID NO: 18) | fragment from the |
| | | pK18mobsacB |
| glcDEF-UF | ATGAATATCCTGTACGACGAACGCGTCG (SEQ ID NO: 19) | To clone the 1520 bp |
| glcDEF-UR | AGTAGCTGACATTCATCCGGTCAGAAGCGCTCCAGCTCGG | upstream DNA fragment of |
| | GGAAGG (SEQ ID NO: 20) | glcDEF operon from the P. |
| | | putida EM42 chromosome |
| gclDEF-DF | GTGGATAACCGTATTACCGCCCGCTTCGCTGTATTCGACCT | To clone the 1254 bp |
| | TCAAGG (SEQ ID NO: 21) | downstream DNA fragment |
| glcDEF-DR | CGGCTCACTCGCAACGGTTTTTGTTGTTGTTGG (SEQ ID | of glcDEF operon from the P. |
| | NO: 22) | putida EM42 chromosome |
| | | 1254 bp |
| V-glcDEF-F | ATATCGCCAATCTAGCCAGACAGAACCG (SEQ ID NO: 23) | To verify the constitutively |
| V-glcDEF-R | TGAACAATGCCAGCAGGTCGAAGC (SEQ ID NO: 24) | expression glcDEF strain, wild |
| | | strain yields 790 bp, the |
| | | engineered strain yields 897 |
| | | bp |
| Ptac-RBS | ACAACAACAAAAACCGTTGCGAGTGAGCCGCGCAAAAAA | Synthesized DNA fragment of |
| | CCGCACCCAGGTGCGGTTTTTTGAATTCGAGCTGTTGACA | strong constitutive promoter |
| | ATTAATCATCGGCTCGTATAATGTGTCAGACTCAATAATAATA | (Ptac) and an artificial RBS. |
| | ATAAGGAGGTATCGAATGAATATCCTGTACGACGAACGCGT | Gibson overlaps are |
| | CG (SEQ ID NO: 25) | underlined. |

For catRBC gene deletion plasmid construction

| | | |
|---|---|---|
| catRBC-UF | ACCTGACCTCCATAGACACACCATGCCCACAGGGG (SEQ ID | To clone the 1234 bp |
| | NO: 26) | upstream DNA fragment of |
| catRBC-UR | GTAGCTGACATTCATCCGGCAAGGCGCAGGAAAAAGGGT | catRBC gene from the P. |
| | TGC (SEQ ID NO: 27) | putida EM42 chromosome |
| catRBC-ptac- | AATTTTCACGGTCATATGATACCCTCGTGTGTGAGTTAATC | To clone the 149 bp ptac |
| F | (SEQ ID NO: 28) | promoter |
| catRBC-ptac- | GTGGGCATGGTGTGTCTATGGAGGTCAGGTATGATTACTAT | |
| R | TGA (SEQ ID NO: 29) | |
| catRBC-DF | GTGGATAACCGTATTACCGCAGGAAGTTGAGCAAGTCCGG | To clone the 1254 bp |
| | (SEQ ID NO: 30) | downstream DNA fragment |
| catRBC-DR | ACGAGGGTATCATATGACCGTGAAAATTTCCCACACTGC | of catRBCA operon from the |
| | (SEQ ID NO: 31) | P. putida EM42 chromosome |
| V-catRBC-F | GTGTCTTGCAGCACGCGCAG (SEQ ID NO: 32) | To verify the catRBC gene |
| | | deletion strain, wild strain |
| V-catRBC-R | CGCATGCACCGCAAGAACCTGTAC (SEQ ID NO: 33) | yields 2853 bp, the AcatRBC |
| | | yields 487 bp. |

For catRBCA gene deletion plasmid construction

| | | |
|---|---|---|
| catRBCA-UF | GACATAACCTCGAACACACACCATGCCCACAGGGG (SEQ | To clone the 1234 bp |
| | ID NO: 34) | upstream DNA fragment of |
| catRBCA-UR | GTAGCTGACATTCATCCGGCAAGGCGCAGGAAAAAGGGT | catRBCA gene from the P. |
| | TGC (SEQ ID NO: 35) | putida EM42 chromosome |
| catRBCA-DF | GTGGATAACCGTATTACCGCAATCGAGAAACTGCGAAAGC | To clone the 1270 bp |
| | TTGGC (SEQ ID NO: 36) | downstream DNA fragment |
| catRBCA-DR | GTGGGCATGGTGTGTGTTCGAGGTTATGTCACTGTGATTTT | of catRBCA operon from the |
| | GC (SEQ ID NO: 37) | P. putida EM42 chromosome |
| V-catRBCA-F | GGTAGAGATTGCGGATCAGCGACG (SEQ ID NO: 38) | To verify the catRBCA gene |
| V-catRBC-R | CGCATGCACCGCAAGAACCTGTAC (SEQ ID NO: 39) | deletion strain, wild strain |
| | | yields 3847 bp, the AcatRBCA |
| | | yields 426 bp. |

For phaZ gene deletion plasmid construction

| | | |
|---|---|---|
| phaZ-UF | TTTCCTGCGTTATCCCCTGATTCTGTGGATAGCAGACCTTCA | To clone the 1048 bp |
| | TCATCAGC (SEQ ID NO: 40) | upstream DNA fragment of |
| phaZ-UR | TGTCAGGCCGCAGCTGTTGCACGTGACTCTTGGGTG | phaZ gene from the P. putida |
| | (SEQ ID NO: 41) | EM42 chromosome |
| phaZ-DF | CACCCAAGAGTCACGTGCAACAGCTGCGGCCTGACA (SEQ | To clone the 1048 bp |
| | ID NO: 42) | downstream DNA fragment |
| phaZ-DR | CAGATAGCCCAGTAGCTGACATTCATCCGGCCGGCGCAAT | of phaZ gene from the P. |
| | TGCTTCTT (SEQ ID NO: 43) | putida EM42 chromosome |
| V-phaZ-F | TCTTTAACGGCATCGGC (SEQ ID NO: 44) | To verify the phaZ gene |
| V-phaZ-R | TGGCACCATTACCGCA (SEQ ID NO: 45) | deletion strain, wild strain |
| | | yields 308 bp, the AphaZ |
| | | yields 0 bp. |

For fadBAxE gene deletion plasmid construction

| | | |
|---|---|---|
| fadBAxE-UF | TTTCCTGCGTTATCCCCTGATTCTGTGGATAGCTGGGTATCA | To clone the 893 bp upstream |
| | CCAACCTG (SEQ ID NO: 46) | DNA fragment of fadBAEX1 |
| fadBAxE-UR | ATTTGCTTCCACTGGAGTACTTTCCTTTCAGACGCT (SEQ | operon from the P. putida |
| | ID NO: 47) | EM42 chromosome |
| fadBAxE-DF | CTGAAAGGAAAGTACTCCAGTGGAAGCAAATTCGCA (SEQ | To clone the 821 bp |
| | ID NO: 48) | downstream DNA fragment |
| fadBAxE-DR | CAGATAGCCCAGTAGCTGACATTCATCCGGCACATCGACTC | of fadBAEX1 operon from the |
| | GGCTATTCA (SEQ ID NO: 49) | P. putida EM42 chromosome |
| V-fadBAxE-F | GACCTCAAGAGCCTGACTGC (SEQ ID NO: 50) | To verify fadBAEX operon |
| V-fadBAxE-R | GCCGTGGATATTGACCTTGT (SEQ ID NO: 51) | deletion, wild strain yields |
| | | 966 bp, the A fadBAEX yields |
| | | 0 bp. |

For fadBA gene deletion plasmid construction

| | | |
|---|---|---|
| fadBA-UF | TTTCCTGCGTTATCCCCTGATTCTGTGGATATGTCCTTCATG | To clone the 1043 bp |
| | CGCGGC (SEQ ID NO: 52) | upstream DNA fragment of |
| fadBA-UR | TCCCGCTACGCGCAACTGATCTCCACGATATGGAAG (SEQ | fadBA1 operon from the P. |
| | ID NO: 53) | putida EM42 chromosome |
| fadBA-DF | TATCGTGGAGATCAGTTGCGCGTAGCGGGACAGCAG (SEQ | To clone the 1048 bp |
| | ID NO: 54) | downstream DNA fragment |
| fadBA-DR | CAGATAGCCCAGTAGCTGACATTCATCCGGGGCCCACAGC | of fadBA1 operon from the P. |
| | AGTGGCGA(SEQ ID NO: 55) | putida EM42 chromosome |
| V-fadBA-F | GTCTTCGATGCCACCGTG (SEQ ID NO: 56) | To verify fadBA1 operon |
| V-fadBA-R | GTTACCGGTCATGATCGCC (SEQ ID NO: 57) | deletion, wild strain yields |
| | | 712 bp, the AfadBA1 yields 0 |
| | | bp. |

For icd gene repression and Ptac-acn integration plasmid construction

| | | |
|---|---|---|
| icd-UF | CGTCAGATGGCTGCTTCTTCTAAGTAATTAGCGACCGGTTT | To clone the 1358 bp |
| | TCTGTTGCC (SEQ ID NO: 58) | upstream DNA fragment of |
| icd-UR | CAGATAGCCCAGTAGCTGACATTCATCCGGCGACAGCGGA | icd gene from the Putida |
| | GTAGTGCAAGCGTGGGATAC (SEQ ID NO: 59) | EM42 chromosome |
| icd-DF | TTTCCTGCGTTATCCCCTGATTCTGTGGATCACCTCATACCC | To clone the 931 bp upstream |
| | AAGTGGTC (SEQ ID NO: 60) | DNA fragment of icd gene |
| icd-DR | GGCACAGCGCAGGCAGTCAG (SEQ ID NO: 61) | from the Putida EM42 |
| | | chromosome |
| acn-Ptac-F | CTGCAAAAGCCTGACTGCCTGCGCTGTGCCGAGCTGTTGA | To clone the 157 bp ptac |
| | CAATTAATCATCGG (SEQ ID NO: 62) | promoter |
| acn-Ptac-R | GGAGTTCTTGCTTTCAGTCACAGTGAGCTCCATTTGATATA | |
| | CCCTTATATGTA (SEQ ID NO: 63) | |
| acn-F | ATGGAGCTCACTGTGACTGAAAGC (SEQ ID NO: 64) | To clone the 2835 bp acn |
| acn-R | TAATTACTTAGAAGAAGCAGCCAT (SEQ ID NO: 65) | gene from the C. glutamicum |
| | | ATCC 13032 chromosome |

For pBb(B5)1k-tpa plasmid construction

| | | |
|---|---|---|
| Ptac-TPA-F | GGCAATTCCGACGTCCTATGGAGGTCAGGTATGATTACTAT | To clone the 143 bp ptac |
| | TGA (SEQ ID NO: 66) | promoter |
| ptac-TPA-R | GCTTTCCATATGATACCCTCGTGT (SEQ ID NO: 67) | |
| TPA-F | ACACGAGGGTATCATATGGAAAGCAGCGTTGTCGACACGG | To clone the 5218 bp tpa |
| | T (SEQ ID NO: 68) | operon from Rhodococcus |
| TPA-R | CGAGTTTGGATCCCTACTTGCGGGCGAGCGAATGACTTT | jostii RHA1 |
| | (SEQ ID NO: 69) | |
| BR5-F | TCGCCCGCAAGTAGGGATCCAAACTCGAGTAAGGAT (SEQ | To clone the 2517 bp BR5 |
| | ID NO: 70) | origin from pBb(B5)1k-GFPuv |
| BR5-R | CTGACCTCCATAGGACGTCGGAATTGCCAGC (SEQ ID | plasmid |
| | NO: 71) | |

For pSEVA421-aroY-ectB plasmid construction

| | | |
|---|---|---|
| pSEVA-F | ACTAGTCTTGGACTCCTGTTGATAG (SEQ ID NO: 72) | To clone the 3865 bp RK2 |
| Ptac-R | ATGATACCCTCGTGTGTGAGTTAAT (SEQ ID NO: 73) | origin from pSEVA421 |
| | | plasmid |
| aroY-frag1 | ATTAACTCACACACGAGGGTATCATATGCAGAACCCGATCA | Synthesized DNA fragment 1 |
| | ACGACCTGCGCTCCGCGATCGCGCTGCTGCAACGCCATCC | of aroY. Gibson overlaps are |
| | GGGTCACTACATCGAAACCGACCACCCGGTCGACCCGAAC | underlined. |
| | GCCGAACTGGCCGGTGTGTACCGCCACATCGGTGCGGGT | |
| | GGCACCGTGAAACGTCCGACCCGCACCGGTCCAGCCATG | |
| | ATGTTCAACAGCGTGAAGGGCTACCCAGGCAGCCGCATCC | |
| | TGGTGGGCATGCACGCCAGCCGTGAACGTGCCGCCCTGCT | |
| | GCTGGGCTGCGTGCCAAGCAAACTGGCGCAGCACGTGGG | |
| | CCAGGCCGTGAAGAACCCGGTGGCCCCAGTGGTGGTGCC | |
| | AGCCAGCCAAGCCCCATGCCAAGAACAGGTGTTCTACGCC | |
| | GACGACCCGGACTTCGACCTGCGCAAGCTGCTGCCAGCC | |
| | CCAACCAACACCCCGATCGATGCCGGTCCGTTCTTCTGCCT | |
| | GGGCCTGGTGCTGGCGAGCGACCCGGAAGATACCAGCCT | |
| | GACCGACGTGACCATCCACCGCCTGTGCGTGCAAGAGCGC | |
| | GACGAGCTGAGCATGTTCCTGGCCGCCGGTCGCCACATCG | |
| | AGGTGTTCCGCAAGAAGGCCGAAGCCGCCGGTAAGCCGC | |
| | TGCCGGTGACCATCAACATGGGCCTGGACCCAGCCATCTA | |
| | CATCGGTGCCTGCTTCGAAGCGCCAACCACCCCGTTCGGC | |
| | TACAACGAGCTGGGTGTGGCC (SEQ ID NO: 74) | |
| aroY-frag2 | ACAACGAGCTGGGTGTGGCCGGTGCCCTGCGTCAGCAAC | Synthesized DNA fragment 2 |
| | CGGTGGAACTGGTGCAGGGCGTGGCCGTGAAAGAGAAG | of aroY. Gibson overlaps are |
| | GCGATCGCGCGTGCCGAGATCATCATCGAGGGCGAACTGC | underlined. |
| | TGCCAGGCGTGCGCGTGCGCGAAGATCAGCACACCAACA | |
| | CCGGTCACGCCATGCCGGAATTCCCAGGCTACTGCGGTGA | |
| | GGCCAACCCGAGCCTGCCGGTGATCAAGGTGAAGGCCGT | |
| | GACCATGCGCAACCACGCCATCCTGCAGACCCTGGTGGGT | |
| | CCGGGTGAGGAACACACCACCCTGGCGGGTCTGCCGACC | |
| | GAAGCCAGCATCCGCAACGCCGTGGAAGAGGCGATCCCA | |
| | GGCTTCCTGCAGAACGTGTACGCCCACACCGCCGGTGGC | |
| | GGTAAGTTCCTGGGCATCCTGCAGGTCAAGAAGCGCCAG | |
| | CCGAGCGACGAAGGCCGTCAGGGCCAAGCCGCCCTGATC | |
| | GCCCTGGCCACCTACAGCGAGCTGAAGAACATCATCCTGG | |
| | TGGACGAGGACGTGGACATCTTCGACAGCGACGACATCCT | |
| | GTGGGCCATGACCACCCGCATGCAGGGCGACGTGAGCAT | |
| | CACCACCCTGCCAGGCATCCGTGGCCATCAGCTGGACCCG | |
| | AGCCAGAGCCCAGACTACAGCACCAGCATCCGTGGCAAC | |
| | GGCATCAGCTGCAAGACCATCTTCGACTGCACCGTGCCGT | |
| | GGGCCCTGAAAGCCCGTTTCGAGCGTGCCCCATTCATGGA | |
| | AGTGGACCCGACCCCGTGGGCCCCAGAGCTGTTCAGCGA | |
| | CAAGAAGTGAGGAGGTTCGAATATGCGCCT (SEQ ID | |
| | NO: 75) | |
| ecdB-frag | CAAGAAGTGAGGAGGTTCGAATATGCGCCTGATCGTGGG | Synthesized DNA fragment of |
| | CATGACCGGTGCGACCGGTGCGCCACTGGGCGTGGCCCT | ecdB. Gibson overlaps are |
| | GCTGCAGGCCCTGCGTGATATGCCGGAAGTGGAAACCCAC | underlined. |
| | CTGGTGATGAGCAAGTGGGCCAAGACCACCATCGAGCTG | |
| | GAAACCCCGTACACCGCCCAGGACGTGGCCGCCCTGGCC | |
| | GATGTGGTGCATAGCCCAGCCGATCAAGCCGCCACCATCA | |
| | GCAGCGGCAGCTTCCGCACCGACGGCATGATCGTGATCCC | |
| | GTGCAGCATGAAAACCCTGGCCGGTATCCGTGCCGGTTAC | |
| | GCCGAAGGCCTGGTGGGTCGTGCCGCCGACGTGGTGCTG | |
| | AAAGAGGGTCGCAAGCTGGTCCTGGTGCCACGCGAAACC | |
| | CCACTGAGCACCATCCACCTGGAAAACATGCTGGCCCTGA | |
| | GCCGCATGGGCGTGGCCATGGTCCCACCGATGCCAGCCTA | |
| | CTACAACCACCCGCAGACCGCCGACGACATCACCCAGCAC | |
| | ATCGTGACCCGTGTGCTGGACCAGTTCGGCCTGGAACACA | |
| | AGAAAGCCCGTCGCTGGAACGGCCTGCAAGCCGCCAAGC | |
| | ACTTCAGCCAAGAGAACAACGACGGCATCTGAACTAGTCT | |
| | TGGACTCCTGTTGATAG (SEQ ID NO: 76) | |

For pSEVA421-phaG-alkK-phaC1-phaC2 plasmid construction

| | | |
|---|---|---|
| EM7-F | CCTACGTGCTGACCCGATGAGCGTCGTGACTGGGAAAACC | To clone the 3857 bp RK2 |
| | (SEQ ID NO: 77) | origin from pSEVA421 |
| EM7-R | ATTTCTGGCCTCATGGTATATTCCTCTTGCTAGAATTCGC | plasmid |
| | (SEQ ID NO: 78) | |
| phaG-F | AGCAAGAGGAATATACCATGAGGCCAGAAATCGCTGTACT | To clone the 924 bp phaG |
| | (SEQ ID NO: 79) | gene from the P. putida EM42 |
| phaG-R | TCCCTTTTCTGAAAAGCGGTCAGATGGCAAATGCATGCTG | chromosome |
| | (SEQ ID NO: 80) | |
| alk-F | GCATGCATTTGCCATCTGACCGCTTTTCAGAAAAGGGATC | To clone the 1745 bp alk gene |
| | (SEQ ID NO: 81) | from the P. putida EM42 |
| alk-R | GACGCTCCGTTGTCCTGAGTTACAACGTGGAAAGGAACGC | chromosome |
| | (SEQ ID NO: 82) | |
| phaC1-F | TTCCTTTCCACGTTGTAACTCAGGACAACGGAGCGTCGTA | To clone the 1739 bp phaC1 |
| | (SEQ ID NO: 83) | gene from the P. putida EM42 |
| phaC1-R | ACTCCCTCGTCTGATCCATCAACGCTCGTGAACGTAGGTG | chromosome |
| | (SEQ ID NO: 84) | |
| phaC2-F | TACGTTCACGAGCGTTGATGGATCAGACGAGGGAGTGTTG | To clone the 1744 bp phaC2 |
| | (SEQ ID NO: 85) | gene from the P. putida EM42 |
| phaC2-R | GTTTTCCCAGTCACGACGCTCATCGGGTCAGCACGTAGGT | chromosome |
| | (SEQ ID NO: 86) | |

For pSEVA421-cadA-phaG-alkK-phaC1-phaC2 plasmid construction

| | | |
|---|---|---|
| cadA-F1 | GCGCCAGGCACCTACGTGCTGACCCGATGAGTCCTTTTCC | To clone the 1602 bp |
| | GCTGCATAACCCT (SEQ ID NO: 87) | fragment from pSEVA421- |
| cadA-R1 | CCCTGCAAAGTAAACTGGATGGC (SEQ ID NO: 88) | cadA plasmid |
| cadA-F2 | CATCAGATCCTTGGCGGCAAGA (SEQ ID NO: 89) | To clone the 3856 bp |
| cadA-R2 | TATACTATGCCGATGATTAATTGTCAACAATTGTCTTATTTGC | fragment from pSEVA421- |
| | CGACTACCTT (SEQ ID NO: 90) | cadA plasmid |
| PHA-F | TTGTTGACAATTAATCATCGGCATAG (SEQ ID NO: 91) | To clone the 6115 bp |
| PHA-R | TCATCGGGTCAGCACGTAGGT (SEQ ID NO: 92) | fragment from pSEVA421- |
| | | cadA-phaG-alkK-phaC1- |
| | | phaC2 plasmid |

Routine cultures of Pseudomonas putida and Escherichia coli for plasmid construction and preculture were grown in 5 mL Luria Broth (LB) medium with appropriate antibiotics (50 μg/mL kanamycin or 100 μg/mL streptomycin) and at 30 and 37 °C, respectively. All batch fermentations were conducted using 50 mL modified M9 minimal medium supplemented with specific carbon sources at 30 °C and 250 rpm.

Plasmid and strain construction. All synthetic oligonucleotides and DNA fragments were ordered from Integrated DNA Technologies (IDT, Coralville, IA). Polymerase chain reaction (PCR) was performed using Q5@ High-Fidelity DNA Polymerase (NEB, Ipswich, MA). DNA sequencing services were provided by GENEWIZ from Azenta Life Sciences (Chelmsford, MA). Plasmids were assembled via Gibson Assembly and directly transformed into relative P. putida strains. For gene integration, plasmids were constructed using the vector pK18mobsacB and transformed into E. coli DH10B competent cells for plasmid amplification. All of the plasmids constructed in this study were verified using restriction digestion and PCR sequencing.

To construct the Pp-T strain, the ped operon in P. putida EM42 was first knocked out using the plasmid pK18-ped to derive the strain Pp01. Here, pK18-ped was created by cloning the upstream and downstream homology regions of the ped operon from P. putida EM42 with the primers ped-UF/UR and ped-DF/DR into the vector pK18mobsacB. Pp01 was then confirmed by colony PCR with the primers V-ped-F/R. Meanwhile, after amplification with the primers P_{tac}-TPA-F/R and TPA-F/R, the tac promoter (P_{tac}) and tpa operon, including tpaAa, tpaAb, tpaC, tpaB, and tpaK, from Rhodococcus jostii RHA1 were ligated into the pBb (B5) 1k-GFPuv backbone using Gibson assembly to generate the plasmid pBb (B5) 1k-tpa. Finally, pBb (B5) 1k-tpa was transformed into P. putida EM42 and Pp01 to yield the strains Pp-T₀ and Pp-T for TPA degradation.

The Pp-E strain was created by first deleting gclR, the repressor gene of the Gcl pathway, from the P. putida M31 chromosome using the plasmid pK18-gclR. Here, pk18-glcR was constructed by amplifying the upstream and downstream regions of the gclR gene by PCR with the primers gclR-UF/UR and gclR-DF/DR, and inserting them into the vector pK18mobsacB. After counter-selection with sucrose, the loss of the gclR gene was confirmed with colony PCR using the primes V-gclR-F/R. The resulting strain was named PpM01. Similarly, to replace the native promoter of glcDEF with the tac promoter, the upstream and downstream homologous regions of the native promoter were amplified by PCR with the primers glcDEF-UF/UR and glcDEF-DF/DR and inserted into the vector pK18mobsacB to derive the plasmid pK18-Pro-glcDEF. Subsequently, the native glcDEF promoter of PpM01 was replaced by the tac promoter using pK18-Pro-glcDEF, resulting in the strain Pp-E for EG degradation.

The Pp-TE strain for TPA and EG co-degradation was obtained by transforming the plasmid pBb (B5) 1k-tpa into Pp-E.

Strain construction for mcl-PHA production: To make the plasmid pSEVA421-phaG-alkK-phaC1-phaC2, the pSEVA421 backbone with the promoter P_{EM7} was first amplified with EM7-F/R. Then, the genes phaG, alkK, phaC1, and phaC2 were amplified from the genomic DNA of P. putida EM42 using the following primers: phaG-F/R, alkK-F/R, phaC1-F/R, and phaC2-F/R, respectively. These five PCR products were circularized by Gibson assembly to obtain the plasmid pSEVA421-phaG-alkK-phaC1-phaC2, which was transformed via electroporation into Pp-T, Pp-E, and Pp-TE to derive the strains Pp-TP, Pp-EP, and Pp-TEP, respectively. On the other hand, genome editing was performed as follows. The upstream and downstream homologous regions (800-1,100 bp) of fadBA, fadBAxE, and phaZ were amplified using the primers fadBA-UF/UR, fadBA-DF/DR, fadBAxE-UF/UR, fadBAxE-DF/DR, phaZ-UF/UR and phaZ-DF/DR, respectively. Each pair of the corresponding homologous regions was combined with the pK18mobSacB backbone to create the plasmids pK18-fadBA, pK18-fadBAxE, and pK18-phaZ. These plasmids were subsequently used for the deletion of fadBA, fadBAxE, and phaZ in Pp01 and Pp-E. The resulting strains, Pp05 and PpΔ-E, were confirmed by colony PCR with the primers V-fadBA-F/R, V-fadBAxE-F/R, and V-phaZ-F/R. Later, pSEVA421-phaG-alkK-phaC1-phaC2 was transformed into PpΔ-E, yielding the strain PpΔ-EP. Meanwhile, pBb (B5) 1k-tpa and pSEVA421-phaG-alkK-phaC1-phaC2 were both transformed into Pp05 and PpΔ-E to generate the strains PpΔ-TP and PpΔ-TEP. To construct control strains, the plasmid pSEVA421 was transformed into Pp-T, Pp-E, and Pp-TE, resulting in the strains Pp-TS, Pp-ES, and Pp-TES, respectively.

Strain construction for MA production: The plasmid pK18-catRBCA was constructed by cloning the upstream and downstream homology regions of the catRBCA cluster from P. putida EM42 with the primers catRBCA-UF/UR and catRBCA-DF/DR into the vector pK18mobsacB. The plasmid was then used to knock out the entire catRBCA cluster in Pp01 to eliminate MA utilization. The resulting strain, Pp07, was confirmed by colony PCR with the primers V-catRBCA-F/R. To construct the plasmid pSEVA421-aroY-ecdB, codon-optimized aroY and ecdB from Enterobacter cloacae ATCC 13047 were synthesized by IDT and assembled into pSEVA421 that contains the tac promoter. Finally, pSEVA421-aroY-ecdB and pBb (B5) 1k-tpa were both transformed into Pp07 to yield the strain Pp-TC. To integrate the tac promoter upstream of catA and delete catRBC, the 5′ and 3′ homology regions of catRBC were amplified from PpM01 with the primers catRBC-UF/UR and catRBC-DF/DR. The tac promoter was amplified from pBb (B5) 1k-tpa using the primers catRBC-ptac-F/R. Then, these fragments were assembled into the vector pK18mobsacB, resulting the plasmid pK18-catRBC. Afterwards, pK18-catRBC was transformed into PpM02 to yield PpM09. Finally, pSEVA421 and pBb (B5) 1k-tpa were further transformed along with pSEVA421-aroY-ecdB into PpM09, generating the strains Pp-EM and Pp-TEM, respectively.

Strain construction for MA and IA co-production: To alter the start codon of icd and integrate heterologous acn, the upstream and downstream homologous regions (900-1,200 bp) of icd and acn gene were amplified from the genomic DNA of P. putida EM42 and C. glutamicum ATCC 13032, respectively, using the primers icd-UF/UR, icd-DF/DR, and acn-F/R. And the tac promoter and RBS was amplified from pBb (B5) 1k-tpa using the primers of acn-ptac-F/R. These four fragments were then ligated into pK18mobsacB and created the plasmid pK18-icdGTG-acn. The yielded plasmid was subsequently transformed into Pp01 and Pp-E to derive the strains Pp-01A and Pp-EA, respectively. Later, pBb (B5) 1k-tpa was transformed into Pp01A to yield the strain Pp-TA. To make the plasmid pSEVA421-cadA-phaG-alkK-phaC1-phaC2, the backbone with P_{EM7}-cadA and P_{EM7}-phaG-alkK-phaC1-phaC2 were amplified from pSEVA421-cadA and pSEVA421-phaG-alkK-phaC1-phaC2, respectively, with cadA-F1/R1, cadA-F2/R2, and PHA-F/R. The circularized plasmid was transformed into Pp-TA and Pp-EA, yielding the strains Pp-TAPI and Pp-EAPI, respectively. To construct control strains for mcl-PHA production only, the plasmid pSEVA421-phaG-alkK-phaC1-phaC2 was transformed into Pp-TA and Pp-EA, resulting in the strains Pp-TAP and Pp-EAP, respectively.

Transformation of constructs into P. putida strains. Electroporation was used for plasmid transformation. After overnight cultivation in 5 mL LB medium at 30 °C and 250 rpm, 1 mL P. putida cells were centrifuged and washed twice with 0.2 mL of 0.3 M sucrose solution. Then, cells pellets were resuspended in 2 mL of 0.3 M sucrose solution. Plasmid DNAs (0.2-2 μg) were mixed with 100 μL cell suspension in 0.1 cm electroporation cuvette (Bio-Rad) and electroporated at 1.6 kV by Eporator (Eppendorf, Germany). Subsequently, cells were transferred to 1 mL LB medium for recovery at 30 °C and 250 rpm for 1-2 h. 100 μL recovered cells were plated on LB agar plates containing appropriate antibiotics and incubated at 30 °C overnight. Colonies were picked up from agar plates to selective LB medium and grown at 30 °C and 250 rpm overnight for further verification by target fragment PCR and plasmid digestion. For sucrose counter-selection, colonies selected on LB plates were incubated at 30 °C and 250 rpm for 4-6 h. Then, 100 μL cells were plated on LB+2% sucrose agar plates overnight. Finally, 20-40 colonies were selected to screen for correct gene replacements or knock-out by PCR.

Characterization of TPA and EG degradation. Engineered P. putida strains from glycerol stocks were grown overnight in 5 mL LB medium containing appropriate antibiotics at 30 °C. 2% (v/v) of cells were further inoculated into 50 mL LB medium as second seed culture and cultivated at 30 °C and 250 rpm for 10-12 h. For monoculture cultivation, precultures were then washed twice with distilled water and inoculated at OD₆₀₀ of 0.2 in 50 mL modified M9 minimal medium with TPA and/or EG as the carbon sources at 30 °C and 250 rpm. For co-culture cultivation, cell centrifugation, washing, and resuspension were performed as for monoculture. The initial OD₆₀₀ of the co-culture after inoculation was controlled at 0.2 and the Pp-T:Pp-E ratio was fixed at 1:1 or 3:1, unless otherwise noticed. Fermentation samples (1 mL) were collected at 12 h (or shorter when TPA/EG were consumed mostly) intervals to determine cell growth and TPA and EG concentrations.

Depolymerization of PET via alkaline hydrolysis. PET powder (ES30-PD-000131) purchased from Goodfellow was used for depolymerization. Briefly, PET powder with 1-20% aqueous NaOH in 500 mL glass bottle was autoclaved for depolymerization in an autoclave reactor at 131 °C for 2 h. After cooling down, the solution was analyzed in terms of its TPA and EG concentrations via high-performance liquid chromatography system (HPLC). The highest depolymerization reaction was reached at a 5% NaOH solution mixed with benzalkonium chloride (BKC) as phase transfer catalyst, which was chosen for the following experiments. Batch fermentations with depolymerized PET as the substrate were studied in 250 ml shaker flasks, with each containing 50 ml of modified M9 medium. The depolymerized PET solution with M9 medium was neutralized with HCl and the addition volume of the solution was based on the required TPA and EG concentrations.

mcl-PHA characterization and quantification. For mcl-PHA production, precultures were prepared as described above and inoculated at an OD₆₀₀ of 0.2-0.4 in modified M9 minimal medium. Nitrogen-limiting condition was prepared by substituting NH₄Cl from 1 g/L to 0.6 g/L in M9 medium. For fed-batch fermentation, pulse addition of feed media (PET hydrolysate and M9 salts) was conducted when TPA/EG were consumed mostly. 40 mL of fermentation broth were taken when TPA and/or EG were totally consumed, and centrifuged at 10,000 g for 10 min. The cell pellets were then washed with distilled water and lyophilized for PHA extraction. For mcl-PHA composition analysis, 30 mg of lyophilized cells were suspended in 2 mL of sulfuric acid-methanol solution (15%, v/v) and 2 mL of chloroform. The mixture was sealed and placed in a heating block at 100 °C for 4 h. After cooling to room temperature, 1 mL purified water was added to form a two-phase liquid. Then, the organic phase liquid containing the resulting methyl esters was transferred into GC vials and analyzed by gas chromatography mass spectroscopy (GC-MS) using a SHIMADZU GC2010 equipped with GCMS-QP2010 Plus (Tokyo, Japan). Here, the separation was carried out with a 30-m long column (J&W DB-5 ms, film thickness: 0.25 μm; diameter: 0.25 mm) from Agilent (Santa Clara, CA). The GC-MS method was used as previously described with minor modifications⁴⁷. Helium was used as the carrier gas with a flow rate of 1 mL/min. The injection, ion source, and interface temperature were maintained at 250 °C and the mass spectra were recorded under the scan mode in the range of 35-550 m/z. The column oven was held at 35 °C for 5 min, heated to 225 °C at 15 °C/min, held at 225 °C for 2 min, then ramped again to 250 °C at 15 °C/min and finally held at 250 °C for 5 min. All chemicals for mcl-PHA standards, including Methyl 3-hydroxyhexanoate (6C), 3-hydroxyoctanoic acid (8C), 3-hydroxydecanoic acid (10C), 3-hydroxydodecanoate (12C), and 3-hydroxytetradecanoate (14C), were purchased from Sigma Aldrich.

Characterization of labor partition for MA production. Starter cultures were prepared as described above and inoculated in 50 mL modified M9 medium, supplemented with either 56 mM TPA/EG or PET hydrolysate (final TPA/EG is around 50 mM), at 30 °C and 250 rpm. To vary the initial population ratio, the Pp-TC and Pp-EM monocultures were washed twice and their cell densities were measured. Then, the two cultures were mixed at different ratios (3:1, 1:1, 1:3, 1:5, and 1:9) with a starting total OD₆₀₀ of 0.2. To vary the inoculation lag time, the Pp-EM monoculture was first washed twice with distilled water and inoculated at OD₆₀₀ of 0.1 in 50 mL selected M9 minimal medium at 30 °C and 250 rpm. After 0, 12, 24, or 36 h, precultured Pp-TC was collected, washed, and inoculated at OD₆₀₀ of 0.1 into the Pp-EM culture. Fermentation samples (1 mL) were collected at 12 h intervals to determine TPA, EG, CAT, and MA concentrations.

Aromatics and MA quantification. TPA, CAT, and cis-cis MA samples were collected after culture centrifugation at 15,000 g for 10 min. Then, supernatants were diluted 50 times before being injected in the HPLC (Shimadzu, Japan) equipped with a UV detector. The related metabolites were separated on a C18 LC column (Phenomenex Luna 5 μm C18 (2), 100 Å, 150×4.6 mm, Torrance, CA) at 40 °C and a flow rate of 0.5 mL/min. The following gradients of buffer A (0.005% Formic acid) and buffer B (20% Acetonitrile) were used: buffer B was first increased from 60 to 80% for 2 min, then ramped to 100% for 6 min, and held at 100% for 12 min. TPA, CAT, and MA were detected at 260 nm with the quantifications being realized using external standards. The standard curves were generated by measuring 0.001-0.1 g/L disodium terephthalate (99.0+%, TCI America™), 0.05-0.5 g/L CAT (≥99.0%, Sigma Aldrich), and 0.001-0.1 g/L MA ((≥97.0%, Sigma Aldrich), respectively.

EG quantification. Samples were first centrifuged at 15,000 g for 10 min, and then the supernatants were diluted 10 times and determined by HPLC (Agilent Technologies 1260 Infinity system, Santa Clara, CA) equipped with a refractive index detector (RID) and a Rezex™ ROA-Organic Acid H+ (8%) LC column (Phenomenex Inc., Torrance, CA). The column was eluted with 5 mM of sulfuric acid as a mobile phase at a flow rate of 0.6 mL/min and 50 °C. The pure EG purchased from Thermo Fisher Scientific (Waltham, MA) was used to establish the calibration curve for EG concentration calculation.

IA quantification. Samples were first centrifuged at 15,000 g for 10 min, and then the supernatants were diluted 10 times and determined by HPLC (Shimadzu, Japan) equipped with a Aminex HPX 87H column (Bio-Rad) and a UV detector at 210 nm. The column was eluted with 2.5 mM of sulfuric acid as a mobile phase at a flow rate of 0.5 ml/min and 85 °C. The pure IA purchased from Thermo Fisher Scientific (Waltham, MA) was used to establish the calibration curve for IA concentration calculation.

### Statistical Analysis

All experiments were performed multiple times as mentioned in figure legends. The results are presented as mean values with standard deviations, unless stated otherwise. All statistical analysis was performed using GraphPad Prism 9.

Amino Acid Abbreviations

| | | |
|---|---|---|
| alanine | Ala | A |
| arginine | Arg | R |
| asparagine | Asn | N |
| aspartic acid | Asp | D |
| cysteine | Cys | C |
| glutamic acid | Glu | E |
| glutamine | Gln | Q |
| glycine | Gly | G |
| histidine | His | H |
| isoleucine | lle | \| |
| leucine | Leu | L |
| lysine | Lys | K |
| methionine | Met | M |
| phenylalanine | Phe | F |
| proline | Pro | P |
| serine | Ser | S |
| threonine | Thr | T |
| tryptophan | Trp | W |
| tyrosine | Tyr | Y |
| valine | Val | V |

We claim:

1. A recombinant Pseudomonas putida group bacterium comprising a deleted or non-functional ped operon and recombinant nucleic acid molecules encoding a large subunit of terephthalate 1,2-dioxygenase (tpaAa), small subunit of terephthalate 1,2-dioxygenase (tpaAb), ferredoxin reductase subunit (tpaB), dihydrodiol dehydrogenase (tpaC), and terephthalate transporter (tpaK).

2. The recombinant Pseudomonas putida group bacterium of claim 1, wherein the nucleic acid molecules encoding tpaAa, tpaAb, tpaB, tpaC, and tpaK are from Rhodococcus jostii.

3. A recombinant Pseudomonas putida group bacterium comprising a deleted or non-functional repressor gene of the glycolate dehydrogenase (Gcl) pathway (gclR) and a recombinant strong constitutive promoter operably linked to a glcDEF operon.

4. The recombinant Pseudomonas putida group bacterium of claim 3, wherein the strong constitutive promoter is Tac Promoter (P_{tac}).

5. The recombinant Pseudomonas putida group bacterium of claim 3, further comprising a recombinant ribosome binding site operably linked to the glcDEF operon.

6. A bacterial culture comprising the Pseudomonas putida group bacterium of claim 1 and the Pseudomonas putida group bacterium of claim 3.

7. A method of degrading polyethylene terephthalate (PET) comprising mixing hydrolyzed PET product with a growth medium, and adding the bacterial culture of claim 6 to a mixture of the hydrolyzed PET product and the growth medium.

8. The method of claim 7 wherein the hydrolyzed PET product is formed by depolymerizing PET by hydrolysis.

9. The method of claim 8, wherein the hydrolysis comprises contacting the PET with 0.5 to 5% NaOH.

10. The method of claim 7, wherein 50% or more of terephthalic acid (TPA) and ethylene glycol (EG) present in the hydrolyzed PET product is degraded.

11. A recombinant Pseudomonas putida group bacterium comprising:
(i) a deleted or non-functional ped operon;
(ii) recombinant nucleic acid molecules encoding tpaAa, tpaAb, tpaB, tpaC, and tpaK; and
(iii) recombinant nucleic acid molecules encoding hydroxyacyl-ACP acyltransferase (phaG), acyl-CoA-synthase (alkK), mcl-PHA synthase 1 (phaC1) and mcl-PHA synthase 2 (phaC2).

12. A recombinant Pseudomonas putida group bacterium comprising:
(i) a deleted or non-functional gclR gene;
(ii) a recombinant strong constitutive promoter operably linked to a glcDEF operon; and
(iii) recombinant nucleic acid molecules encoding phaG, alkK, phaC1 and phaC2.

13. A bacterial culture comprising the Pseudomonas putida group bacterium of claim 11 and the Pseudomonas putida group bacterium of claim 12.

14. A method of making medium chain length polyhydroxyalkanoates (mcl-PHA (C6 to C14)) comprising mixing a hydrolyzed PET product with a growth medium, and adding the bacterial culture of claim 13 to a mixture of the hydrolyzed PET product and the growth medium, wherein mcl-PHA (C6-C14) is produced.

15. The method of claim 14, wherein the hydrolyzed PET product is formed by depolymerizing PET by hydrolysis.

16. The method of claim 15, wherein the hydrolysis comprises contacting the PET with 0.5 to 5% NaOH.

17. A recombinant Pseudomonas putida group bacterium comprising:
(i) a deleted or non-functional ped operon;
(ii) recombinant nucleic acid molecules encoding tpaAa, tpaAb, tpaB, tpaC, and tpaK.
(iii) a deleted or non-functional catRBCA cluster;
(iv) a recombinant nucleic acid molecule encoding PCA decarboxylase (aro Y); and
(v) a recombinant nucleic acid molecule encoding decarboxylase enhancer (ecdB).

18. A recombinant Pseudomonas putida group bacterium comprising:
(i) a deleted or non-functional gclR gene;
(ii) a recombinant strong constitutive promoter operably linked to a glcDEF operon;
(iii) a deleted or non-functional catRBCA cluster; and
(iv) a recombinant strong constitutive promoter operably linked to a nucleic acid molecule encoding major catechol 1,2-dioxygenase (catA).

19. A bacterial culture comprising the Pseudomonas putida group bacterium of claim 17 and the Pseudomonas putida group bacterium of claim 18.

20. A method of making muconate (MA) comprising mixing a hydrolyzed PET product with a growth medium, and adding the recombinant Pseudomonas putida group bacterium of claim 17 and the recombinant Pseudomonas putida group bacterium of claim 18 to a mixture of the hydrolyzed PET product and the growth medium, wherein MA is produced.

21. The method of claim 20, wherein the hydrolyzed PET product is formed by depolymerizing PET by hydrolysis.

22. The method of claim 21, wherein the hydrolysis comprises contacting the PET with 0.5 to 5% NaOH.

23. The method of claim 20 wherein the recombinant Pseudomonas putida group bacterium of claim 18 is added to the mixture of the hydrolyzed product and the growth medium before the recombinant Pseudomonas putida group bacterium of claim 17 is added to the mixture.

24. A recombinant P. putida group bacterium comprising a deleted or non-functional ped operon and recombinant nucleic acid molecules encoding a large subunit of terephthalate 1,2-dioxygenase (tpaAa), small subunit of terephthalate 1,2-dioxygenase (tpaAb), ferredoxin reductase subunit (tpaB), dihydrodiol dehydrogenase (tpaC), and terephthalate transporter (tpaK), —reduced expression of icd, a recombinant nucleic acid molecule encoding acn, recombinant nucleic acid molecules encoding phaG, alkK, phaC1, and phaC2, which are operably linked to one or more strong constitutive promoters and a recombinant nucleic acid molecule encoding cadA.

25. The recombinant P. putida group bacterium of claim 24, wherein the one or more strong constitutive promoters are P_{tac}.

26. The recombinant P. putida group bacterium of claim 24, wherein the reduced expression of icd is due to a weak start codon or a recombinant weak promoter.

27. A recombinant P. putida group bacterium comprising a deleted or non-functional repressor gene of the glycolate dehydrogenase (Glc) pathway (glcR), and a recombinant strong constitutive promoter operably linked to a glcDEF operon, a recombinant ribosome binding site operably linked to the glcDEF operon, reduced expression of icd, a recombinant nucleic acid molecule encoding acn, recombinant nucleic acid molecules encoding phaG, alkK, phaC1, and phaC2 operably linked to one or more strong or constitutive promoters, and a recombinant nucleic acid molecule encoding cadA.

28. The recombinant P. putida group bacterium of claim 27, wherein the reduced expression of icd is due to a weak start codon or a recombinant weak promoter.

29. The recombinant P. putida group bacterium of claim 27, wherein the strong constitutive promoter is P_{tac}

30. A bacterial culture comprising the Pseudomonas putida group bacterium of claim 24 and the Pseudomonas putida group bacterium of claim 27.

31. A method of simultaneous mcl-PHA and itaconic acid production comprising mixing a hydrolyzed PET product with a growth medium, and adding the recombinant Pseudomonas putida group bacterium of claim 24 and the recombinant Pseudomonas putida group bacterium of claim 27 to a mixture of the hydrolyzed PET product and the growth medium, wherein mcl-PHA and itaconic acid is produced.

32. The method of claim 31, wherein the hydrolyzed PET product is formed by depolymerizing PET by hydrolysis.

33. The method of claim 32, wherein the hydrolysis comprises contacting the PET with 0.5 to 5% NaOH.
